(19) United States
(12) Patent Application Publication
(10) Pub. No.: US 2024/0197858 A1
(43) Pub. Date: Jun. 20, 2024

(54) NIPAH VIRUS IMMUNOGENS AND THEIR USE

(71) Applicant: The U.S.A as represented by the Secretary, Department of Health and Human Service, Bethesda, MD (US)

(72) Inventors: Barney Graham, Smyrna, GA (US); Rebecca Loomis, Philadelphia, PA (US); Guillaume Stewart-Jones, Cambridge, MA (US); John Mascola, Rockville, MD (US)

(73) Assignee: The U.S.A as represented by the Secretary, Department of Health and Human Service, Bethesda, MD (US)

(21) Appl. No.: 18/424,606

(22) Filed: Jan. 26, 2024

Related U.S. Application Data
(62) Division of application No. 17/261,828, filed on Jan. 20, 2021, now Pat. No. 11,890,339.
(60) Provisional application No. 62/714,230, filed on Aug. 3, 2018.

(51) Int. Cl. A61K 39/155; A61P 31/14; C07K 14/005

(52) U.S. Cl. CPC A61K 39/155; A61P 31/14; C07K 14/005; A61K 2039/70

(57) ABSTRACT

Embodiments of immunogens comprising a multimer of NiV G ectodomains and protein nanoparticles comprising an NiV G ectodomain are provided. Also disclosed are nucleic acids encoding the immunogens and methods of their production. Methods for inducing an immune response in a subject by administering a disclosed immunogen to the subject are also provided. In some embodiments, the immune response treats or inhibits NiV infection in a subject.

### CROSS REFERENCE TO RELATED APPLICATIONS

This is a divisional of U.S. patent application Ser. No. 17/261,828, filed on Jan. 20, 2021, which is the U.S. National Stage of International Application No. PCT/US2019/045110, filed Aug. 5, 2019, which was published in English under PCT Article 21(2), which in turn claims priority to U.S. Provisional Application No. 62/714,230, filed Aug. 3, 2018. The entire contents of each of the above-identified applications is incorporated herein by reference in its entirety.

### SEQUENCE LISTING

The Sequence Listing is submitted as XML file in the form of the file named “4239-100112-08_Sequence.xml” (150,343 bytes), which was created on Jan. 26, 2024, which is incorporated by reference herein.

### FIELD

This disclosure relates to polypeptides, polynucleotides, compositions, and methods of their use, for elicitation and detection of an immune response to Nipah virus (NiV).

### BACKGROUND

NiV is an enveloped non-segmented negative-strand RNA virus of the family Paramyxoviridae. The natural NiV host are fruit bats of the Pteropodidae Family. NiV infection in humans has a range of clinical presentations, from asymptomatic infection to acute respiratory syndrome and fatal encephalitis. About a quarter of the human patients have seizures and about 60% become comatose and might need mechanical ventilation. NiV is also capable of causing disease in pigs and other domestic animals.

The NiV viral envelop contains several membrane proteins, including an envelope protein, F, and an attachment protein, G. The NiV G protein is a Type II membrane protein that facilitates attachment of NiV to host cell membranes. The NiV F protein is a Type I membrane protein that binds to a host cell receptor and facilitates fusion of host and viral membranes. NiV F is a class I fusion protein initially expressed as a single polypeptide precursor, designated F₀. F₀ trimerizes in the endoplasmic reticulum and is processed by a cellular protease at a conserved site generating, F₁ and F₂ polypeptides. The F₂ polypeptide originates from the N-terminal portion of the F₀ precursor and links to the F₁ polypeptide via disulfide bonds. The F₁ polypeptide anchors the mature F protein in the membrane via a transmembrane domain, which is linked to a cytoplasmic tail. Three protomers of the F₂-F₁ heterodimer assemble to form a mature F protein, which adopts a metastable “prefusion” conformation that is triggered to undergo a conformational change that fuses the viral and target-cell membranes.

Although NiV is known to contribute to human illness and disease burden, a vaccine for this virus is not available.

### SUMMARY

Disclosed herein are recombinant NiV F ectodomain trimers comprising protomers comprising one or more modifications (such as amino acid substitutions) that stabilize the F ectodomain trimer in its prefusion conformation. Embodiments of such prefusion-stabilized NiV F ectodomain trimers are demonstrated to produce a superior immune response in animal models compared to corresponding NiV F ectodomain trimers that are not stabilized in the prefusion conformation.

In some embodiments, the recombinant NiV F ectodomain trimer comprises protomers comprising one or more amino acid substitutions that stabilize the NiV F ectodomain trimer in a prefusion conformation, wherein the one or more amino acid substitutions comprise one or more of the following: cysteine substitutions at NiV F positions 104 and 114 (such as L104C and I114C substitutions) that form a non-natural intra-protomer disulfide bond or cysteine substitutions at NiV F positions 114 and 426 (such as I114C and I426C substitutions) that form a non-natural intra-protomer disulfide bond, a proline substitution at NiV F position 191 (such as a S191P substitution), a phenylalanine substitution at NiV F position 172 (such as a L172F substitution), a glycine substitution at NiV F position 70 (such as a Q70G substitution), and a deletion of NiV F positions 102-113 with positions 101 and 114 linked by a glycine-serine linker (such as a (HDLVGDVRLAGV)102-113(GSG) substitution). In a non-limiting embodiment, the one or more amino acid substitutions comprising the cysteine substitutions at NiV F positions 104 and 114 that form a non-natural intra-protomer disulfide bond, the proline substitution at NiV F position 191, and the phenylalanine substitution at NiV F position 172.

In some embodiments, a C-terminal residue of the protomers of the recombinant NiV F ectodomain trimer (such as a residue of the stem region of the trimer) is linked to a trimerization domain (such as GCN4 trimerization domain or a T4 fibritin trimerization domain) to promote trimerization of the ectodomain. In some embodiments, immunogen is soluble. In other embodiments, a C-terminal residue of the protomers of the recombinant NiV F ectodomain trimer (such as a residue of the stem region of the trimer) is linked to a transmembrane domain for membrane anchored forms of the NiV F ectodomain trimer.

In some embodiments, the recombinant NiV F ectodomain trimer is fused to one or more heterologous proteins. For example, in some embodiments, the protomers of the recombinant NiV F ectodomain trimer are fused to a NiV G ectodomain to provide a NiV F-G chimera. In some embodiments, the NiV F ectodomain trimer is linked to at least three NiV G ectodomains, wherein the NiV G ectodomains are fused, directly or indirectly via peptide linker, to an N-terminus of protomers of the recombinant NiV F ectodomain trimer and/or to a C-terminus of a trimerization domain fused to the C-terminus of protomers of the recombinant NiV F ectodomain trimer. In some embodiments, the trimerization domain comprises, for example, a GCN4 trimerization domain, a T4 fibritin trimerization domain, or a GCN4 trimerization domain and a T4 fibritin trimerization domain.

In some embodiments, the protomers of the recombinant NiV F ectodomain trimer further comprise one or more additional mutations, such as amino acid substitutions that stabilize the recombinant NiV F ectodomain trimer in the prefusion conformation, or amino acid substitutions to inhibit or prevent protease cleavage at a F₁/F₂ protease cleavage site of the F ectodomain.

In some embodiments, the recombinant NiV F ectodomain trimer can be included on a protein nanoparticle, such as a ferritin protein nanoparticle.

In some embodiments, an immunogen is provided that comprises a trimer of fusion proteins, each fusion protein comprising, in an N- to C-terminal direction: one or more NiV G ectodomains and a trimerization domain; a trimerization domain and one or more NiV G ectodomains; or one or more NiV G ectodomains, a trimerization domain, and one or more NiV G ectodomains.

In some embodiments, a protein nanoparticle, such as a ferritin nanoparticle, is provided that comprises a monomeric NiV G ectodomain.

Nucleic acid molecules encoding the disclosed proteins are also provided. For example, a nucleic acid molecule encoding a protomer of a disclosed recombinant NiV F ectodomain trimer stabilized in a prefusion conformation, a chimera of recombinant NiV F ectodomain trimer stabilized in a prefusion conformation and one or more G ectodomains, a multimer of NiV G ectodomains, or a self-assembling protein nanoparticle containing recombinant NiV F ectodomain trimer stabilized in a prefusion conformation or a NiV G ectodomains are also provided, as are vectors including the nucleic acid molecules, and methods of their production.

Immunogenic compositions including a disclosed immunogen that are suitable for administration to a subject are also provided, and may also be contained in a unit dosage form. The immunogen may also contain a carrier to facilitate presentation to the immune system.

Methods of inducing an immune response in a subject are disclosed, as are methods of treating, inhibiting or preventing a NiV infection in a subject, by administering to the subject an effective amount of a disclosed immunogen, nucleic acid molecule, or vector.

The foregoing and other features and advantages of this disclosure will become more apparent from the following detailed description of several embodiments which proceeds with reference to the accompanying figures.

### BRIEF DESCRIPTION OF THE FIGURES

FIGS. 1A-1F show negative stain electron microscopy (EM) images and ribbon diagrams for the NiV05 (FIG. 1A), NiV08 (FIG. 1B), NiV09 (FIG. 1C), NiV14 (FIG. 1D), NiV15 (FIG. 1E), and NiV06 (FIG. 1F) NiV F ectodomain trimers.

FIG. 2 shows negative stain EM images for the NiVop08 NiV F ectodomain trimer alone or in complex with the 5B3 Fab, which targets the prefusion conformation of NiV F.

FIGS. 3A-3C show a schematic diagram (FIG. 3A) and results (FIGS. 3B-3D) for an in vivo immunization assay. FIG. 3B, Sera from immunized mice was assessed for binding to prefusion NiV F probe (NiVop08 ectodomain trimer) and postfusion NiV F probe (NiV06 ectodomain trimer) by Octet binding assay. FIG. 3C, Sera from mice immunized with the indicated immunogens was assessed for NiV neutralization.

FIG. 4 shows negative stain EM for NiV G ectodomain multimers having a format of G-T4 fibritin trimerization domain (G-Fd) or G-T4 fibritin trimerization domain-G (G-Fd-G).

FIGS. 5A-5C show negative stain EM images and ribbon diagrams for self-assembled ferritin nanoparticles containing the NiV G ectodomain linked to ferritin by a 5 amino acid peptide linker (FIG. 5A), a 15 amino acid peptide linker (FIG. 5B), or a 25 amino acid peptide linker (FIG. 5C).

FIGS. 6A-6C show a schematic diagram (FIG. 6A) and results (FIGS. 6B-6C) for an in vivo immunization assay of the NiV G ectodomain multimers and NiV G ectodomain-containing ferritin nanoparticles. FIG. 3B, Sera from immunized mice was assessed for binding to monovalent NiV G probe by Octet binding assay. FIG. 3C, Sera from mice immunized with the indicated immunogens was assessed for NiV neutralization.

FIGS. 7A-7C show a schematic diagram (FIG. 7A) and negative stain EM images (FIGS. 7B and 7C) for chimeric NiV F-G constructs containing a NiV F ectodomain trimer and three monomeric NiV G ectodomains.

FIGS. 8A-8E show a schematic diagram (FIG. 8A) and results (FIGS. 8B-6E) for an in vivo immunization assay of the NiV F-G chimeras. Sera from immunized mice was assessed for binding to a prefusion NiV F ectodomain trimer (FIG. 8B), a postfusion NiV F ectodomain trimer (FIG. 8C), or a NiV G ectodomain monomer (FIG. 8D) by Octet binding assay. FIG. 8E, Sera from mice immunized with the indicated immunogens was assessed for NiV neutralization.

FIGS. 9A-9D show an immunization protocol and schematic diagram (FIGS. 9A and 9B) and results (FIGS. 9C-9D) for an in vivo immunization assay of the NiV F, G, and F-G chimeric immunogens in a ferret model. The animals were immunized with the preF, postF, G hexamer, or preF/G chimera immunogen in 16 different groups. Sera collected from immunized animals at week six (FIG. 9C) and nine (FIG. 9D) assessed for NiV neutralization using the pseudovirus neutralization assay described above.

FIG. 10 shows results for a virus neutralization test (VNT) using live NiV infection of cells in vitro performed with sera from the 10 μg and 100 μg mRNA immunization conditions with the preF, preF/G chimera, and G-hexamer immunogens.

FIG. 11 is a graph illustrating the correlation of neutralization of NiV pseudovirus and live NiV.

FIGS. 12A-12C show an immunization protocol (FIG. 12A) and results (FIGS. 12B and 12C) for an in vivo immunization assay of NiV Pre-F, Post-F, WT-F, G-hexamer, and G-tetramer (+stalk) immunogens in a mouse model, with several variations, including mRNA or protein-based immunization, signal sequence, and soluble or membrane-anchored immunogen.

FIGS. 12A-12C show an immunization protocol (FIG. 12A) and results (FIGS. 12B and 12C) for an in vivo immunization assay of NiV Pre-F, Post-F, WT-F, G-hexamer, and G-tetramer (+stalk) immunogens in a mouse model, with several variations, including mRNA or protein-based immunization, signal sequence, and soluble or membrane-anchored immunogen.

FIGS. 13A-13D show an immunization protocol and schematic diagram (FIGS. 13A and 13B) and results (FIGS. 13C-13D) for an in vivo immunization assay of NiV F, G, and F/G chimeric immunogens in a mouse model. The animals were immunized with the preF, postF, G-hexamer, or preF/G chimera immunogen in different groups using protein or mRNA based immunization systems (FIG. 13B). Sera collected from immunized animals at week six was assessed for preF-binding IgG (FIG. 13C) and G-binding IgG (FIG. 13D).

### SEQUENCES

The nucleic and amino acid sequences listed herein are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:
SEQ ID NO: 1 is an amino acid sequence including NiV01 protein.
SEQ ID NO: 2 is an amino acid sequence including NiV02 protein.
SEQ ID NO: 3 is an amino acid sequence including NiV03 protein.
SEQ ID NO: 4 is an amino acid sequence including NiV04 protein.
SEQ ID NO: 5 is an amino acid sequence including NiV05 protein.
SEQ ID NO: 6 is an amino acid sequence including NiV06 protein.
SEQ ID NO: 7 is an amino acid sequence including NiV07 protein.
SEQ ID NO: 8 is an amino acid sequence including NiV08 protein.
SEQ ID NO: 9 is an amino acid sequence including NiV09 protein.
SEQ ID NO: 10 is an amino acid sequence including NiV10 protein.
SEQ ID NO: 11 is an amino acid sequence including NiV11 protein.
SEQ ID NO: 12 is an amino acid sequence including NiV12 protein.
SEQ ID NO: 13 is an amino acid sequence including NiV13 protein.
SEQ ID NO: 14 is an amino acid sequence including NiV14 protein.
SEQ ID NO: 15 is an amino acid sequence including NiV15 protein.
SEQ ID NO: 16 is an amino acid sequence including NiV16 protein.
SEQ ID NO: 17 is an amino acid sequence including NiVop01 protein.
SEQ ID NO: 18 is an amino acid sequence including NiVop02 protein.
SEQ ID NO: 19 is an amino acid sequence including NiVop03 protein.
SEQ ID NO: 20 is an amino acid sequence including NiVop04 protein.
SEQ ID NO: 21 is an amino acid sequence including NiVop05 protein.
SEQ ID NO: 22 is an amino acid sequence including NiVop06 protein.
SEQ ID NO: 23 is an amino acid sequence including NiVop07 protein.
SEQ ID NO: 24 is an amino acid sequence including NiVop08 protein.
SEQ ID NO: 25 is an amino acid sequence including NiVop09 protein.
SEQ ID NO: 26 is an amino acid sequence including NiVop12 protein.
SEQ ID NO: 27 is an amino acid sequence including NiVop13 protein.
SEQ ID NO: 28 is an amino acid sequence including NiVop14 protein.
SEQ ID NO: 29 is an amino acid sequence including NiVop15 protein.
SEQ ID NO: 30 is an amino acid sequence including NiVop16 protein.
SEQ ID NO: 31 is an amino acid sequence including NiVop17 protein.
SEQ ID NO: 32 is an amino acid sequence including NiVop18 protein.
SEQ ID NO: 33 is an exemplary nucleic acid sequence encoding full-length NiV F with NiVop08 substitutions.
SEQ ID NO: 34 is an amino acid sequence including Fd-G protein.
SEQ ID NO: 35 is an amino acid sequence including Fd-GG protein.
SEQ ID NO: 36 is an amino acid sequence including Fd-GGG protein.
SEQ ID NO: 37 is an amino acid sequence including G-Fd-G protein (soluble G-hexamer).
SEQ ID NO: 38 is an amino acid sequence including NiV G linked to a ferritin subunit by a five amino acid linker (G-ln5-Fer).
SEQ ID NO: 39 is an amino acid sequence including NiV G linked to a ferritin subunit by a 15 amino acid linker (G-ln15-Fer).
SEQ ID NO: 40 is an amino acid sequence including NiV G linked to a ferritin subunit by a 25 amino acid linker (G-ln25-Fer).
SEQ ID NO: 41 is an amino acid sequence including NiV G linked to a ferritin subunit by a 35 amino acid linker (G-ln35-Fer).
SEQ ID NO: 42 is an amino acid sequence including NiV G linked to a lumazine synthase subunit (G-LS).
SEQ ID NO: 43 is an amino acid sequence including NiVop08 linked to NiV G by GCN4 and Fd trimerization domains (NiVop08-TD-G).
SEQ ID NO: 44 is an amino acid sequence including NiV G linked to NiVop09 linked to GCN4 and Fd trimerization domains (G-NiVop08-TD).
SEQ ID NO: 45 is an amino acid sequence including NiVop06 linked to NiV G by GCN4 and Fd trimerization domains (NiV06-TD-G).
SEQ ID NO: 46 is an amino acid sequence including NiVop06 linked to two copies of NiV G by GCN4 and Fd trimerization domains (NiV06-TD-GG).
SEQ ID NO: 47 is an amino acid sequence including NiVop08 linked to two copies of NiV G by GCN4 and Fd trimerization domains (NiVop08-TD-GG).
SEQ ID NO: 48 is an amino acid sequence including NiVop06 linked to three copies of NiV G by GCN4 and Fd trimerization domains (NiV06-TD-GGG).
SEQ ID NO: 49 is an amino acid sequence including NiVop08 linked to three copies of NiV G by GCN4 and Fd trimerization domains (NiVop08-TD-GGG).
SEQ ID NO: 50 is an amino acid sequence including NiV G linked to NiV06 linked to GCN4 and Fd trimerization domains (G-NiV06-TD).
SEQ ID NO: 51 is an amino acid sequence including NIV G linked to NiVop08 linked to GCN4 and Fd trimerization domains (G-NiVop08-TD).
SEQ ID NO: 52 is an exemplary sequence of a wild-type NiV F protein.
SEQ ID NOs: 53 and 54 are exemplary sequences of wild-type NiV G proteins.
SEQ ID NOs: 55-58 are amino acid sequences of protein nanoparticle subunits.
SEQ ID NO: 59 is an amino acid sequence including NiVop08 linked to NiV G by a GCN4 trimerization domain (NiVop08-GCN4-G).
SEQ ID NO: 60 is an amino acid sequence including NiVop08 linked to NiV G by a Fd trimerization domains (NiVop08-Fd-G).
SEQ ID NO: 61 is an exemplary nucleic acid sequence encoding NiVop08-TD-G.
SEQ ID NO: 62 is an exemplary nucleic acid sequence encoding G-NiVop08-TD.
SEQ ID NO: 63 is an exemplary nucleic acid sequence encoding NiV08.
SEQ ID NO: 64 is an exemplary nucleic acid sequence encoding G-ln5-Ferritin.
SEQ ID NO: 65 is an exemplary nucleic acid sequence encoding NiVop08-GCN4-G.
SEQ ID NO: 66 is an exemplary nucleic acid sequence encoding NiVop08-Fd-G.
SEQ ID NO: 67 is an exemplary nucleic acid sequence encoding G-Fd-G.
SEQ ID NO: 68 is an exemplary sequence of a HeV G protein.
SEQ ID NOs: 69 and 70 are sequences of chimeric proteins containing NiVop8 and HeV G.

### DETAILED DESCRIPTION

Disclosed herein are recombinant NiV F ectodomain trimers comprising protomers comprising one or more modifications (such as amino acid substitutions) that stabilize the F ectodomain trimer in its prefusion conformation. Additionally, provided are chimeras of the recombinant NiV F ectodomain trimer stabilized in the prefusion conformation and one or more G ectodomains, a multimer of NiV G ectodomains, and self-assembling protein nanoparticles containing the recombinant NiV F ectodomain trimer stabilized in the prefusion conformation or a NiV G ectodomain.

Embodiments of the prefusion-stabilized NiV F ectodomain trimer are demonstrated to produce a superior immune response in an animal model compared to corresponding NiV F ectodomain trimers that are not stabilized in the prefusion conformation. Several prefusion-stabilized NiV F ectodomain designs provide a surprisingly good combination of stability, homogeneity, yield, and immunogenicity.

Similarly, embodiments of the disclosed chimeras of the recombinant NiV F ectodomain trimer stabilized in the prefusion conformation and one or more G ectodomains provide an surprisingly good combination of stability, homogeneity, yield, and immunogenicity, particularly given the chimeric aspect of these constructs.

### I. Summary of Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references. As used herein, the singular forms “a,” “an,” and “the,” refer to both the singular as well as plural, unless the context indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. To facilitate review of the various embodiments, the following explanations of terms are provided:

Adjuvant: A vehicle used to enhance antigenicity. In some embodiments, an adjuvant includes a suspension of minerals (alum, aluminum hydroxide, or phosphate) on which antigen is adsorbed; or water-in-oil emulsion, for example, in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). In some embodiments, the adjuvant used in a disclosed immunogenic composition is a combination of lecithin and carbomer homopolymer (such as the ADJUPLEX™ adjuvant available from Advanced BioAdjuvants, LLC, see also Wegmann, Clin Vaccine Immunol, 22(9): 1004-1012, 2015). Additional adjuvants for use in the disclosed immunogenic compositions include the QS21 purified plant extract, Matrix M, AS01, MF59, and ALFQ adjuvants. Immunostimulatory oligonucleotides (such as those including a CpG motif) can also be used as adjuvants. Adjuvants include biological molecules (a “biological adjuvant”), such as costimulatory molecules. Exemplary adjuvants include IL-2, RANTES, GM-CSF, TNF-α, IFN-γ, G-CSF, LFA-3, CD72, B7-1, B7-2, OX-40L, 4-1BBL, immune stimulating complex (ISCOM) matrix, and toll-like receptor (TLR) agonists, such as TLR-9 agonists, Poly I:C, or PolyICLC. (Scc, e.g., Singh (cd.) Vaccine Adjuvants and Delivery Systems. Wiley-Interscience, 2007).

Administration: The introduction of a composition into a subject by a chosen route. Administration can be local or systemic. For example, if the chosen route is intranasal, the composition (such as a composition including a disclosed recombinant NiV F ectodomain) is administered by introducing the composition into the nasal passages of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Amino acid substitution: The replacement of an amino acid in a polypeptide with one or more different amino acids. In the context of a protein sequence, an amino acid substitution is also referred to as a mutation.

Antibody: An immunoglobulin, antigen-binding fragment, or derivative thereof, that specifically binds and recognizes an analyte (antigen) such as NiV F protein, an antigenic fragment thereof, or a dimer or multimer of the antigen. The term “antibody” is used herein in the broadest sense and encompasses various antibody structures, including but not limited to monoclonal antibodies, polyclonal antibodies, multispecific antibodies (e.g., bispecific antibodies), and antibody fragments, so long as they exhibit the desired antigen-binding activity. Non-limiting examples of antibodies include, for example, intact immunoglobulins and variants and fragments thereof that retain binding affinity for the antigen. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies (sec, e.g., Kontermann and Dubel (Ed), Antibody Engineering, Vols. 1-2, 2^{nd} Ed., Springer Press, 2010).

Carrier: An immunogenic molecule to which an antigen can be linked. When linked to a carrier, the antigen may become more immunogenic. Carriers are chosen to increase the immunogenicity of the antigen and/or to elicit antibodies against the carrier which are diagnostically, analytically, and/or therapeutically beneficial. Useful carriers include polymeric carriers, which can be natural (for example, proteins from bacteria or viruses), semi-synthetic or synthetic materials containing one or more functional groups to which a reactant moiety can be attached.

Cavity-filling amino acid substitution: An amino acid substitution that fills a cavity within the protein core of a protein, such as a NiV F ectodomain. Cavities are essentially voids within a folded protein where amino acids or amino acid side chains are not present. In several embodiments, a cavity filling amino acid substitution is introduced to fill a cavity present in the prefusion conformation of the NiV F ectodomain core that collapses (e.g., has reduced volume) after transition to the postfusion conformation.

Conservative variants: “Conservative” amino acid substitutions are those substitutions that do not substantially affect or decrease a function of a protein, such as the ability of the protein to induce an immune response when administered to a subject. The term conservative variation also includes the use of a substituted amino acid in place of an unsubstituted parent amino acid. Furthermore, individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of the recombinant NiV F ectodomain trimer, such as the ability to induce an immune response when administered to a subject. For instance, if an amino acid residue is essential for a function of the protein, even an otherwise conservative substitution may disrupt that activity. Thus, a conservative substitution does not alter the basic function of a protein of interest.

Control: A reference standard. In some embodiments, the control is a negative control sample obtained from a healthy patient. In other embodiments, the control is a positive control sample obtained from a patient diagnosed with NiV infection. In still other embodiments, the control is a historical control or standard reference value or range of values (such as a previously tested control sample, such as a group of NiV patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Degenerate variant: In the context of the present disclosure, a “degenerate variant” refers to a polynucleotide encoding a polypeptide that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

Effective amount: An amount of agent, such as an immunogen, that is sufficient to elicit a desired response, such as an immune response in a subject. It is understood that to obtain a protective immune response against an antigen of interest can require multiple administrations of a disclosed immunogen, and/or administration of a disclosed immunogen as the “prime” in a prime boost protocol wherein the boost immunogen can be different from the prime immunogen. Accordingly, an effective amount of a disclosed immunogen can be the amount of the immunogen sufficient to elicit a priming immune response in a subject that can be subsequently boosted with the same or a different immunogen to elicit a protective immune response.

In one example, a desired response is to inhibit or reduce or prevent NiV infection. The NiV infection does not need to be completely eliminated or reduced or prevented for the method to be effective. For example, administration of an effective amount of the agent can decrease the NiV infection (for example, as measured by infection of cells, or by number or percentage of subjects infected by NiV) by a desired amount, for example by at least 50%, at least 60%, at least 70%, at least 80%, at least 90%, at least 95%, at least 98%, or even at least 100% (elimination or prevention of detectable NiV infection), as compared to a suitable control.

Expression: Transcription or translation of a nucleic acid sequence. For example, a gene is expressed when its DNA is transcribed into an RNA or RNA fragment, which in some examples is processed to become mRNA. A gene may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. The term “expression” is used herein to denote either transcription or translation. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

GCN4 trimerization domain: A trimerization domain from the GCN4 protein that comprises a leucine zipper amino acid sequence that naturally forms a trimeric structure. Embodiments of the GCN4 trimerization domain is described, for example, Harbury et al. (1993 Science 262:1401-1407). In some examples, a GCN4 trimerization domain can be included in the amino acid sequence of a disclosed recombinant protein so that the recombinant protein will trimerize. A non-limiting example of a GCN4 trimerization domain sequence for use with the disclosed embodiments is provided as KLMKQIEDKIEEILSKIYHIENEIARIKKLIGEAP (residues 485-519 of SEQ ID NO: 1).

Heterologous: Originating from a different genetic source.

Host cells: Cells in which a vector can be propagated and its nucleic acid expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Immune response: A response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies.

Immunogen: A compound, composition, or substance (for example, a recombinant NiV F ectodomain trimer) that can elicit an immune response in an animal, including compositions that are injected or absorbed into an animal. Administration of an immunogen to a subject can lead to protective immunity against a pathogen of interest.

Immunogenic composition: A composition comprising a disclosed recombinant NiV F ectodomain trimer that induces a measurable CTL response against the NiV, or induces a measurable B cell response (such as production of antibodies) against the NiV, when administered to a subject. It further refers to isolated nucleic acid molecules and vectors encoding a protomer of a disclosed recombinant NiV F ectodomain trimer that can be used to express the protomer (and thus be used to elicit an immune response against recombinant NiV F ectodomain trimer). For in vivo use, the immunogenic composition will typically include the recombinant NiV F ectodomain trimer or a nucleic acid molecule encoding a protomer of the recombinant NiV F ectodomain trimer in a pharmaceutically acceptable carrier and may also include other agents, such as an adjuvant.

Inhibiting or treating a disease: Inhibiting the full development of a disease or condition, for example, in a subject who is at risk for a disease such as NiV infection. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

Isolated: An “isolated” biological component has been substantially separated or purified away from other biological components, such as other biological components in which the component naturally occurs, such as other chromosomal and extrachromosomal DNA, RNA, and proteins. Proteins, peptides, nucleic acids, and viruses that have been “isolated” include those purified by standard purification methods. Isolated does not require absolute purity, and can include protein, peptide, nucleic acid, or virus molecules that are at least 50% isolated, such as at least 75%, 80%, 90%, 95%, 98%, 99%, or even 99.9% isolated.

Linker and Linked: A bi-functional molecule that can be used to link two molecules into one contiguous molecule. Non-limiting examples of peptide linkers include glycine-serine peptide linkers. Unless context indicates otherwise, reference to “linking” a first polypeptide and a second polypeptide, or to two polypeptides “linked” together, or to a first polypeptide having a “linkage” to a second polypeptide, refers to covalent linkage by peptide bond (for example via a peptide linker) such that the first and second polypeptides form a contiguous polypeptide chain. If a peptide linker is involved, the covalent linkage of the first and second polypeptides can be to the N- and C-termini of the peptide linker. Typically, such linkage is accomplished using molecular biology techniques to genetically manipulate DNA encoding the first polypeptide linked to the second polypeptide by the peptide linker.

Native protein, sequence, or disulfide bond: A polypeptide, sequence or disulfide bond that has not been modified, for example, by selective mutation. For example, selective mutation to focus the antigenicity of the antigen to a target epitope, or to introduce a disulfide bond into a protein that does not occur in the native protein. Native protein or native sequence are also referred to as wild-type protein or wild-type sequence. A non-native disulfide bond is a disulfide bond that is not present in a native protein, for example, a disulfide bond that forms in a protein due to introduction of one or more cysteine residues into the protein by genetic engineering.

Nipah Virus (NiV): Nipah henipavirus is an enveloped non-segmented negative-sense single-stranded RNA virus of the family Paramyxoviridae. The NiV genome is ˜18,000 nucleotides in length and includes 6 genes encoding 9 proteins, including the glycoproteins G, and F. Exemplary native NiV strain sequences are known to the person of ordinary skill in the art. Several models of human NiV infection are available, including model organisms infected with NiV, such as ferrets, mice, golden hamsters, guinea pigs, and African Green Monkeys (see, e.g., Geisbert et al., Curr. Top. Microbiol. Immunol., 359:153-77, 2012, which is incorporated by reference herein in its entirety).

The natural NiV host are fruit bats of the Pteropodidae Family. NiV infection in humans has a range of clinical presentations, from asymptomatic infection to acute respiratory syndrome and fatal encephalitis. NiV is also capable of causing disease in pigs and other domestic animals. In humans, NiV infection typically presents as fever, headache and drowsiness. Cough, abdominal pain, nausea, vomiting, weakness, problems with swallowing and blurred vision are relatively common. About a quarter of the human patients have seizures and about 60% become comatose and might need mechanical ventilation. In patients with severe disease, their conscious state may deteriorate and they may develop severe hypertension, fast heart rate, and very high temperature.

NiV attachment glycoprotein (G): An NiV envelope glycoprotein that is a type II membrane protein and facilitates attachment of NiV to host cell membranes. The full-length G protein has an N-terminal cytoplasmic tail and transmembrane domain (CT and TM, approximately amino acids 1-176), and an ectodomain (approximately amino acids 177-602). An exemplary NiV G protein sequence from NiV G from a Malaysian stain is provided herein as SEQ ID NO: 53 (NCBI Reference Sequence NP_112027.1, which is incorporated by reference herein):

MPAENKKVRFENTTSDKGKIPSKVIKSYYGTMDIKKINEGLLDSKILSAF
NTVIALLGSIVIIVMNIMIIQNYTRSTDNQAVIKDALQGIQQQIKGLADK
IGTEIGPKVSLIDTSSTITIPANIGLLGSKISQSTASINENVNEKCKFTL
PPLKIHECNISCPNPLPFREYRPQTEGVSNLVGLPNNICLQKTSNQILKP
KLISYTLPVVGQSGTCITDPLLAMDEGYFAYSHLERIGSCSRGVSKQRII
GVGEVLDRGDEVPSLFMTNVWTPPNPNTVYHCSAVYNNEFYYVLCAVSTV
GDPILNSTYWSGSLMMTRLAVKPKSNGGGYNQHQLALRSIEKGRYDKVMP
YGPSGIKQGDTLYFPAVGFLVRTEFKYNDSNCPITKCQYSKPENCRLSMG
IRPNSHYILRSGLLKYNLSDGENPKVVFIEISDQRLSIGSPSKIYDSLGQ
PVFYQASFSWDTMIKFGDVLTVNPLVVNWRNNTVISRPGQSQCPRFNTCP
EICWEGVYNDAFLIDRINWISAGVFLDSNQTAENPVFTVFKDNEILYRAQ
LASEDTNAQKTITNCFLLKNKIWCISLVEIYDTGDNVIRPKLFAVKIPEQ
CT

An exemplary NiV G protein sequence from NiV G from a Bangladesh stain is provided herein as SEQ ID NO: 54 (GenBank Reference No. AAY43916.1, which is incorporated by reference herein):

MPTESKKVRFENTASDKGKNPSKVIKSYYGTMDIKKINEGLLDSKILSAF
NTVIALLGSIVIIVMNIMIIQNYTRSTDNQAMIKDALQSIQQQIKGLADK
IGTEIGPKVSLIDTSSTITIPANIGLLGSKISQSTASINENVNEKCKFTL
PPLKIHECNISCPNPLPFREYKPQTEGVSNLVGLPNNICLQKTSNQILKP
KLISYTLPVVGQSGTCITDPLLAMDEGYFAYSHLEKIGSCSRGVSKQRII
GVGEVLDRGDEVPSLFMTNVWTPSNPNTVYHCSAVYNNEFYYVLCAVSVV
GDPILNSTYWSGSLMMTRLAVKPKNNGESYNQHQFALRNIEKGKYDKVMP
YGPSGIKQGDTLYFPAVGFLVRTEFKYNDSNCPIAECQYSKPENCRLSMG
IRPNSHYILRSGLLKYNLSDEENSKIVFIEISDQRLSIGSPSKIYDSLGQ
PVFYQASFSWDTMIKFGDVQTVNPLVVNWRDNTVISRPGQSQCPRFNKCP
EVCWEGVYNDAFLIDRINWISAGVFLDSNQTAENPVFTVFKDNEVLYRAQ
LASEDTNAQKTITNCFLLKNKIWCISLVEIYDTGDNVIRPKLFAVKIPEQ
CT

As used herein, NiV G residue positioning is made with reference to the sequence of the set forth as SEQ ID NO: 53.

NiV fusion (F) protein: An envelope glycoprotein of NiV that facilitates fusion of viral and cellular membranes. In nature, the F protein from NiV is initially synthesized as a single polypeptide precursor approximately 550 amino acids in length, designated F₀. F₀ includes an N-terminal signal peptide that directs localization to the endoplasmic reticulum, where the signal peptide is proteolytically cleaved. The remaining F₀ residues oligomerize to form a trimer and may be proteolytically processed by a cellular protease to generate two disulfide-linked fragments, F₁ and F₂. In NiV F the cleavage site is located approximately between residues 109/110. The smaller of these fragments, F₂, originates from the N-terminal portion of the F₀ precursor (approximately residues 25-109). The larger of these fragments, F₁, includes the C-terminal portion of the F₀ precursor (approximately residues 110-550) including an extracellular/lumenal region (approximately residues 110-495), and a transmembrane and cytosolic regions (approximately residues 495-550). The extracellular portion of the NiV F protein is the NiV F ectodomain, which includes the F₂ protein and the F₁ ectodomain. The fusion peptide is located at the N-terminal segment of the F1 ectodomain, at approximately residues 110-122.

The NiV F protein exhibits remarkable sequence conservation within NiV strain. In view of this conservation, the person of ordinary skill in the art can easily compare amino acid positions of different NiV F proteins. Unless context indicates otherwise, the numbering of NiV F amino acids is made with reference to SEQ ID NO: 52 (NCBI Reference Sequence NP_112026.1, which is incorporated by reference herein):

MVVILDKRCYCNLLILILMISECSVGILHYEKLSKIGLVKGVTRKYKIKS
NPLTKDIVIKMIPNVSNMSQCTGSVMENYKTRLNGILTPIKGALEIYKNN
THDLVGDVRLAGVIMAGVAIGIATAAQITAGVALYEAMKNADNINKLKSS
IESTNEAVVKLQETAEKTVYVLTALQDYINTNLVPTIDKISCKQTELSLD
LALSKYLSDLLFVFGPNLQDPVSNSMTIQAISQAFGGNYETLLRTLGYAT
EDFDDLLESDSITGQIIYVDLSSYYIIVRVYFPILTEIQQAYIQELLPVS
FNNDNSEWISIVPNFILVRNTLISNIEIGFCLITKRSVICNQDYATPMTN
NMRECLTGSTEKCPRELVVSSHVPRFALSNGVLFANCISVTCQCQTTGRA
ISQSGEQTLLMIDNTTCPTAVLGNVIISLGKYLGSVNYNSEGIAIGPPVF
TDKVDISSQISSMNQSLQQSKDYIKEAQRLLDTVNPSLISMLSMIILYVL
SIASLCIGLITFISFIIVEKKRNTYSRLEDRRVRPTSSGDLYYIGT

Three NiV F protomers oligomerize in the mature F protein, which adopts a metastable prefusion conformation that is triggered to undergo a conformational change to a postfusion conformation upon contact with a target cell membrane. This conformational change exposes a hydrophobic sequence, known as the fusion peptide, which is located at the N-terminus of the F₁ ectodomain, and which associates with the host cell membrane and promotes fusion of the membrane of the virus, or an infected cell, with the target cell membrane.

An NiV F ectodomain trimer “stabilized in a prefusion conformation” comprises one or more amino acid substitutions, deletions, or insertions compared to a corresponding native NiV F sequence that provide for increased retention of the prefusion conformation compared to NiV F ectodomain trimers formed from a corresponding native NiV F sequence. The “stabilization” of the prefusion conformation can be, for example, energetic stabilization (for example, reducing the energy of the prefusion conformation relative to the postfusion open conformation) and/or kinetic stabilization (for example, reducing the rate of transition from the prefusion conformation to the postfusion conformation). Additionally, stabilization of the NiV F ectodomain trimer in the prefusion conformation can include an increase in resistance to denaturation compared to a corresponding native NiV F sequence. Methods of determining if a NiV F ectodomain trimer is in the prefusion conformation are provided herein, and include (but are not limited to) negative stain electron microscopy and antibody binding assays using a prefusion conformation specific antibody, such as the 5B3 antibody.

NiV F prefusion specific antibody: An antibody that specifically binds to the NiV F protein in a prefusion conformation, but does not specifically bind to the NiV F protein in a postfusion conformation. For example, the 5B3 antibody disclosed in US 2016/0347827 (incorporated by reference herein in its entirety) is a NiV prefusion specific antibody.

Nucleic acid molecule: A polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein-coding regions, in the same reading frame.

Pharmaceutically acceptable carriers: The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical delivery of the disclosed immunogens.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions (such as immunogenic compositions) to be administered can contain minor amounts of non-toxic auxiliary substances, such as wetting or emulsifying agents, preservatives, and pH buffering agents and the like, for example sodium acetate or sorbitan monolaurate. In particular embodiments, suitable for administration to a subject the carrier may be sterile, and/or suspended or otherwise contained in a unit dosage form containing one or more measured doses of the composition suitable to induce the desired immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Polypeptide: Any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

Prime-boost vaccination: An immunotherapy including administration of a first immunogenic composition (the primer vaccine) followed by administration of a second immunogenic composition (the booster vaccine) to a subject to induce an immune response. The primer vaccine and/or the booster vaccine include a vector (such as a viral vector, RNA, or DNA vector) expressing the antigen to which the immune response is directed. The booster vaccine is administered to the subject after the primer vaccine; a suitable time interval between administration of the primer vaccine and the booster vaccine, and examples of such timeframes are disclosed herein. In some embodiments, the primer vaccine, the booster vaccine, or both primer vaccine and the booster vaccine additionally include an adjuvant. In one non-limiting example, the primer vaccine is a DNA-based vaccine (or other vaccine based on gene delivery), and the booster vaccine is a protein subunit or protein nanoparticle based vaccine.

Protein nanoparticle: A self-assembling, multi-subunit, protein-based polyhedron shaped structure. The subunits are each composed of proteins or polypeptides (for example a glycosylated polypeptide), and, optionally of single or multiple features of the following: nucleic acids, prosthetic groups, organic and inorganic compounds. Non-limiting examples of protein nanoparticles include ferritin nanoparticles (see, e.g., Zhang, Y. Int. J. Mol. Sci., 12:5406-5421, 2011, incorporated by reference herein), encapsulin nanoparticles (see, e.g., Sutter et al., Nature Struct. and Mol. Biol., 15:939-947, 2008, incorporated by reference herein), Sulfur Oxygenase Reductase (SOR) nanoparticles (see, e.g., Urich et al., Science, 311:996-1000, 2006, incorporated by reference herein), lumazine synthase nanoparticles (see, e.g., Zhang et al., J. Mol. Biol., 306: 1099-1114, 2001) or pyruvate dehydrogenase nanoparticles (sec, e.g., Izard et al., PNAS 96: 1240-1245, 1999, incorporated by reference herein). Ferritin, encapsulin, SOR, lumazine synthase, and pyruvate dehydrogenase are monomeric proteins that self-assemble into a globular protein complexes that in some cases consists of 24, 60, 24, 60, and 60 protein subunits, respectively. In some examples, ferritin, encapsulin, SOR, lumazine synthase, or pyruvate dehydrogenase monomers are linked to a recombinant NiV F ectodomain and self-assemble into a protein nanoparticle presenting the recombinant NiV F ectodomain trimer or a NiV G ectodomain on its surface, which can be administered to a subject to stimulate an immune response to the antigen.

Recombinant: A recombinant nucleic acid molecule is one that has a sequence that is not naturally occurring, for example, includes one or more nucleic acid substitutions, deletions or insertions, and/or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques.

A recombinant virus is one that includes a genome that includes a recombinant nucleic acid molecule.

A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell, or into the genome of a recombinant virus.

Sequence identity: The similarity between amino acid sequences is expressed in terms of the similarity between the sequences, otherwise referred to as sequence identity. Sequence identity is frequently measured in terms of percentage identity; the higher the percentage, the more similar the two sequences are. Homologs, orthologs, or variants of a polypeptide will possess a relatively high degree of sequence identity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. In the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Variants of a polypeptide are typically characterized by possession of at least about 75%, for example, at least about 80%, 85%, 90%, 91%, 92%, 93%, 94%, 95%, 96%, 97%, 98% or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet.

As used herein, reference to “at least 90% identity” (or similar language) refers to “at least 90%, at least 91%, at least 92%, at least 93%, at least 94%, at least 95%, at least 96%, at least 97%, at least 98%, at least 99%, or even 100% identity” to a specified reference sequence.

Signal Peptide: A short amino acid sequence (e.g., approximately 18-25 amino acids in length) that directs newly synthesized secretory or membrane proteins to and through membranes (for example, the endoplasmic reticulum membrane). Signal peptides are typically located at the N-terminus of a polypeptide and are removed by signal peptidases after the polypeptide has crossed the membrane. Signal peptide sequences typically contain three common structural features: an N-terminal polar basic region (n-region), a hydrophobic core, and a hydrophilic c-region). An exemplary signal peptide sequence is set forth as MYSMQLASCVTLTLVLLVNS (residues 1-20 of SEQ ID NO: 1 (NiV01)

Specifically bind: When referring to the formation of an antibody:antigen protein complex, or a protein:protein complex, refers to a binding reaction which determines the presence of a target protein, peptide, or polysaccharide (for example, a glycoprotein), in the presence of a heterogeneous population of proteins and other biologics. Thus, under designated conditions, a particular antibody or protein binds preferentially to a particular target protein, peptide or polysaccharide (such as an antigen present on the surface of a pathogen, for example, an antigenic site at the membrane distal apex of the NiV F ectodomain timer) and does not bind in a significant amount to other proteins or polysaccharides present in the sample or subject. Specific binding can be determined by methods known in the art. A first protein or antibody specifically binds to a target protein when the interaction has a K_{D} of less than 10⁻⁶ Molar, such as less than 10⁻⁷ Molar, less than 10⁻⁸ Molar, less than 10⁻⁹, or even less than 10⁻¹⁰ Molar.

Soluble protein: A protein capable of dissolving in aqueous liquid at room temperature and remaining dissolved. The solubility of a protein may change depending on the concentration of the protein in the water-based liquid, the buffering condition of the liquid, the concentration of other solutes in the liquid, for example salt and protein concentrations, and the heat of the liquid. In several embodiments, a soluble protein is one that dissolves to a concentration of at least 0.5 mg/ml in phosphate buffered saline (pH 7.4) at room temperature and remains dissolved for at least 48 hours.

Subject: Living multi-cellular vertebrate organisms, a category that includes human and non-human mammals. In an example, a subject is a human. In a particular example, the subject is a newborn infant. In an additional example, a subject is selected that is in need of inhibiting of a NiV infection. For example, the subject is either uninfected and at risk of NiV infection or is infected in need of treatment.

T4 fibritin trimerization domain: Also referred to as a “foldon” domain, the T4 fibritin trimerization domain comprises an amino acid sequence that naturally forms a trimeric structure. In some examples, a T4 fibritin trimerization domain can be included in the amino acid sequence of a disclosed recombinant protein so that the antigen will form a trimer. In one example, a T4 fibritin trimerization domain comprises the amino acid sequence set forth as (GYIPEAPRDGQAYVRKDGEWVLLSTF (residues 24-49 of SEQ ID NO: 34). Several embodiments include a T4 fibritin trimerization domain that can be cleaved from a purified protein, for example by incorporation of a thrombin cleave site adjacent to the T4 fibritin trimerization domain that can be used for cleavage purposes.

Transmembrane domain: An amino acid sequence that inserts into a lipid bilayer, such as the lipid bilayer of a cell or virus or virus-like particle. A transmembrane domain can be used to anchor an antigen to a membrane. In some examples a transmembrane domain is a NiV F transmembrane domain.

Under conditions sufficient for: A phrase that is used to describe any environment that permits a desired activity.

Vaccine: A preparation of immunogenic material capable of stimulating an immune response, administered for the prevention, amelioration, or treatment of infectious or other types of disease. The immunogenic material may include attenuated or killed microorganisms (such as bacteria or viruses), or antigenic proteins, peptides, or DNA derived from them. A vaccine may include a disclosed immunogen (such as a recombinant NiV F ectodomain trimer or nucleic acid molecule encoding same), a virus, a cell or one or more cellular constituents. Vaccines may elicit both prophylactic (preventative or protective) and therapeutic responses. Methods of administration vary according to the vaccine, but may include inoculation, ingestion, inhalation or other forms of administration. Vaccines may be administered with an adjuvant to boost the immune response. In one specific, non-limiting example, a vaccine prevents and/or reduces the severity of the symptoms associated with NiV infection and/or decreases the viral load compared to a control.

Vector: An entity containing a DNA or RNA molecule bearing a promoter(s) that is operationally linked to the coding sequence of an antigen(s) of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant nucleic acid vectors having at least some nucleic acid sequences derived from one or more viruses.

Virus-like particle (VLP): A non-replicating, viral shell, derived from any of several viruses. VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. Methods for producing particular VLPs are known in the art. The presence of VLPs following recombinant expression of viral proteins can be detected using conventional techniques known in the art, such as by electron microscopy, biophysical characterization, and the like. Further, VLPs can be isolated by known techniques, e.g., density gradient centrifugation and identified by characteristic density banding. Sec, for example, Baker et al. (1991) Biophys. J. 60:1445-1456; and Hagensee et al. (1994) J. Virol. 68:4503-4505; Vincente, J Invertebr Pathol., 2011; Schneider-Ohrum and Ross, Curr. Top. Microbiol. Immunol., 354: 53073, 2012).

### II. Immunogens

### A. Recombinant NiV F Ectodomain Trimers

Recombinant NiV F ectodomain trimers are disclosed herein that are modified from a native form (e.g., by introduction of one or more amino acid substitutions) to be stabilized in a prefusion conformation. As described in the Examples, embodiments of the disclosed NiV F ectodomain trimers have been selected through multiple rounds of structure based design for optimized solubility, stability, expression, and immunogenicity. The recombinant NiV F ectodomain trimers are useful to induce an immune response in a vertebrate animal (such humans) to NiV. Exemplary embodiments are shown to produce a superior immune response in an animal model compared to corresponding NiV F ectodomain trimers that are not stabilized in the prefusion conformation.

In some embodiments, the immunogen comprises a recombinant NiV F ectodomain trimer comprising protomers comprising one or more amino acid substitutions or deletions that stabilize the NiV F ectodomain trimer in the prefusion conformation.

In some embodiments, the protomers of the recombinant NiV F ectodomain trimer comprise cysteine substitutions at NiV F positions 104 and 114 that form a non-natural intra-protomer disulfide bond for stabilization in the prefusion conformation.

In some embodiments, the protomers of the recombinant NiV F ectodomain trimer comprise cysteine substitutions at NiV F positions 104 and 114 that form a non-natural intra-protomer disulfide bond and a proline substitution at NiV F position 191 for stabilization in the prefusion conformation.

In some embodiments, the protomers of the recombinant NiV F ectodomain trimer comprise cysteine substitutions at NiV F positions 104 and 114 that form a non-natural intra-protomer disulfide bond and a phenylalanine substitution at NiV F position 172 for stabilization in the prefusion conformation.

In some embodiments, the protomers of the recombinant NiV F ectodomain trimer comprise cysteine substitutions at NiV F positions 104 and 114 that form a non-natural intra-protomer disulfide bond and a glycine substitution at NiV F position 70 for stabilization in the prefusion conformation.

In some embodiments, the protomers of the recombinant NiV F ectodomain trimer comprise cysteine substitutions at NiV F positions 104 and 114 that form a non-natural intra-protomer disulfide bond, a proline substitution at NiV F position 191, and a phenylalanine substitution at NiV F position 172, for stabilization in the prefusion conformation.

In some embodiments, the protomers of the recombinant NiV F ectodomain trimer comprise cysteine substitutions at NiV F positions 104 and 114 that form a non-natural intra-protomer disulfide bond, a phenylalanine substitution at NiV F position 172, and a glycine substitution at NiV F position 70, for stabilization in the prefusion conformation.

In some embodiments, the protomers of the recombinant NiV F ectodomain trimer comprise cysteine substitutions at NiV F positions 104 and 114 that form a non-natural intra-protomer disulfide bond, a proline substitution at NiV F position 191, and a glycine substitution at NiV F position 70 for stabilization in the prefusion conformation.

In some embodiments, the protomers of the recombinant NiV F ectodomain trimer comprise cysteine substitutions at NiV F positions 104 and 114 that form a non-natural intra-protomer disulfide bond, a proline substitution at NiV F position 191, a phenylalanine substitution at NiV F position 172, and a glycine substitution at NiV F position 70, for stabilization in the prefusion conformation.

In some embodiments, the protomers of the recombinant NiV F ectodomain trimer comprise cysteine substitutions at NiV F positions 114 and 426 that form a non-natural intra-protomer disulfide bond for stabilization in the prefusion conformation.

In some embodiments, the protomers of the recombinant NiV F ectodomain trimer comprise cysteine substitutions at NiV F positions 114 and 426 that form a non-natural intra-protomer disulfide bond and a proline substitution at NiV F position 191 for stabilization in the prefusion conformation.

In some embodiments, the protomers of the recombinant NiV F ectodomain trimer comprise cysteine substitutions at NiV F positions 114 and 426 that form a non-natural intra-protomer disulfide bond and a phenylalanine substitution at NiV F position 172 for stabilization in the prefusion conformation.

In some embodiments, the protomers of the recombinant NiV F ectodomain trimer comprise cysteine substitutions at NiV F positions 114 and 426 that form a non-natural intra-protomer disulfide bond and a glycine substitution at NiV F position 70 for stabilization in the prefusion conformation.

In some embodiments, the protomers of the recombinant NiV F ectodomain trimer comprise cysteine substitutions at NiV F positions 114 and 426 that form a non-natural intra-protomer disulfide bond, a proline substitution at NiV F position 191, and a phenylalanine substitution at NiV F position 172, for stabilization in the prefusion conformation.

In some embodiments, the protomers of the recombinant NiV F ectodomain trimer comprise cysteine substitutions at NiV F positions 114 and 426 that form a non-natural intra-protomer disulfide bond, a phenylalanine substitution at NiV F position 172, and a glycine substitution at NiV F position 70, for stabilization in the prefusion conformation.

In some embodiments, the protomers of the recombinant NiV F ectodomain trimer comprise cysteine substitutions at NiV F positions 114 and 426 that form a non-natural intra-protomer disulfide bond, a proline substitution at NiV F position 191, and a glycine substitution at NiV F position 70 for stabilization in the prefusion conformation.

In some embodiments, the protomers of the recombinant NiV F ectodomain trimer comprise cysteine substitutions at NiV F positions 114 and 426 that form a non-natural intra-protomer disulfide bond, a proline substitution at NiV F position 191, a phenylalanine substitution at NiV F position 172, and a glycine substitution at NiV F position 70, for stabilization in the prefusion conformation.

In some embodiments, the protomers of the recombinant NiV F ectodomain trimer comprise a proline substitution at NiV F position 191 for stabilization in the prefusion conformation.

In some embodiments, the protomers of the recombinant NiV F ectodomain trimer comprise a phenylalanine substitution at NiV F position 172 for stabilization in the prefusion conformation.

In some embodiments, the protomers of the recombinant NiV F ectodomain trimer comprise a glycine substitution at NiV F position 70 for stabilization in the prefusion conformation.

In some embodiments, the protomers of the recombinant NiV F ectodomain trimer comprise a proline substitution at NiV F position 191 and a phenylalanine substitution at NiV F position 172 for stabilization in the prefusion conformation.

In some embodiments, the protomers of the recombinant NiV F ectodomain trimer comprise a proline substitution at NiV F position 191 and a glycine substitution at NiV F position 70 for stabilization in the prefusion conformation.

In some embodiments, the protomers of the recombinant NiV F ectodomain trimer comprise a phenylalanine substitution at NiV F position 172 and a glycine substitution at NiV F position 70 for stabilization in the prefusion conformation.

In some embodiments, the protomers of the recombinant NiV F ectodomain trimer comprise a proline substitution at NiV F position 191, a phenylalanine substitution at NiV F position 172, and a glycine substitution at NiV F position 70, for stabilization in the prefusion conformation.

Any of the above recombinant NiV F proteins can further comprise modification to eliminate the protease cleavage site between the F₁ and F₂ polypeptides to generate a “single chain” recombinant F protein. For example, except for variants listed above including modification within positions 102-113, any of the above recombinant NiV proteins can comprise deletion of NiV F positions 102-113 with positions 101 and 114 linked by a glycine-serine linker

For the embodiments listed above, non-limiting examples of specific amino acid substitutions include: L104C and I114C substitutions for the cysteine substitutions at NiV F positions 104 and 114; I114C and I426C substitutions for the cysteine substitutions at NiV F positions 114 and 426; a S191P substitution for the proline substitution at NiV F position 191; a L172F substitution for the phenylalanine substitution at NiV F position 172; a Q70G substitution for the glycine substitution at NiV F position 70; and a (HDLVGDVRLAGV)102-113(GSG) substitution for the deletion of NiV F positions 102-113 with positions 101 and 114 linked by a glycine-serine linker.

In several embodiments, the protomers of the recombinant NiV F ectodomain can comprise one or more additional amino acid substitutions, for example, to increase stabilization of the prefusion conformation, or for other purposes, such as to increase solubility or to reduce and unwanted immune response.

The above-listed non-native disulfide bonds stabilize the membrane-distal portion of the NiV F ectodomain in its prefusion conformation. Any of these mutations can be combined with modifications to the membrane proximal portion (such as the stem) of the NiV F ectodomain, for example, to increase trimerization of the ectodomain.

In several embodiments, the N-terminal position of the recombinant F₂ polypeptide in the protomer can be one of NiV F positions 20-30 (such as position 25), and the C-terminal position of the F₁ ectodomain can be from the stem region of the ectodomain, such as one of NiV F positions 475-495 (such as positions 480-490, for example, position 488).

Non-limiting examples of protomers of a NiV F ectodomain trimer including amino acid substitutions for stabilization in the prefusion conformation as well as a C-terminal linkage to a trimerization domain are provided as residues 21-486 of any one of SEQ ID NOs: 5, 7-7, 11-18, 20-21, 23-24, and 26-32, and residues 21-477 of any one of SEQ ID NOs: 19, 22, and 25. In some embodiments, the protomers of the NiV F ectodomain trimer comprise an amino acid sequence at least 90% identical to residues 21-486 of any one of SEQ ID NOs: 5, 7-7, 11-18, 20-21, 23-24, and 26-32, or residues 21-477 of any one of SEQ ID NOs: 19, 22, and 25; wherein the protomers comprise the one or more amino acid substitutions that stabilize the NiV F ectodomain trimer in the profusion conformation. In some embodiments, the protomers of the NiV F ectodomain trimer comprise residues 21-486 of any one of SEQ ID NOs: 5, 7-7, 11-18, 20-21, 23-24, and 26-32, or residues 21-477 of any one of SEQ ID NOs: 19, 22, and 25.

In several embodiments, the recombinant NiV F ectodomain trimer is a soluble protein complex, for example, for use as a recombinant subunit vaccine. In several such embodiments, the protomers of the recombinant NiV F ectodomain trimer can each comprise a C-terminal linkage to a trimerization domain, such as a GCN4 trimerization domain or a T4 fibritin trimerization domain. The trimerization domain promotes trimerization and stabilization of the membrane proximal aspect of the recombinant NiV F ectodomain trimer. For example, a C-terminal residue of the protomers of the recombinant NiV F ectodomain trimer (such as a residue of the stem region of the trimer) can be directly linked to the trimerization domain, or indirectly linked to the trimerization domain via a peptide linker. Exemplary linkers include glycine and glycine-serine linkers. Non-limiting examples of exogenous multimerization domains that promote stable trimers of soluble recombinant proteins include: the GCN4 leucine zipper, a T4 fibritin trimerization domain, the trimerization motif from the lung surfactant protein (Hoppe et al. 1994 FEBS Lett 344:191-195) or collagen (McAlinden et al. 2003 J Biol Chem 278:42200-42207), any of which can be linked to the C-terminus of the protomers of a recombinant NiV F ectodomain to promote trimerization, as long as the recombinant NiV F ectodomain trimer retains the prefusion conformation. In some examples, the protomers of the recombinant NiV F ectodomain trimer can be linked to a NiV trimerization domain, for example, each protomer in the trimer can include a C-terminal linkage to the GCN4 trimerization domain, such as a linkage to any one of NiV F positions 470-490, such as NiV F position 488. In specific examples, the GCN4 fibritin trimerization domain can comprise the amino acid sequence IEDKIEEILSKIYHIENEIARIKKLIGEAP (residues 490-519 of NiV01, SEQ ID NO: 1).

Non-limiting examples of protomers of a NiV F ectodomain trimer including amino acid substitutions for stabilization in the prefusion conformation as well as a C-terminal linkage to a trimerization domain are provided as residues 21-519 of any one of SEQ ID NOs: 5, 7-7, 11-18, 20-21, 23-24, and 26-32, and residues 21-510 of any one of SEQ ID NOs: 19, 22, and 25. In some embodiments, the protomers of the NiV F ectodomain trimer comprise an amino acid sequence at least 90% identical to residues 21-519 of any one of SEQ ID NOs: 5, 7-7, 11-18, 20-21, 23-24, and 26-32, or residues 21-510 of any one of SEQ ID NOs: 19, 22, and 25; wherein the protomers comprise the one or more amino acid substitutions that stabilize the NiV F ectodomain trimer in the prefusion conformation. In some embodiments, the protomers of the NiV F ectodomain trimer comprise residues 21-519 of any one of SEQ ID NOs: 5, 7-7, 11-18, 20-21, 23-24, and 26-32, or residues 21-510 of any one of SEQ ID NOs: 19, 22, and 25.

In some embodiments, the recombinant NiV F ectodomain trimer can be a membrane anchored protein complex, for example, for use in an attenuated virus or virus like particle vaccine. Membrane anchoring can be accomplished, for example, by C-terminal linkage of the protomers of the recombinant NiV F ectodomain trimer to a transmembrane domain and optionally a cytoplasmic tail, such as an NiV F transmembrane domain and cytoplasmic tail. In some embodiments, one or more peptide linkers (such as a gly-ser linker, for example, a 10 amino acid glycine-serine peptide linker can be used to link the protomers of the recombinant NiV F ectodomain trimer to the transmembrane domain. A non-limiting example of a transmembrane domain for use with the disclosed embodiments includes an NiV F transmembrane domain, such as ILYVLSIASLCIGLITFISFIIV (residues 496-518 of SEQ ID NO: 52).

Native NiV F proteins from different NiV strains, as well as nucleic acid sequences encoding such proteins and methods, are known and can be altered using the description provided herein to generate a recombinant NiV F ectodomain trimer.

B. NiV F Ectodomain Trimers linked to a Heterologous Moiety

The recombinant NiV F ectodomain can be derivatized or linked to another molecule (such as another peptide or protein). In general, the recombinant NiV F ectodomain is derivatized such that the binding to broadly neutralizing antibodies to a trimer of the recombinant NiV F protein is not affected adversely by the derivatization or labeling. For example, the recombinant NiV F ectodomain can be functionally linked (by chemical coupling, genetic fusion, non-covalent association or otherwise) to one or more other molecular entities, such as an antibody or protein or detection tag.

In some embodiments, the recombinant NiV F ectodomain trimers are fused to a NiV G ectodomain (such as the ectodomain of the G sequence set forth as SEQ ID NO: 53 or 54). For example, the protomers of the recombinant NiV F ectodomain trimer are each fused to a NiV G protein ectodomain. The fusion can be direct or via a peptide linker. In some embodiments, the NiV G ectodomain can be fused, directly or indirectly via a peptide linker to the N-terminus of the protomers of the NiV F ectodomain trimer. In some embodiments, the NiV G ectodomain can be fused, directly or indirectly via a peptide linker, to the C-terminus of the protomers of the NiV F ectodomain trimer. In some such embodiments, the NiV G ectodomain can be fused, directly or indirectly via a peptide linker, to the C-terminus of a trimerization domain (such as a GCN4 or T4 fibritin trimerization domain) fused to the C-terminus of the protomers of the NiV F ectodomain trimer. In some such embodiments, the protomers of the NiV F ectodomain trimer linked to the trimerization domain and the NiV G ectodomain comprise an amino acid sequence set forth as residues 21-981 of SEQ ID NO: 43 (NiVop08-TD(GCN4-Fd)-G) or residues 27-981 of SEQ ID NO: 44 (G-NiVop08-TD(GCN4-Fd)), residues 21-952 of SEQ ID NO: 59 (NiVop08-GCN4-G), or residues 21-946 of SEQ ID NO: 60 (NiVop08-Fd-G), or an amino acid sequence at least 90% identical to residues 21-981 of SEQ ID NO: 43 (NiVop08-TD(GCN4-Fd)-G), residues 27-981 of SEQ ID NO: 44 (G-NiVop08-TD(GCN4-Fd)), residues 21-952 of SEQ ID NO: 59 (NiVop08-GCN4-G), or residues 21-946 of SEQ ID NO: 60 (NiVop08-Fd-G).

In some embodiments, more than one (such as 2, 3, or 4) NiV G ectodomain is fused to the protomers of the NiV F trimer. For example, a first NiV G ectodomain can be fused, directly or indirectly via a peptide linker, to the N-terminus of the protomers of the NiV F ectodomain trimer, and a second NiV G ectodomain can be fused, directly or indirectly via a peptide linker, to the C-terminus of the protomers of the NiV F ectodomain trimer (or to the C-terminus of a trimerization domain (such as a GCN4 or T4 fibritin trimerization domain) fused to the C-terminus of the protomers of the NiV F ectodomain trimer).

In some embodiments, the recombinant NiV F ectodomain trimers are fused to an ectodomain of a G protein from a heterologous henipavirus, such as Hendra virus (HeV), Cedar virus (CedV), Kumasi virus (KV), Hendra virus (HeV), or Mòjiãng virus (MojV). For example, the recombinant NiV F ectodomain trimers are fused to an HeV G ectodomain comprising the sequence set forth as:

HeV G Ectodomain (SEQ ID NO: 68, from GenBank: AEB21225.1):

REYRPISQGVSDLVGLPNQICLQKTTSTILKPRLISYTLPINTREGVCIT
DPLLAVDNGFFAYSHLEKIGSCTRGIAKQRIIGVGEVLDRGDKVPSMFMT
NVWTPPNPSTIHHCSSTYHEDFYYTLCAVSHVGDPILNSTSWTESLSLIR
LAVRPKSDSGDYNQKYIAITKVERGKYDKVMPYGPSGIKQGDTLYFPAVG
FLPRTEFQYNDSNCPIIHCKYSKAENCRLSMGVNSKSHYILRSGLLKYNL
SLGGDITLQFIEIADNRLTIGSPSKIYNSLGQPVFYQASYSWDTMIKLGD
VDTVDPLRVQWRNNSVISRPGQSQCPRFNVCPEVCWEGTYNDAFLIDRLN
WVSAGVYLNSNQTAENPVFAVFKDNEILYQVPLAEDDTNAQKTITDCFLL
ENVIWCISLVEIYDTGDSVIRPKLFAVKIPAQCSES

In some such embodiments, the protomers of the recombinant NiV F ectodomain trimer are each fused to the ectodomain of the G protein from the henipavirus, such as an HeV G ectodomain. The fusion can be direct or via a peptide linker. In some embodiments, the ectodomain of the G protein from the henipavirus (such as HeV G ectodomain) can be fused, directly or indirectly via a peptide linker to the N-terminus of the protomers of the NiV F ectodomain trimer. In some embodiments, the ectodomain of the G protein from the henipavirus (such as HeV G ectodomain) can be fused, directly or indirectly via a peptide linker, to the C-terminus of the protomers of the NiV F ectodomain trimer. In some such embodiments, the ectodomain of the G protein from the henipavirus (such as HeV G ectodomain) can be fused, directly or indirectly via a peptide linker, to the C-terminus of a trimerization domain (such as a GCN4 or T4 fibritin trimerization domain) fused to the C-terminus of the protomers of the NiV F ectodomain trimer. In some such embodiments, the protomers of the NiV F ectodomain trimer linked to the trimerization domain and the ectodomain of the G protein from the henipavirus (such as HeV G ectodomain) comprise an amino acid sequence set forth as:

NiVop8-HeV G (SEQ ID NO: 69):
mysmqlascvtltlvl1vnsQGILHYEKLSKIGLVKGVTRKYKIKSNPLT
KDIVIKMIPNVSNMSQCTGSVMENYKTRLNGILTPIKGALEIYKNNTHDC
VGDVRLAGVCMAGVAIGIATAAQITAGVALYEAMKNADNINKLKSSIEST
NEAVVKLQETAEKTVYVFTALQDYINTNLVPTIDKIPCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVIISLGKYLGSVNYNSEGIAIGPPVFTDKV
DISSQISSMNQSLQQSKDYIKEAQRLLDTVNPSLKLMKQIEDKIEEILSK
IYHIENEIARIKKLIGEAPGSGYIPEAPRDGQAYVRKDGEWVLLSTFLGS
GGGGGGVSDLVGLPNQICLQKTTSTILKPRLISYTLPINTREGVCITDPL
LAVDNGFFAYSHLEKIGSCTRGIAKQRIIGVGEVLDRGDKVPSMFMTNVW
TPPNPSTIHHCSSTYHEDFYYTLCAVSHVGDPILNSTSWTESLSLIRLAV
RPKSDSGDYNQKYIAITKVERGKYDKVMPYGPSGIKQGDTLYFPAVGFLP
RTEFQYNDSNCPIIHCKYSKAENCRLSMGVNSKSHYILRSGLLKYNLSLG
GDITLQFIEIADNRLTIGSPSKIYNSLGQPVFYQASYSWDTMIKLGDVDT
VDPLRVQWRNNSVISRPGQSQCPRFNVCPEVCWEGTYNDAFLIDRLNWVS
AGVYLNSNQTAENPVFAVFKDNEILYQVPLAEDDTNAQKTITDCFLLENV
IWCISLVEIYDTGDSVIRPKLFAVKIPAQCSESgglvprgshhhhhhsaw
shpqfek

HeV G-NiVop8 (SEQ ID NO: 70):
mysmqlascvtltlvllvnsqrpqtegvsnlvglpnniclqktsnqilkp
klisytlpvvgqsgtcitdpllamdegyfayshlerigscsrgvskqrii
gvgevldrgdevpslfmtnvwtppnpntvyhcsavynnefyyvlcavstv
gdpilnstywsgs1mmtrlavkpksngggynqhqlalrsiekgrydkvmp
ygpsgikqgdtlyfpavgflvrtefkyndsncpitkcqyskpencrlsmg
irpnshyilrsgllkynlsdgenpkvvfieisdqrlsigspskiydslgq
pvfyqasfswdtmikfgdvltvnplvvnwrnntvisrpgqsqcprfntcp
eicwegvyndaflidrinwisagvfldsnqtaenpvftvfkdneilyraq
lasedtnaqktitncfllknkiwcislveiydtgdnvirpklfavkipeq
ctgggQGILHYEKLSKIGLVKGVTRKYKIKSNPLTKDIVIKMIPNVSNMS
QCTGSVMENYKTRLNGILTPIKGALEIYKNNTHDCVGDVRLAGVCMAGVA
IGIATAAQITAGVALYEAMKNADNINKLKSSIESTNEAVVKLQETAEKTV
YVFTALQDYINTNLVPTIDKIPCKQTELSLDLALSKYLSDLLFVFGPNLQ
DPVSNSMTIQAISQAFGGNYETLLRTLGYATEDFDDLLESDSITGQIIYV
DLSSYYIIVRVYFPILTEIQQAYIQELLPVSFNNDNSEWISIVPNFILVR
NTLISNIEIGFCLITKRSVICNQDYATPMTNNMRECLTGSTEKCPRELVV
SSHVPRFALSNGVLFANCISVTCQCQTTGRAISQSGEQTLLMIDNTTCPT
AVLGNVIISLGKYLGSVNYNSEGIAIGPPVFTDKVDISSQISSMNQSLQQ
SKDYIKEAQRLLDTVNPSLKLMKQIEDKIEEILSKIYHIENEIARIKKLI
GEAPGSGYIPEAPRDGQAYVRKDGEWVLLSTFLGSLVPRGSHHHHHHSAW
SHPQFEK

The above sequences include an N-terminal signal peptide, a NiV F ectodomain (NiVop8), a HeV G ectodomain, a GCN4 trimerization domain, a T4-fibritin trimerization domain, a thrombin cleavage site, a HIS tag and a Strep tag, as well as various linker residues between segments. Purified forms of these proteins typically lack the N-terminal signal peptide and C-terminal residues removed by thrombin cleavage.

In some embodiments, more than one (such as 2, 3, or 4) ectodomain of the G protein from the henipavirus (such as HeV G ectodomain) is fused to the protomers of the NiV F trimer. For example, a first the ectodomain of the G protein from the henipavirus (such as HeV G ectodomain) can be fused, directly or indirectly via a peptide linker, to the N-terminus of the protomers of the NiV F ectodomain trimer, and a second the ectodomain of the G protein from the henipavirus (such as HeV G ectodomain) can be fused, directly or indirectly via a peptide linker, to the C-terminus of the protomers of the NiV F ectodomain trimer (or to the C-terminus of a trimerization domain (such as a GCN4 or T4 fibritin trimerization domain) fused to the C-terminus of the protomers of the NiV F ectodomain trimer).

### C. NiV G Multimers Including a Trimerization Domain

In some embodiments, an immunogen is provided that comprises a multimer of NiV G ectodomains. In some embodiments, the immunogen comprises a trimer of fusion proteins, each fusion protein comprising one or more NiV G ectodomains and a trimerization domain (such as a GCN4 trimerization domain, a T4 fibritin trimerization domain, or a GCN4 trimerization domain fused to a T4 fibritin trimerization domain). In some embodiments, the fusion protein comprises, in an N- to C-terminal direction, one or more (such as one, two, or three) NiV G ectodomains and a trimerization domain. In some embodiments, the fusion protein comprises, in an N- to C-terminal direction, a trimerization domain and one or more (such as one, two, or three) NiV G ectodomains. In some embodiments, the fusion protein comprises, in an N- to C-terminal direction, one or more (such as one, two, or three) NiV G ectodomains, a trimerization domain, and one or more (such as one, two, or three) NiV G ectodomains. The trimerization domains interact to form the trimer. In some embodiment, the fusion proteins in the trimer comprise or consist of an amino acid sequence set forth as residues 21-463 of SEQ ID NO: 34, residues 21-895 of SEQ ID NO: 35, residues 21-1327 of SEQ ID NO: 36, residues 23-912 of SEQ ID NO: 37, or an sequence at least 90% identical to any one of residues 21-463 of SEQ ID NO: 34, residues 21-895 of SEQ ID NO: 35, residues 21-1327 of SEQ ID NO: 36, or residues 23-912 of SEQ ID NO: 37.

### D. Additional Description

The protomers in the recombinant NiV F ectodomain trimer can comprise modifications of the native NiV F sequence in addition to those noted above, such as amino acid substitutions, deletions or insertions, glycosylation and/or covalent linkage to unrelated proteins (e.g., a protein tag), as long as the recombinant NiV F ectodomain trimer remains stabilized in the prefusion conformation and retains immunogenicity. Further, the fusion proteins in the NiV G ectodomain multimer can comprise modifications of the native NiV G sequence, such as amino acid substitutions, deletions or insertions, glycosylation and/or covalent linkage to unrelated proteins (e.g., a protein tag), as long as the NiV G ectodomain retains immunogenicity. These variations in sequence can be naturally occurring variations or they can be engineered through the use of genetic engineering technique known to those skilled in the art. Examples of such techniques are found in sec, e.g., Sambrook et al. (Molecular Cloning: A Laboratory Manual, 4^{th} ed., Cold Spring Harbor, New York, 2012) and Ausubel et al. (In Current Protocols in Molecular Biology, John Wiley & Sons, New York, through supplement 104, 2013, both of which are incorporated herein by reference in their entirety.

In some embodiments, the protomers in the recombinant NiV F ectodomain trimer or the NiV G multimer can comprise one or more amino acid substitutions compared to a corresponding native NiV F or G sequence. For example, in some embodiments, the F₂ polypeptide, F₁ ectodomain, or both, can include up to 20 (such as up to 1, 2, 3, 4, 5, 6, 7, 8, 9, 10, 11, 12, 13, 14, 15, 16, 17, 18, or 19) amino acid substitutions (such as conservative amino acid substitutions) compared to a native NiV For G sequence. The simplest modifications involve the substitution of one or more amino acids for amino acids having similar biochemical properties, such as conservative amino acid substitutions. Such substitutions are likely to have minimal impact on the activity of the resultant protein.

In some embodiments, protomers in the recombinant NiV F ectodomain trimer or the NiV G multimer can be joined at either end to other unrelated sequences (for example non-NiV For G protein sequences, non-viral envelope, or non-viral protein sequences)

In several embodiments, the recombinant NiV F ectodomain trimer or NiV G multimer is soluble in aqueous solution. In some embodiments, the recombinant NiV F ectodomain trimer or NiV G multimer dissolves to a concentration of at least 0.5 mg/ml (such as at least 1.0 mg/ml, 1.5 mg/ml, 2.0 mg/ml, 3.0 mg/ml, 4.0 mg/ml or at least 5.0 mg/ml) in aqueous solution (such as phosphate buffered saline (pH 7.4) or 350 mM NaCl (pH 7.0)) at room temperature (e.g., 20-22 degrees Celsius) and remain dissolved for at least 12 hours (such as at least 24 hours, at least 48 hours, at least one week, at least two weeks, at least one month, or more time). In one embodiment, the phosphate buffered saline includes NaCl (137 mM), KCl (2.7 mM), Na₂HPO₄ (10 mM), KH₂PO₄ (1.8 mM) at pH 7.4. In some embodiments, the phosphate buffered saline further includes CaCl₂) (1 mM) and MgCl₂ (0.5 mM). The person of skill in the art is familiar with methods of determining if a protein remains in solution over time. For example, the concentration of the protein dissolved in an aqueous solution can be tested over time using standard methods.

In some embodiments, the recombinant NiV F ectodomain trimer can be provided as a homogenous population of soluble trimers that does not include detectable NiV F ectodomain trimer in a postfusion conformation. The conformation of the NiV F ectodomain trimer can be detected, for example, by negative stain electron microscopy and/or specific binding by appropriate pre- or post-fusion specific antibody. In some embodiments, at least about 95% of the recombinant NiV F ectodomain trimer (such as at least about 95%, 96%, 97%, 98%, 99% or 99.9% of the NiV F proteins) in the homogeneous population are stabilized in the prefusion conformation.

In some embodiments, the recombinant NiV F ectodomain trimer retains specific binding for a prefusion specific antibody following incubation at 50° C. for one hour in phosphate buffered saline. In some embodiments, the recombinant NiV F ectodomain trimer retains specific binding for a prefusion specific antibody following incubation at 4° C. for six months in phosphate buffered saline.

In certain embodiments, an immunogen provided herein may be further modified to contain additional nonproteinaceous moities that are known in the art and readily available. The moities suitable for derivatization of the immunogen include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (cither homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the immunogen to be improved or altered, whether the immunogen derivative will be used in a therapy under defined conditions, etc.

Some of the sequences including recombinant NiV F or G ectodomain provided herein include the sequence of protease cleavage sites (such as thrombin sites), protein tags (such as a His tag, a Strep Tag II, a Avi tag, etc.), and signal peptides; such sequences can be removed from an isolated immunogen including a recombinant NiV F or G ectodomain trimer for therapeutic use.

### E. Protein Nanoparticles

In some embodiments, a protein nanoparticle is provided that includes one or more of the disclosed recombinant NiV F ectodomain trimers or a NiV G ectodomain. Non-limiting example of nanoparticles include ferritin nanoparticles, encapsulin nanoparticles, Sulfur Oxygenase Reductase (SOR) nanoparticles, and lumazine synthase nanoparticles, which are comprised of an assembly of monomeric subunits including ferritin proteins, encapsulin proteins, SOR proteins, and lumazine synthase, respectively. To construct such protein nanoparticles a protomer of the NiV F ectodomain trimer can be linked to a subunit of the protein nanoparticle (such as a ferritin protein, an encapsulin protein, a SOR protein, or a lumazine synthase protein) and expressed in cells under appropriate conditions. The fusion protein self-assembles into a nanoparticle any can be purified.

In some embodiments, a protomer of a disclosed recombinant NiV F ectodomain trimer, or a NiV G ectodomain, can be linked to a ferritin subunit to construct a ferritin nanoparticle. Ferritin nanoparticles and their use for immunization purposes (e.g., for immunization against influenza antigens) have been disclosed in the art (see, e.g., Kanekiyo et al., Nature, 499:102-106, 2013, incorporated by reference herein in its entirety). The globular form of the ferritin nanoparticle is made up of monomeric subunits, which are polypeptides having a molecule weight of approximately 17-20 kDa. Following production, these monomeric subunit proteins self-assemble into the globular ferritin protein. Thus, the globular form of ferritin comprises 24 monomeric, subunit proteins, and has a capsid-like structure having 432 symmetry. Methods of constructing ferritin nanoparticles are further described herein (see, e.g., Zhang, Int. J. Mol. Sci., 12:5406-5421, 2011, which is incorporated herein by reference in its entirety). An example of the amino acid sequence of one such monomeric ferritin subunit is represented by:

(SEQ ID NO: 55)
DIIKLLNEQVNKEMQSSNLYMSMSSWCYTHSLDGAGLFLFDHAAEEYEH
AKKLIIFLNENNVPVQLTSISAPEHKFEGLTQIFQKAYEHEQHISESIN
NIVDHAIKSKDHATFNFLQWYVAEQHEEEVLFKDILDKIELIGNENHGL
YLADQYVKGIAKSRKS

In specific examples, the ferritin polypeptide is E. coli ferritin, Helicobacter pylori ferritin, human light chain ferritin, bullfrog ferritin or a hybrid thereof, such as E. coli-human hybrid ferritin, E. coli-bullfrog hybrid ferritin, or human-bullfrog hybrid ferritin. Exemplary amino acid sequences of ferritin polypeptides and nucleic acid sequences encoding ferritin polypeptides for use to make a ferritin nanoparticle including a recombinant NiV F ectodomain trimer can be found in GENBANK®, for example at accession numbers ZP_03085328, ZP_06990637, EJB64322.1, AAA35832, NP_000137 AAA49532, AAA49525, AAA49524 and AAA49523, which are specifically incorporated by reference herein in their entirety as available Apr. 10, 2015. In some embodiments, a protomer of a recombinant NiV F ectodomain trimer can be linked to a ferritin subunit including an amino acid sequence at least 80% (such as at least 85%, at least 90%, at least 95%, or at least 97%) identical to amino acid sequence set forth as SEQ ID NO: 55.

In some embodiments, the self-assembling fusion proteins that form the ferritin nanoparticle comprise or consist of an amino acid sequence set forth as any one of residues 57-652 of SEQ ID NO: 38, residues 57-661 of SEQ ID NO: 39, residues 57-671 of SEQ ID NO: 40, residues 57-681 of SEQ ID NO: 41, or a sequence at least 90% identical to any one of residues 57-652 of SEQ ID NO: 38, residues 57-661 of SEQ ID NO: 39, residues 57-671 of SEQ ID NO: 40, residues 57-681 of SEQ ID NO: 41.

In some embodiments, a protomer of a disclosed recombinant NiV F ectodomain trimer, or a NiV G ectodomain, can be linked to a lumazine synthase subunit to construct a lumazine synthase nanoparticle. The globular form of lumazine synthase nanoparticle is made up of monomeric subunits; an example of the sequence of one such lumazine synthase subunit is provides as the amino acid sequence set forth as:

(SEQ ID NO: 56)
MQIYEGKLTAEGLRFGIVASRFNHALVDRLVEGAIDCIVRHGGREEDITL
VRVPGSWEIPVAAGELARKEDIDAVIAIGVLIRGATPHFDYIASEVSKGL
ADLSLELRKPITFGVITADTLEQAIERAGTKHGNKGWEAALSAIEMANLF
KSLR.

In some embodiments, a protomer of a disclosed recombinant NiV F ectodomain trimer, or a NiV G ectodomain, can be linked to a lumazine synthase subunit including an amino acid sequence at least 80% (such as at least 85%, at least 90%, at least 95%, or at least 97%) identical to amino acid sequence set forth as SEQ ID NO: 56.

In some embodiments, the self-assembling fusion proteins that form the lumazine synthase nanoparticle comprise or consist of an amino acid sequence set forth as any one of residues 57-647 of SEQ ID NO: 42, or a sequence at least 90% identical to any one of residues 57-647 of SEQ ID NO: 42.

In some embodiments, a protomer of a disclosed recombinant NiV F ectodomain trimer, or a NiV G ectodomain, can be linked to an encapsulin nanoparticle subunit to construct an encapsulin nanoparticle. The globular form of the encapsulin nanoparticle is made up of monomeric subunits; an example of the sequence of one such encapsulin subunit is provides as the amino acid sequence set forth as

(SEQ ID NO: 57)
MEFLKRSFAPLTEKQWQEIDNRAREIFKTQLYGRKFVDVEGPYGWEYAAH
PLGEVEVLSDENEVVKWGLRKSLPLIELRATFTLDLWELDNLERGKPNVD
LSSLEETVRKVAEFEDEVIFRGCEKSGVKGLLSFEERKIECGSTPKDLLE
AIVRALSIFSKDGIEGPYTLVINTDRWINFLKEEAGHYPLEKRVEECLRG
GKIITTPRIEDALVVSERGGDFKLILGQDLSIGYEDREKDAVRLFITETF
TFQVVNPEALILLKF.

In some embodiments, a protomer of a disclosed recombinant NiV F ectodomain trimer, or a NiV G ectodomain, can be linked to an encapsulin subunit including an amino acid sequence at least 80% (such as at least 85%, at least 90%, at least 95%, or at least 97%) identical to amino acid sequence set forth as SEQ ID NO: 57.

Encapsulin proteins are a conserved family of bacterial proteins also known as linocin-like proteins that form large protein assemblies that function as a minimal compartment to package enzymes. The encapsulin assembly is made up of monomeric subunits, which are polypeptides having a molecule weight of approximately 30 kDa. Following production, the monomeric subunits self-assemble into the globular encapsulin assembly including 60, or in some cases, 180 monomeric subunits. Methods of constructing encapsulin nanoparticles are further described (see, for example, Sutter et al., Nature Struct. and Mol. Biol., 15:939-947, 2008, which is incorporated by reference herein in its entirety). In specific examples, the encapsulin polypeptide is bacterial encapsulin, such as Thermotoga maritime or Pyrococcus furiosus or Rhodococcus erythropolis or Myxococcus xanthus encapsulin.

In some embodiments, a protomer of a disclosed recombinant NiV F ectodomain trimer, or a NiV G ectodomain, can be linked to a Sulfur Oxygenase Reductase (SOR) subunit to construct a recombinant SOR nanoparticle. In some embodiments, the SOR subunit can include the amino acid sequence set forth as

(SEQ ID NO: 58)
MEFLKRSFAPLTEKQWQEIDNRAREIFKTQLYGRKFVDVEGPYGWEYAAH
PLGEVEVLSDENEVVKWGLRKSLPLIELRATFTLDLWELDNLERGKPNVD
LSSLEETVRKVAEFEDEVIFRGCEKSGVKGLLSFEERKIECGSTPKDLLE
AIVRALSIFSKDGIEGPYTLVINTDRWINFLKEEAGHYPLEKRVEECLRG
GKIITTPRIEDALVVSERGGDFKLILGQDLSIGYEDREKDAVRLFITETF
TFQVVNPEALILLKF.

In some embodiments, a protomer of a disclosed recombinant NiV F ectodomain trimer, or a NiV G ectodomain, can be linked to a SOR subunit including an amino acid sequence at least 80% (such as at least 85%, at least 90%, at least 95%, or at least 97%) identical to amino acid sequence set forth as SEQ ID NO: 58.

SOR proteins are microbial proteins (for example from the thermoacidophilic archaeon Acidianus ambivalens that form 24 subunit protein assemblies. Methods of constructing SOR nanoparticles are described in Urich et al., Science, 311:996-1000, 2006, which is incorporated by reference herein in its entirety. An example of an amino acid sequence of a SOR protein for use to make SOR nanoparticles is set forth in Urich et al., Science, 311:996-1000, 2006, which is incorporated by reference herein in its entirety.

For production purposes, the recombinant NiV F ectodomain, or the NiV G ectodomain, linked to the nanoparticle subunit can include an N-terminal signal peptide that is cleaved during cellular processing. For example, the recombinant NiV F ectodomain protomer, or the NiV G ectodomain, linked to the protein nanoparticle subunit can include a signal peptide at its N-terminus including, for example, a native NiV F or G signal peptide.

The protein nanoparticles can be expressed in appropriate cells (e.g., HEK 293 Freestyle cells) and fusion proteins are secreted from the cells self-assembled into nanoparticles. The nanoparticles can be purified using known techniques, for example by a few different chromatography procedures, e.g. Mono Q (anion exchange) followed by size exclusion (SUPEROSE® 6) chromatography.

Several embodiments include a monomeric subunit of a ferritin, encapsulin, SOR, or lumazine synthase protein, or any portion thereof which is capable of directing self-assembly of monomeric subunits into the globular form of the protein. Amino acid sequences from monomeric subunits of any known ferritin, encapsulin, SOR, or lumazine synthase protein can be used to produce fusion proteins with the recombinant NiV F ectodomain, or the NiV G ectodomain, as long as the monomeric subunit is capable of self-assembling into a nanoparticle displaying the recombinant NiV F ectodomain trimer or the NiV G ectodomain on its surface.

The fusion proteins need not comprise the full-length sequence of a monomeric subunit polypeptide of a ferritin, encapsulin, SOR, or lumazine synthase protein. Portions, or regions, of the monomeric subunit polypeptide can be utilized so long as the portion comprises amino acid sequences that direct self-assembly of monomeric subunits into the globular form of the protein.

### II. Polynucleotides and Expression

Also provided are polynucleotides encoding any of the disclosed immunogens. For example, a polynucleotide encoding a protomer of a NiV F ectodomain trimer stabilized in the prefusion conformation, a chimera of the recombinant NiV F ectodomain trimer and one or more G ectodomains, a multimer of NiV G ectodomains, or a subunit of a self-assembling protein nanoparticle containing a recombinant NiV F or G ectodomain. These polynucleotides include DNA, cDNA and RNA sequences which encode the protomer. The genetic code can be used to construct a variety of functionally equivalent nucleic acids, such as nucleic acids which differ in sequence but which encode the same protein sequence, or encode a conjugate or fusion protein including the nucleic acid sequence.

In several embodiments, the nucleic acid molecule encodes a precursor of a protomer of the NiV F ectodomain trimer, that, when expressed in an appropriate cell, is processed into a protomer of the F ectodomain trimer that can self-assemble into the corresponding trimer. For example, the nucleic acid molecule can encode a protomer of the NiV F ectodomain trimer including a N-terminal signal sequence for entry into the cellular secretory system that is proteolytically cleaved in the during processing of the recombinant F ectodomain in the cell.

In some embodiments, the nucleic acid molecule encodes a F₀ polypeptide that, when expressed in an appropriate cell, is processed into a protomer of the NiV F ectodomain trimer including an F₂ polypeptide linked to a F₁ ectodomain, wherein the recombinant F₂-F₁ ectodomain protomer includes any of the prefusion-stabilizing modifications described herein, and optionally can be linked to a trimerization domain, such as a GCN4 trimerization domain.

In some embodiments, the nucleic acid molecule encodes a full-length F₀ polypeptide that, when expressed in an appropriate cell, is processed into a protomer of NiV F trimer including an F₂ polypeptide linked to a F₁ polypeptide including the F₁ TM and CT, wherein the recombinant F₂-F₁ ectodomain protomer includes any of the prefusion-stabilizing modifications described herein.

Exemplary nucleic acid sequences include:

NiVop08-TD (GCN4-Fd)-G
(SEQ ID NO: 61, which encodes SEQ ID NO: 43)
tctagagccaccatgtactctatgcagctggccagctgcgtgaccctgac
actggtgctgctggtgaactctcagggcatcctgcactacgagaagctga
gcaagatcggcctggtgaagggcgtgaccagaaagtataagatcaagtcc
aacccactgacaaaggacatcgtgatcaagatgatccccaacgtgagcaa
tatgtctcagtgtaccggctctgtgatggagaactacaagacccgcctga
atggcatcctgacaccaatcaagggcgccctggagatctataagaacaat
acacacgactgcgtgggcgatgtatgaggccatgaagaacgccgacaaca
tcaataagctgaagagctccatcgagagcaccaatgaggccgtggtgaag
ctgcaggagacagccgagaagacagtgtacgtgttcacagccctgcagga
ctatatcaacaccaatctggtgcccacaatcgataagatcccctgcaagc
agaccgagctgtccctggacctggccctgtctaagtacctgagcgatctg
ctgttcgtgtttggcccaaacctgcaggaccccgtgtccaattctatgac
aatccaggccatctcccaggccttcggcggcaattacgagacactgctga
gaacactgggctatgccaccgaggactttgacgatctgctggagagcgat
tccatcacaggccagatcatctatgtggatctgtctagctactatatcat
cgtgagggtgtacttccctatcctgaccgagatccagcaggcctatatcc
aggagctgctgccagtgagcttcaacaatgacaattccgagtggatctct
atcgtgcccaactttatcctggtgcggaacaccctgatcagcaatatcga
gatcggcttttgcctgatcacaaagagatccgtgatctgtaatcaggact
acgccacccccatgacaaacaatatgagggagtgcctgaccggctccaca
gagaagtgtccccgggagctggtggtgtcctctcacgtgcctagattcgc
cctgtccaacggcgtgctgtttgccaattgcatctctgtgacctgccagt
gtcagaccacaggaagggcaatctctcagagcggagagcagaccctgctg
atgatcgataacaccacatgtcctacagccgtgctgggcaatgtgatcat
cagcctgggcaagtacctgggctccgtgaactataattctgagggaatcg
caatcggaccacccgtgttcaccgacaaggtggatatcagctcccagatc
tctagcatgaaccagagcctgcagcagtccaaggactacatcaaggaggc
ccagcggctgctggataccgtgaatccttctctgaagctgatgaagcaga
tcgaggataagatcgaggagatcctgagcaagatctatcacatcgagaac
gagatcgccaggatcaagaagctgatcggagaggcacctggatctggtta
catcccagaggctccgcgggatggacaggcctacgtgagaaaggacggcg
agtgggtgctgctgagcaccttcctgggaagcggtggaggaggcggaggc
gtgagcaatctggtgggcctgcccaacaatatctgtctgcagaagacctc
caaccagatcctgaagcccaagctgatctcctatacactgcctgtggtgg
gccagtctggcacctgcatcacagaccctctgctggccatggatgagggc
tacttcgcctattctcacctggagaggatcggctcctgttctcgcggcgt
gagcaagcagcggatcatcggagtgggagaggtgctggacaggggcgatg
aggtgccttccctgttcatgaccaacgtgtggacaccacccaatccaaac
accgtgtaccactgctctgccgtgtataacaatgagttttactacgtgct
gtgcgccgtgagcaccgtgggcgatcctatcctgaactccacatactgga
gcggctccctgatgatgaccagactggccgtgaagccaaagtccaatggc
ggcggctataaccagcaccagctggccctgagatctatcgagaagggcag
gtacgataaagtgatgccttatggcccatctggcatcaagcagggcgaca
cactgtacttccccgccgtgggctttctggtgaggaccgagttcaagtac
aatgactccaactgccctatcacaaagtgtcagtattctaagccagagaa
ttgccgcctgagcatgggcatccggcccaactctcactacatcctgagaa
gcggcctgctgaagtataatctgagcgacggcgagaaccctaaggtggtg
tttatcgagatctccgatcagaggctgtctatcggctctcccagcaagat
ctacgactccctgggccagcccgtgttctaccaggcctccttttcttggg
acacaatgatcaagttcggcgatgtgctgaccgtgaatccactggtggtg
aactggagaaacaataccgtgatcagcaggccaggacagtcccagtgtcc
tcgctttaacacatgcccagagatctgttgggagggcgtgtacaatgacg
ccttcctgatcgatcggatcaactggatctccgccggcgtgtttctggac
tctaatcagaccgccgagaaccccgtgttcacagtgtttaaggataatga
gatcctgtacagggcacagctggcaagcgaggacaccaacgcccagaaga
ccatcacaaattgcttcctgctgaagaacaagatctggtgtatctccctg
gtggagatctatgacaccggcgataacgtgatccggccaaagctgtttgc
cgtgaagatccccgagcagtgcacaggcggcctggtgcctagaggctctc
accaccaccatcaccacagcgcctggtcccacccccagttcgagaagtga
taggatcc

G-NiVop08-TD (GCN4-Fd)
(SEQ ID NO: 62, which encodes SEQ ID NO: 44)
tctagaccaccatgtactcaatgcagctggcctcttgcgtcacactgaca
ctggtcctgctggtcaactcacagagaccacagaccgagggcgtgagcaa
tctggtgggcctgcccaacaatatctgtctgcagaagacctccaaccaga
tcctgaagcccaagctgatctcctatacactgcctgtggtgggccagtct
ggcacctgcatcacagaccctctgctggccatggatgagggctacttcgc
ctattctcacctggagaggatcggctcctgttctcgcggcgtgagcaagc
agcggatcatcggagtgggagaggtgctggacaggggcgatgaggtgcct
tccctgttcatgaccaacgtgtggacaccacccaatccaaacaccgtgta
ccactgctctgccgtgtataacaatgagttttactacgtgctgtgcgccg
tgagcaccgtgggcgatcctatcctgaactccacatactggagcggctcc
ctgatgatgaccagactggccgtgaagccaaagtccaatggcggcggcta
taaccagcaccagctggccctgagatctatcgagaagggcaggtacgata
aagtgatgccttatggcccatctggcatcaagcagggcgacacactgtac
ttccccgccgtgggctttctggtgaggaccgagttcaagtacaatgactc
caactgccctatcacaaagtgtcagtattctaagccagagaattgccgcc
tgagcatgggcatccggcccaactctcactacatcctgagaagcggcctg
ctgaagtataatctgagcgacggcgagaaccctaaggtggtgtttatcga
gatctccgatcagaggctgtctatcggctctcccagcaagatctacgact
ccctgggccagcccgtgttctaccaggcctccttttcttgggacacaatg
atcaagttcggcgatgtgctgaccgtgaatccactggtggtgaactggag
aaacaataccgtgatcagcaggccaggacagtcccagtgtcctcgcttta
acacatgcccagagatctgttgggagggcgtgtacaatgacgccttcctg
atcgatcggatcaactggatctccgccggcgtgtttctggactctaatca
gaccgccgagaaccccgtgttcacagtgtttaaggataatgagatcctgt
acagggcacagctggcaagcgaggacaccaacgcccagaagaccatcaca
aattgcttcctgctgaagaacaagatctggtgtatctccctggtggagat
ctatgacaccggcgataacgtgatccggccaaagctgtttgccgtgaaga
tccccgagcagtgcacaggcggcggtcagggcatcctgcactacgagaag
ctgagcaagatcggcctggtgaagggcgtgaccagaaagtataagatcaa
gtccaacccactgacaaaggacatcgtgatcaagatgatccccaacgtga
gcaatatgtctcagtgtaccggctctgtgatggagaactacaagacccgc
ctgaatggcatcctgacaccaatcaagggcgccctggagatctataagaa
caatacacacgactgcgtgggcgatgtgcggctggcaggcgtgtgcatgg
caggagtggcaatcggaatcgcaaccgcagcacagatcacagcaggagtg
gccctgtatgaggccatgaagaacgccgacaacatcaataagctgaagag
ctccatcgagagcaccaatgaggccgtggtgaagctgcaggagacagccg
agaagacagtgtacgtgttcacagccctgcaggactatatcaacaccaat
ctggtgcccacaatcgataagatcccctgcaagcagaccgagctgtccct
ggacctggccctgtctaagtacctgagcgatctgctgttcgtgtttggcc
caaacctgcaggaccccgtgtccaattctatgacaatccaggccatctcc
caggccttcggcggcaattacgagacactgctgagaacactgggctatgc
caccgaggactttgacgatctgctggagagcgattccatcacaggccaga
tcatctatgtggatctgtctagctactatatcatcgtgagggtgtacttc
cctatcctgaccgagatccagcaggcctatatccaggagctgctgccagt
gagcttcaacaatgacaattccgagtggatctctatcgtgcccaacttta
tcctggtgcggaacaccctgatcagcaatatcgagatcggcttttgcctg
atcacaaagagatccgtgatctgtaatcaggactacgccacccccatgac
aaacaatatgagggagtgcctgaccggctccacagagaagtgtccccggg
agctggtggtgtcctctcacgtgcctagattcgccctgtccaacggcgtg
ctgtttgccaattgcatctctgtgacctgccagtgtcagaccacaggaag
ggcaatctctcagagcggagagcagaccctgctgatgatcgataacacca
catgtcctacagccgtgctgggcaatgtgatcatcagcctgggcaagtac
ctgggctccgtgaactataattctgagggaatcgcaatcggaccacccgt
gttcaccgacaaggtggatatcagctcccagatctctagcatgaaccaga
gcctgcagcagtccaaggactacatcaaggaggcccagcggctgctggat
accgtgaatccttctctgaagctgatgaagcagatcgaggataagatcga
ggagatcctgagcaagatctatcacatcgagaacgagatcgccaggatca
agaagctgatcggagaggcacctggatctggttacatcccagaggctccg
cgggatggacaggcctacgtgagaaaggacggcgagtgggtgctgctgag
caccttcctgggaagcctggtgcctaggggctcccaccaccaccaccacc
acagcgcctggtcccacccacagtttgagaagtgatgaggatcc

NiV08
(SEQ ID NO: 63, which encodes SEQ ID NO: 24)
tctagagccaccatgtactctatgcagctggccagctgcgtgaccctgac
actggtgctgctggtgaactctcagggcatcctgcactacgagaagctga
gcaagatcggcctggtgaagggcgtgaccagaaagtataagatcaagtcc
aacccactgacaaaggacatcgtgatcaagatgatccccaacgtgagcaa
tatgtctcagtgtaccggctctgtgatggagaactacaagacccgcctga
atggcatcctgacaccaatcaagggcgccctggagatctataagaacaat
acacacgactgcgtgggcgatgtgcggctggcaggcgtgtgcatggcagg
agtggcaatcggaatcgcaaccgcagcacagatcacagcaggagtggccc
tgtatgaggccatgaagaacgccgacaacatcaataagctgaagagctcc
atcgagagcaccaatgaggccgtggtgaagctgcaggagacagccgagaa
gacagtgtacgtgttcacagccctgcaggactatatcaacaccaatctgg
tgcccacaatcgataagatcccctgcaagcagaccgagctgtccctggac
ctggccctgtctaagtacctgagcgatctgctgttcgtgtttggcccaaa
cctgcaggaccccgtgtccaattctatgacaatccaggccatctcccagg
ccttcggcggcaattacgagacactgctgagaacactgggctatgccacc
gaggactttgacgatctgctggagagcgattccatcacaggccagatcat
ctatgtggatctgtctagctactatatcatcgtgagggtgtacttcccta
tcctgaccgagatccagcaggcctatatccaggagctgctgccagtgagc
ttcaacaatgacgattccgagtggatctctatcgtgcccaactttatcct
ggtgcggaacaccctgatcagcaatatcgagatcggcttttgcctgatca
caaagagatccgtgatctgtaatcaggactacgccacccccatgacaaac
aatatgagggagtgcctgaccggctccacagagaagtgtccccgggagct
ggtggtgtcctctcacgtgcctagattcgccctgtccaacggcgtgctgt
ttgccaattgcatctctgtgacctgccagtgtcagaccacaggaagggca
atctctcagagcggagagcagaccctgctgatgatcgataacaccacatg
tcctacagccgtgctgggcaatgtgatcatcagcctgggcaagtacctgg
gctccgtgaactataattctgagggaatcgcaatcggaccacccgtgttc
accgacaaggtggatatcagctcccagatctctagcatgaaccagagcct
gcagcagtccaaggactacatcaaggaggcccagcggctgctggataccg
tgaatccttctctgaagctgatgaagcagatcgaggataagatcgaggag
atcctgagcaagatctatcacatcgagaacgagatcgccaggatcaagaa
gctgatcggagaggcacctggaggcctggtgccaaggggctcccaccacc
accaccaccacagcgcctggtcccacccacagtttgagaagtgatgagga
tcc

G-ln5-Fer
(SEQ ID NO: 64, which encodes SEQ ID NO: 38)
tctagaccaccatgtactcaatgcagctggcctcttgcgtcacactgaca
ctggtcctgctggtcaactcacagcaccaccaccatcatcacggaagcgc
ctggtcccaccctcagttcgagaagggaggactggtgcccagaggatccg
gcaacagccagcgccctcagaccgagggcgtgagcaatctggtgggcctg
cccaacaatatctgtctgcagaagacctccaaccagatcctgaagcccaa
gctgatctcctatacactgcctgtggtgggccagtctggcacctgcatca
cagaccctctgctggccatggatgagggctacttcgcctattctcacctg
gagaggatcggctcctgttctcgcggcgtgagcaagcagcggatcatcgg
agtgggagaggtgctggacaggggcgatgaggtgccttccctgttcatga
ccaacgtgtggacaccacccaatccaaacaccgtgtaccactgctctgcc
gtgtataacaatgagttttactacgtgctgtgcgccgtgagcaccgtggg
cgatcctatcctgaactccacatactggagcggctccctgatgatgacca
gactggccgtgaagccaaagtccaatggcggcggctataaccagcaccag
ctggccctgagatctatcgagaagggcaggtacgataaagtgatgcctta
tggcccatctggcatcaagcagggcgacacactgtacttccccgccgtgg
gctttctggtgaggaccgagttcaagtacaatgactccaactgccctatc
acaaagtgtcagtattctaagccagagaattgccgcctgagcatgggcat
ccggcccaactctcactacatcctgagaagcggcctgctgaagtataatc
tgagcgacggcgagaaccctaaggtggtgtttatcgagatctccgatcag
aggctgtctatcggctctcccagcaagatctacgactccctgggccagcc
cgtgttctaccaggcctccttttcttgggacacaatgatcaagttcggcg
atgtgctgaccgtgaatccactggtggtgaactggagaaacaataccgtg
atcagcaggccaggacagtcccagtgtcctcgctttaacacatgcccaga
gatctgttgggagggcgtgtacaatgacgccttcctgatcgatcggatca
actggatctccgccggcgtgtttctggactctaatcagaccgccgagaac
cccgtgttcacagtgtttaaggataatgagatcctgtacagggcacagct
ggcaagcgaggacaccaacgcccagaagaccatcacaaattgcttcctgc
tgaagaacaagatctggtgtatctccctggtggagatctatgacaccggc
gataacgtgatccggccaaagctgtttgccgtgaagatccccgagcagtg
cacaggcggcggcagcggcggggatatcattaagctgctgaacgaacagg
tgaacaaggagatgcagtcaagcaacctgtacatgtctatgtcctcttgg
tgctatacacatagtctggacggagctggcctgttcctgtttgatcacgc
agccgaggaatacgaacatgcaaagaaactgatcattttcctgaatgaga
acaatgtgccagtccagctgacaagtatctcagcccccgaacacaagttc
gaggggctgactcagatctttcagaaagcttacgaacacgagcagcatat
tagcgaatccatcaacaatattgtggaccacgctatcaagtccaaagatc
atgcaaccttcaactttctgcagtggtacgtggccgagcagcacgaggaa
gaggtcctgtttaaggacatcctggataaaatcgaactgattggcaacga
gaatcatgggctgtacctggccgatcagtatgtgaagggcattgctaagt
cacggaaaagcggaagctgatgaccgcgg

NiVop08-GCN4-G
(SEQ ID NO: 65, which encodes SEQ ID NO: 59)
tctagagccaccatgtactctatgcagctggccagctgcgtgaccctgac
actggtgctgctggtgaactctcagggcatcctgcactacgagaagctga
gcaagatcggcctggtgaagggcgtgaccagaaagtataagatcaagtcc
aacccactgacaaaggacatcgtgatcaagatgatccccaacgtgagcaa
tatgtctcagtgtaccggctctgtgatggagaactacaagacccgcctga
atggcatcctgacaccaatcaagggcgccctggagatctataagaacaat
acacacgactgcgtgggcgatgtgcggctggcaggcgtgtgcatggcagg
agtggcaatcggaatcgcaaccgcagcacagatcacagcaggagtggccc
tgtatgaggccatgaagaacgccgacaacatcaataagctgaagagctcc
atcgagagcaccaatgaggccgtggtgaagctgcaggagacagccgagaa
gacagtgtacgtgttcacagccctgcaggactatatcaacaccaatctgg
tgcccacaatcgataagatcccctgcaagcagaccgagctgtccctggac
ctggccctgtctaagtacctgagcgatctgctgttcgtgtttggcccaaa
cctgcaggaccccgtgtccaattctatgacaatccaggccatctcccagg
ccttcggcggcaattacgagacactgctgagaacactgggctatgccacc
gaggactttgacgatctgctggagagcgattccatcacaggccagatcat
ctatgtggatctgtctagctactatatcatcgtgagggtgtacttcccta
tcctgaccgagatccagcaggcctatatccaggagctgctgccagtgagc
ttcaacaatgacaattccgagtggatctctatcgtgcccaactttatcct
ggtgcggaacaccctgatcagcaatatcgagatcggcttttgcctgatca
caaagagatccgtgatctgtaatcaggactacgccacccccatgacaaac
aatatgagggagtgcctgaccggctccacagagaagtgtccccgggagct
ggtggtgtcctctcacgtgcctagattcgccctgtccaacggcgtgctgt
ttgccaattgcatctctgtgacctgccagtgtcagaccacaggaagggca
atctctcagagcggagagcagaccctgctgatgatcgataacaccacatg
tcctacagccgtgctgggcaatgtgatcatcagcctgggcaagtacctgg
gctccgtgaactataattctgagggaatcgcaatcggaccacccgtgttc
accgacaaggtggatatcagctcccagatctctagcatgaaccagagcct
gcagcagtccaaggactacatcaaggaggcccagcggctgctggataccg
tgaatccttctctgaagctgatgaagcagatcgaggataagatcgaggag
atcctgagcaagatctatcacatcgagaacgagatcgccaggatcaagaa
gctgatcggagaggcacctggatctggtggaggaggcggaggcgtgagca
atctggtgggcctgcccaacaatatctgtctgcagaagacctccaaccag
atcctgaagcccaagctgatctcctatacactgcctgtggtgggccagtc
tggcacctgcatcacagaccctctgctggccatggatgagggctacttcg
cctattctcacctggagaggatcggctcctgttctcgcggcgtgagcaag
cagcggatcatcggagtgggagaggtgctggacaggggcgatgaggtgcc
ttccctgttcatgaccaacgtgtggacaccacccaatccaaacaccgtgt
accactgctctgccgtgtataacaatgagttttactacgtgctgtgcgcc
gtgagcaccgtgggcgatcctatcctgaactccacatactggagcggctc
cctgatgatgaccagactggccgtgaagccaaagtccaatggcggcggct
ataaccagcaccagctggccctgagatctatcgagaagggcaggtacgat
aaagtgatgccttatggcccatctggcatcaagcagggcgacacactgta
cttccccgccgtgggctttctggtgaggaccgagttcaagtacaatgact
ccaactgccctatcacaaagtgtcagtattctaagccagagaattgccgc
ctgagcatgggcatccggcccaactctcactacatcctgagaagcggcct
gctgaagtataatctgagcgacggcgagaaccctaaggtggtgtttatcg
agatctccgatcagaggctgtctatcggctctcccagcaagatctacgac
tccctgggccagcccgtgttctaccaggcctccttttcttgggacacaat
gatcaagttcggcgatgtgctgaccgtgaatccactggtggtgaactgga
gaaacaataccgtgatcagcaggccaggacagtcccagtgtcctcgcttt
aacacatgcccagagatctgttgggagggcgtgtacaatgacgccttcct
gatcgatcggatcaactggatctccgccggcgtgtttctggactctaatc
agaccgccgagaaccccgtgttcacagtgtttaaggataatgagatcctg
tacagggcacagctggcaagcgaggacaccaacgcccagaagaccatcac
aaattgcttcctgctgaagaacaagatctggtgtatctccctggtggaga
tctatgacaccggcgataacgtgatccggccaaagctgtttgccgtgaag
atccccgagcagtgcacaggcggcctggtgcctagaggctctcaccacca
ccatcaccacagcgcctggtcccacccccagttcgagaagtgataggatc
c

NiVop08-Fd-G
(SEQ ID NO: 66, which encodes SEQ ID NO: 60)
tctagagccaccatgtactctatgcagctggccagctgcgtgaccctgac
actggtgctgctggtgaactctcagggcatcctgcactacgagaagctga
gcaagatcggcctggtgaagggcgtgaccagaaagtataagatcaagtcc
aacccactgacaaaggacatcgtgatcaagatgatccccaacgtgagcaa
tatgtctcagtgtaccggctctgtgatggagaactacaagacccgcctga
atggcatcctgacaccaatcaagggcgccctggagatctataagaacaat
acacacgactgcgtgggcgatgtgcggctggcaggcgtgtgcatggcagg
agtggcaatcggaatcgcaaccgcagcacagatcacagcaggagtggccc
tgtatgaggccatgaagaacgccgacaacatcaataagctgaagagctcc
atcgagagcaccaatgaggccgtggtgaagctgcaggagacagccgagaa
gacagtgtacgtgttcacagccctgcaggactatatcaacaccaatctgg
tgcccacaatcgataagatcccctgcaagcagaccgagctgtccctggac
ctggccctgtctaagtacctgagcgatctgctgttcgtgtttggcccaaa
cctgcaggaccccgtgtccaattctatgacaatccaggccatctcccagg
ccttcggcggcaattacgagacactgctgagaacactgggctatgccacc
gaggactttgacgatctgctggagagcgattccatcacaggccagatcat
ctatgtggatctgtctagctactatatcatcgtgagggtgtacttcccta
tcctgaccgagatccagcaggcctatatccaggagctgctgccagtgagc
ttcaacaatgacaattccgagtggatctctatcgtgcccaactttatcct
ggtgcggaacaccctgatcagcaatatcgagatcggcttttgcctgatca
caaagagatccgtgatctgtaatcaggactacgccacccccatgacaaac
aatatgagggagtgcctgaccggctccacagagaagtgtccccgggagct
ggtggtgtcctctcacgtgcctagattcgccctgtccaacggcgtgctgt
ttgccaattgcatctctgtgacctgccagtgtcagaccacaggaagggca
atctctcagagcggagagcagaccctgctgatgatcgataacaccacatg
tcctacagccgtgctgggcaatgtgatcatcagcctgggcaagtacctgg
gctccgtgaactataattctgagggaatcgcaatcggaccacccgtgttc
accgacaaggtggatatcagctcccagatctctagcatgaaccagagcct
gcagcagtccaaggactacatcaaggaggcccagcggctgctggataccg
tgaatccttctctgggatctggttacatcccagaggctccgcgggatgga
caggcctacgtgagaaaggacggcgagtgggtgctgctgagcaccttcct
gggaagcggtggaggaggcggaggcgtgagcaatctggtgggcctgccca
acaatatctgtctgcagaagacctccaaccagatcctgaagcccaagctg
atctcctatacactgcctgtggtgggccagtctggcacctgcatcacaga
ccctctgctggccatggatgagggctacttcgcctattctcacctggaga
ggatcggctcctgttctcgcggcgtgagcaagcagcggatcatcggagtg
ggagaggtgctggacaggggcgatgaggtgccttccctgttcatgaccaa
cgtgtggacaccacccaatccaaacaccgtgtaccactgctctgccgtgt
ataacaatgagttttactacgtgctgtgcgccgtgagcaccgtgggcgat
cctatcctgaactccacatactggagcggctccctgatgatgaccagact
ggccgtgaagccaaagtccaatggcggcggctataaccagcaccagctgg
ccctgagatctatcgagaagggcaggtacgataaagtgatgccttatggc
ccatctggcatcaagcagggcgacacactgtacttccccgccgtgggctt
tctggtgaggaccgagttcaagtacaatgactccaactgccctatcacaa
agtgtcagtattctaagccagagaattgccgcctgagcatgggcatccgg
cccaactctcactacatcctgagaagcggcctgctgaagtataatctgag
cgacggcgagaaccctaaggtggtgtttatcgagatctccgatcagaggc
tgtctatcggctctcccagcaagatctacgactccctgggccagcccgtg
ttctaccaggcctccttttcttgggacacaatgatcaagttcggcgatgt
gctgaccgtgaatccactggtggtgaactggagaaacaataccgtgatca
gcaggccaggacagtcccagtgtcctcgctttaacacatgcccagagatc
tgttgggagggcgtgtacaatgacgccttcctgatcgatcggatcaactg
gatctccgccggcgtgtttctggactctaatcagaccgccgagaaccccg
tgttcacagtgtttaaggataatgagatcctgtacagggcacagctggca
agcgaggacaccaacgcccagaagaccatcacaaattgcttcctgctgaa
gaacaagatctggtgtatctccctggtggagatctatgacaccggcgata
acgtgatccggccaaagctgtttgccgtgaagatccccgagcagtgcaca
ggcggcctggtgcctagaggctctcaccaccaccatcaccacagcgcctg
gtcccacccccagttcgagaagtgataggatcc

G-Fd-G
(SEQ ID NO: 67, which encodes SEQ ID NO: 37)
tctagagccaccatgtactctatgcagctggccagctgcgtgaccctgac
actggtgctgctggtgaactctcaggagggcgtgagcaatctggtgggcc
tgcccaacaatatctgtctgcagaagacctccaaccagatcctgaagccc
aagctgatctcctatacactgcctgtggtgggccagtctggcacctgcat
cacagaccctctgctggccatggatgagggctacttcgcctattctcacc
tggagaggatcggctcctgttctcgcggcgtgagcaagcagcggatcatc
ggagtgggagaggtgctggacaggggcgatgaggtgccttccctgttcat
gaccaacgtgtggacaccacccaatccaaacaccgtgtaccactgctctg
ccgtgtataacaatgagttttactacgtgctgtgcgccgtgagcaccgtg
ggcgatcctatcctgaactccacatactggagcggctccctgatgatgac
cagactggccgtgaagccaaagtccaatggcggcggctataaccagcacc
agctggccctgagatctatcgagaagggcaggtacgataaagtgatgcct
tatggcccatctggcatcaagcagggcgacacactgtacttccccgccgt
gggctttctggtgaggaccgagttcaagtacaatgactccaactgcccta
tcacaaagtgtcagtattctaagccagagaattgccgcctgagcatgggc
atccggcccaactctcactacatcctgagaagcggcctgctgaagtataa
tctgagcgacggcgagaaccctaaggtggtgtttatcgagatctccgatc
agaggctgtctatcggctctcccagcaagatctacgactccctgggccag
cccgtgttctaccaggcctccttttcttgggacacaatgatcaagttcgg
cgatgtgctgaccgtgaatccactggtggtgaactggagaaacaataccg
tgatcagcaggccaggacagtcccagtgtcctcgctttaacacatgccca
gagatctgttgggagggcgtgtacaatgacgccttcctgatcgatcggat
caactggatctccgccggcgtgtttctggactctaatcagaccgccgaga
accccgtgttcacagtgtttaaggataatgagatcctgtacagggcacag
ctggcaagcgaggacaccaacgcccagaagaccatcacaaattgcttcct
gctgaagaacaagatctggtgtatctccctggtggagatctatgacaccg
gcgataacgtgatccggccaaagctgtttgccgtgaagatccccgagcag
tgcacaggcggcggatctggttacatcccagaggctccgcgggatggaca
ggcctacgtgagaaaggacggcgagtgggtgctgctgagcaccttcctgg
gaagcggtggaggaggcggaggcgtgagcaatctggtgggcctgcccaac
aatatctgtctgcagaagacctccaaccagatcctgaagcccaagctgat
ctcctatacactgcctgtggtgggccagtctggcacctgcatcacagacc
ctctgctggccatggatgagggctacttcgcctattctcacctggagagg
atcggctcctgttctcgcggcgtgagcaagcagcggatcatcggagtggg
agaggtgctggacaggggcgatgaggtgccttccctgttcatgaccaacg
tgtggacaccacccaatccaaacaccgtgtaccactgctctgccgtgtat
aacaatgagttttactacgtgctgtgcgccgtgagcaccgtgggcgatcc
tatcctgaactccacatactggagcggctccctgatgatgaccagactgg
ccgtgaagccaaagtccaatggcggcggctataaccagcaccagctggcc
ctgagatctatcgagaagggcaggtacgataaagtgatgccttatggccc
atctggcatcaagcagggcgacacactgtacttccccgccgtgggctttc
tggtgaggaccgagttcaatacaatgactccaactgccctatcacaaagt
gtcagtattctaagccagagaattgccgcctgagcatgggcatccggccc
aactctcactacatcctgagaagcggcctgctgaagtataatctgagcga
cggcgagaaccctaaggtggtgtttatcgagatctccgatcagaggctgt
ctatcggctctcccagcaagatctacgactccctgggccagcccgtgttc
taccaggcctccttttcttgggacacaatgatcaagttcggcgatgtgct
gaccgtgaatccactggtggtgaactggagaaacaataccgtgatcagca
ggccaggacagtcccagtgtcctcgctttaacacatgcccagagatctgt
tgggagggcgtgtacaatgacgccttcctgatcgatcggatcaactggat
ctccgccggcgtgtttctggactctaatcagaccgccgagaaccccgtgt
tcacagtgtttaaggataatgagatcctgtacagggcacagctggcaagc
gaggacaccaacgcccagaagaccatcacaaattgcttcctgctgaagaa
caagatctggtgtatctccctggtggagatctatgacaccggcgataacg
tgatccggccaaagctgtttgccgtgaagatccccgagcagtgcacaggc
ggcctggtgcctagaggctctcaccaccaccatcaccacagcgcctggtc
ccacccccagttcgagaagtgataggatcc

Full-length NiV F with NiVop08 substitutions
(SEQ ID NO: 33)
tctagagccaccatggtggtcatcctggacaagagatgctactgtaacct
gctgatcctgatcctgatgatcagcgagtgctccgtgggcatcctgcact
acgagaagctgtccaagatcggcctggtgaagggcgtgaccaggaagtat
aagatcaagtctaatcccctgacaaaggatatcgtgatcaagatgatccc
taacgtgtctaatatgagccagtgtaccggctccgtgatggagaactaca
agaccagactgaatggcatcctgacacccatcaagggcgccctggagatc
tataagaacaatacacacgactgcgtgggcgatgtgaggctggcaggcgt
gtgcatggcaggagtggcaatcggaatccaaccgcagcacagatcacagc
aggagtggccctgtatgaggccatgaagaacgccgacaacatcaataagc
tgaagagctccatcgagagcaccaatgaggccgtggtgaagctgcaggag
accgccgagaagacagtgtacgtgttcacagccctgcaggactatatcaa
caccaatctggtgcctacaatcgataagatcccttgcaagcagaccgagc
tgagcctggacctggccctgagcaagtacctgtccgatctgctgttcgtg
tttggcccaaacctgcaggaccccgtgagcaattccatgacaatccaggc
catctcccaggccttcggcggcaactacgagaccctgctgcgcacactgg
gctatgccaccgaggactttgacgatctgctggagtctgatagcatcaca
ggccagatcatctatgtggacctgtctagctactatatcatcgtgcgggt
gtacttcccaatcctgaccgagatccagcaggcctatatccaggagctgc
tgcccgtgtccttcaacaatgataactctgagtggatcagcatcgtgcct
aacttcatcctggtgcggaacaccctgatctctaatatcgagatcggctt
ttgcctgatcacaaagcgcagcgtgatctgtaatcaggactacgccaccc
ctatgacaaacaatatgcgggagtgcctgaccggcagcacagagaagtgt
cctcgggagctggtggtgtcctctcacgtgccaagattcgccctgtccaa
cggcgtgctgtttgccaattgcatctctgtgacctgccagtgtcagacca
caggcagggccatctcccagtctggcgagcagaccctgctgatgatcgat
aacaccacatgtccaacagccgtgctgggcaatgtgatcatcagcctggg
caagtacctgggcagcgtgaactataattccgagggaatcgcaatcggac
cacccgtgttcaccgacaaggtggatatcagctcccagatctctagcatg
aaccagtccctgcagcagtctaaggactacatcaaggaggcccagcgcct
gctggataccgtgaatccatccctgatctctatgctgagcatgatcatcc
tgtatgtgctgtccatcgcctctctgtgcatcggcctgatcaccttcatc
agctttatcatcgtggagaagaagaggaacacatactcccgcctggagga
caggagagtgcggcccacctcctctggcgatctgtactatatcggcacat
gatgaggatcc

Exemplary nucleic acids can be prepared by cloning techniques. Examples of appropriate cloning and sequencing techniques, and instructions sufficient to direct persons of skill through many cloning exercises are known (see. e.g., Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013).

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

The polynucleotides encoding a protomer of the NiV F ectodomain trimer can include a recombinant DNA which is incorporated into a vector (such as an expression vector) into an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single and double forms of DNA.

Polynucleotide sequences encoding a protomer of the NiV F ectodomain trimer can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

DNA sequences encoding the protomer of the NiV F ectodomain trimer can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Hosts can include microbial, yeast, insect and mammalian organisms. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, e.g., Helgason and Miller (Eds.), 2012, Basic Cell Culture Protocols (Methods in Molecular Biology), 4^{th} Ed., Humana Press). Examples of commonly used mammalian host cell lines are VERO and HeLa cells, CHO cells, and WI38, BHK, and COS cell lines, although cell lines may be used, such as cells designed to provide higher expression, desirable glycosylation patterns, or other features. In some embodiments, the host cells include HEK293 cells or derivatives thereof, such as GnTI^{-/-} cells (ATCC® No. CRL-3022), or HEK-293F cells.

Transformation of a host cell with recombinant DNA can be carried out by conventional techniques. In some embodiments where the host is prokaryotic, such as, but not limited to, E. coli, competent cells which are capable of DNA uptake can be prepared from cells harvested after exponential growth phase and subsequently treated by the CaCl₂) method. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or viral vectors can be used. Eukaryotic cells can also be co-transformed with polynucleotide sequences encoding a disclosed antigen, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Viral Expression Vectors, Springer press, Muzyczka cd., 2011). Appropriate expression systems such as plasmids and vectors of use in producing proteins in cells including higher eukaryotic cells such as the COS, CHO, HeLa and myeloma cell lines.

In one non-limiting example, a disclosed immunogen is expressed using the pVRC8400 vector (described in Barouch et al., J. Virol., 79, 8828-8834, 2005, which is incorporated by reference herein).

Modifications can be made to a nucleic acid encoding a disclosed immunogen without diminishing its biological activity. Some modifications can be made to facilitate the cloning, expression, or incorporation of the targeting molecule into a fusion protein. Exemplary modifications include termination codons, a methionine added at the amino terminus to provide an initiation, site, additional amino acids placed on either terminus to create conveniently located restriction sites, or additional amino acids (such as poly His) to aid in purification steps.

In some embodiments, the nucleic acid encoding the protomer of a disclosed recombinant NiV F ectodomain protomer can be expressed in cells under conditions where the protomers self-assemble into trimers which are secreted from the cells into the cell media, for example as described for RSV F proteins (see, e.g., PCT Pub. WO2014160463, Mclellan et al., Science, 340:1113-1117, 2013; Mclellan et al., Science, 342:592-598, 2013, each of which is incorporated by reference herein in its entirety). In such embodiments, the protomer contains a leader sequence (signal peptide) that causes the protein to enter the secretory system, and the signal peptide is cleaved and the protomers form a trimer, before being secreted in the cell media. The medium can be centrifuged and recombinant NiV F ectodomain trimer purified from the supernatant.

### III. Viral Vectors

A nucleic acid molecule encoding a disclosed immunogen can be included in a viral vector, for example, for expression of the immunogen in a host cell, or for immunization of a subject as disclosed herein. In some embodiments, the viral vectors are administered to a subject as part of a prime-boost vaccination. Typically such viral vectors include a nucleic acid molecule encoding an immunogen that contains a transmembrane domain. In several embodiments, the viral vectors are included in a vaccine, such as a primer vaccine or a booster vaccine for use in a prime-boost vaccination.

In some examples, the viral vector can be replication-competent. For example, the viral vector can have a mutation (e.g., insertion of nucleic acid encoding the protomer) in the viral genome that attenuates, but does not completely block viral replication in host cells.

In several embodiments, the viral vector can be delivered via the respiratory tract. For example, a hPIV vector, such as bovine parainfluenza virus (BPIV) vector (e.g., a BPIV1, BPIV2, or BPIV3 vector) or human hPIV vector (e.g., a hPIV3 vector), a metapneumovirus (MPV) vector, a Sendia virus vector, or a measles virus vector, is used to express a disclosed antigen.

Additional viral vectors are also available for expression of the disclosed antigens, including polyoma, i.e., SV40 (Madzak et al., 1992, J. Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Natl. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV and CMV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

### IV. Virus-Like Particles

In some embodiments, a virus-like particle (VLP) is provided that includes a disclosed immunogen. Typically such VLPs include an immunogen containing a transmembrane domain, for example, a recombinant NiV F ectodomain trimer with protomers containing a NiV F transmembrane domain and cytosolic tail. VLPs lack the viral components that are required for virus replication and thus represent a highly attenuated, replication-incompetent form of a virus. However, the VLP can display a polypeptide (e.g., a recombinant NiV F ectodomain trimer) that is analogous to that expressed on infectious virus particles and can eliciting an immune response to NiV when administered to a subject. Exemplary virus like particles and methods of their production, as well as viral proteins from several viruses that are known to form VLPs, including human papillomavirus, HIV (Kang et al., Biol. Chem. 380: 353-64 (1999)), Semliki-Forest virus (Notka et al., Biol. Chem. 380: 341-52 (1999)), human polyomavirus (Goldmann et al., J. Virol. 73: 4465-9 (1999)), rotavirus (Jiang et al., Vaccine 17: 1005-13 (1999)), parvovirus (Casal, Biotechnology and Applied Biochemistry, Vol 29, Part 2, pp 141-150 (1999)), canine parvovirus (Hurtado et al., J. Virol. 70: 5422-9 (1996)), hepatitis E virus (Li et al., J. Virol. 71: 7207-13 (1997)), and Newcastle disease virus. The formation of such VLPs can be detected by any suitable technique. Examples of suitable techniques for detection of VLPs in a medium include, e.g., electron microscopy techniques, dynamic light scattering (DLS), selective chromatographic separation (e.g., ion exchange, hydrophobic interaction, and/or size exclusion chromatographic separation of the VLPs) and density gradient centrifugation.

### V. Immunogenic Compositions

Immunogenic compositions comprising a disclosed immunogen (e.g., recombinant NiV F ectodomain trimer, a nucleic acid molecule or vector encoding a protomer of the recombinant NiV F ectodomain trimer, or a protein nanoparticle or virus like particle comprising a disclosed recombinant NiV F ectodomain trimer) and a pharmaceutically acceptable carrier are also provided. Such compositions can be administered to subjects by a variety of administration modes, for example, intramuscular, subcutaneous, intravenous, intra-arterial, intra-articular, intraperitoneal, or parenteral routes. In several embodiments, a pharmaceutical composition including one or more of the disclosed immunogens are immunogenic compositions. Actual methods for preparing administrable compositions are described in more detail in such publications as Remingtons Pharmaceutical Sciences, 19th Ed., Mack Publishing Company, Easton, Pennsylvania, 1995.

Thus, an immunogen described herein can be formulated with pharmaceutically acceptable carriers to help retain biological activity while also promoting increased stability during storage within an acceptable temperature range. Potential carriers include, but are not limited to, physiologically balanced culture medium, phosphate buffer saline solution, water, emulsions (e.g., oil/water or water/oil emulsions), various types of wetting agents, cryoprotective additives or stabilizers such as proteins, peptides or hydrolysates (e.g., albumin, gelatin), sugars (e.g., sucrose, lactose, sorbitol), amino acids (e.g., sodium glutamate), or other protective agents. The resulting aqueous solutions may be packaged for use as is or lyophilized. Lyophilized preparations are combined with a sterile solution prior to administration for either single or multiple dosing.

Formulated compositions, especially liquid formulations, may contain a bacteriostat to prevent or minimize degradation during storage, including but not limited to effective concentrations (usually ≤1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The immunogenic compositions of the disclosure can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate.

The immunogenic composition may optionally include an adjuvant to enhance an immune response of the host. Adjuvants, such as aluminum hydroxide (ALHYDROGEL®, available from Brenntag Biosector, Copenhagen, Denmark and Amphogel®, Wyeth Laboratories, Madison, NJ), Freund's adjuvant, MPL™ (3-O-deacylated monophosphoryl lipid A; Corixa, Hamilton, IN) and IL-12 (Genetics Institute, Cambridge, MA), TLR agonists (such as TLR-9 agonists), among many other suitable adjuvants well known in the art, can be included in the compositions. Suitable adjuvants are, for example, toll-like receptor agonists, alum, AlPO4, alhydrogel, Lipid-A and derivatives or variants thereof, oil-emulsions, saponins, neutral liposomes, liposomes containing the vaccine and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, IN) and IL-12 (Genetics Institute, Cambridge, MA), may be used as an adjuvant (Newman et al., 1998, Critical Reviews in Therapeutic Drug Carrier Systems 15:89-142). These adjuvants have the advantage in that they help to stimulate the immune system in a non-specific way, thus enhancing the immune response to a pharmaceutical product.

In some instances, the adjuvant formulation is a mineral salt, such as a calcium or aluminum (alum) salt, for example calcium phosphate, aluminum phosphate or aluminum hydroxide. In some embodiments, the adjuvant includes an oil and water emulsion, e.g., an oil-in-water emulsion (such as MF59 (Novartis) or AS03 (GlaxoSmithKline). One example of an oil-in-water emulsion comprises a metabolisable oil, such as squalene, a tocol such as a tocopherol, e.g., alpha-tocopherol, and a surfactant, such as sorbitan trioleate (Span 85) or polyoxyethylene sorbitan monooleate (Tween 80), in an aqueous carrier.

In some instances it may be desirable to combine a disclosed immunogen with other pharmaceutical products (e.g., vaccines) which induce protective responses to other agents. For example, a composition including a recombinant NiV F ectodomain trimer as described herein can be can be administered simultaneously (typically separately) or sequentially with other vaccines recommended by the Advisory Committee on Immunization Practices (ACIP; cdc.gov/vaccines/acip/index.html) for the targeted age group (e.g., infants from approximately one to six months of age). As such, a disclosed immunogen described herein may be administered simultaneously or sequentially with vaccines against, for example, hepatitis B (HepB), diphtheria, tetanus and pertussis (DTaP), pneumococcal bacteria (PCV), Haemophilus influenzae type b (Hib), polio, influenza and rotavirus.

In some embodiments, the composition can be provided as a sterile composition. The immunogenic composition typically contains an effective amount of a disclosed immunogen and can be prepared by conventional techniques. Typically, the amount of immunogen in each dose of the immunogenic composition is selected as an amount which induces an immune response without significant, adverse side effects. In some embodiments, the composition can be provided in unit dosage form for use to induce an immune response in a subject, for example, to inhibit NiV infection in the subject. A unit dosage form contains a suitable single preselected dosage for administration to a subject, or suitable marked or measured multiples of two or more preselected unit dosages, and/or a metering mechanism for administering the unit dose or multiples thereof.

### VI. Methods of Inducing an Immune Response

The disclosed immunogens (e.g., recombinant prefusion-stabilized NiV F ectodomain trimer, a nucleic acid molecule (such as an RNA molecule) or vector encoding a protomer of the prefusion-stabilized NiV F ectodomain trimer, or a protein nanoparticle or virus like particle comprising the prefusion-stabilized NiV F ectodomain trimer) can be administered to a subject to induce an immune response to NiV in the subject. In a particular example, the subject is a human. The immune response can be a protective immune response, for example a response that inhibits subsequent infection with NiV. Elicitation of the immune response can also be used to treat or inhibit NiV infection and illnesses associated therewith.

A subject can be selected for treatment that has, or is at risk for developing NiV infection, for example because of exposure or the possibility of exposure to NiV. Following administration of a disclosed immunogen, the subject can be monitored for the NiV infection or symptoms associated therewith, or both.

Typical subjects intended for treatment with the therapeutics and methods of the present disclosure include humans and domestic animals such as pigs. In several embodiments, the subject is a human subject that is seronegative for NiV specific antibodies. To identify subjects for prophylaxis or treatment according to the methods of the disclosure, accepted screening methods are employed to determine risk factors associated with a targeted or suspected disease or condition, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods to detect and/or characterize NiV infection. These and other routine methods allow the clinician to select patients in need of therapy using the methods and immunogenic compositions of the disclosure. In accordance with these methods and principles, a composition can be administered according to the teachings herein, or other conventional methods, as an independent prophylaxis or treatment program, or as a follow-up, adjunct or coordinate treatment regimen to other treatments.

The administration of a disclosed immunogen can be for prophylactic or therapeutic purpose. When provided prophylactically, the immunogen can be provided in advance of any symptom, for example in advance of infection. The prophylactic administration serves to prevent or ameliorate any subsequent infection. In some embodiments, the methods can involve selecting a subject at risk for contracting NiV infection, and administering a therapeutically effective amount of a disclosed immunogen to the subject. The immunogen can be provided prior to the anticipated exposure to NiV so as to attenuate the anticipated severity, duration or extent of an infection and/or associated disease symptoms, after exposure or suspected exposure to the virus, or after the actual initiation of an infection. When provided therapeutically, the disclosed immunogens are provided at or after the onset of a symptom of NiV infection, or after diagnosis of NiV infection. Treatment of NiV by inhibiting NiV replication or infection can include delaying and/or reducing signs or symptoms of NiV infection in a subject. In some examples, treatment using the methods disclosed herein prolongs the time of survival of the subject.

In some embodiments, administration of a disclosed immunogen to a subject can elicit the production of an immune response that is protective against serious lower respiratory tract disease, such as pneumonia and bronchiolitis, or croup, when the subject is subsequently infected or re-infected with a wild-type NiV. While the naturally circulating virus may still be capable of causing infection, particularly in the upper respiratory tract, there can be a reduced possibility of rhinitis as a result of the vaccination and a possible boosting of resistance by subsequent infection by wild-type virus. Following vaccination, there are detectable levels of host engendered serum and secretory antibodies which are capable of neutralizing homologous (of the same subgroup) wild-type virus in vitro and in vivo. In many instances the host antibodies will also neutralize wild-type virus of a different, non-vaccine subgroup.

The immunogens described herein, and immunogenic compositions thereof, are provided to a subject in an amount effective to induce or enhance an immune response against NiV in the subject, preferably a human. The actual dosage of disclosed immunogen will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimum prophylactic or therapeutic response.

An immunogenic composition including one or more of the disclosed immunogens can be used in coordinate (or prime-boost) vaccination protocols or combinatorial formulations. In certain embodiments, novel combinatorial immunogenic compositions and coordinate immunization protocols employ separate immunogens or formulations, each directed toward eliciting an anti-viral immune response, such as an immune response to NiV F protein. Separate immunogenic compositions that elicit the anti-viral immune response can be combined in a polyvalent immunogenic composition administered to a subject in a single immunization step, or they can be administered separately (in monovalent immunogenic compositions) in a coordinate (or prime-boost) immunization protocol.

There can be several boosts, and each boost can be a different disclosed immunogen. In some examples that the boost may be the same immunogen as another boost, or the prime. The prime and boost can be administered as a single dose or multiple doses, for example two doses, three doses, four doses, five doses, six doses or more can be administered to a subject over days, weeks or months. Multiple boosts can also be given, such one to five (e.g., 1, 2, 3, 4 or 5 boosts), or more. Different dosages can be used in a series of sequential immunizations. For example a relatively large dose in a primary immunization and then a boost with relatively smaller doses.

In some embodiments, the boost can be administered about two, about three to eight, or about four, weeks following the prime, or about several months after the prime. In some embodiments, the boost can be administered about 5, about 6, about 7, about 8, about 10, about 12, about 18, about 24, months after the prime, or more or less time after the prime. Periodic additional boosts can also be used at appropriate time points to enhance the subject's “immune memory.” The adequacy of the vaccination parameters chosen, e.g., formulation, dose, regimen and the like, can be determined by taking aliquots of scrum from the subject and assaying antibody titers during the course of the immunization program. In addition, the clinical condition of the subject can be monitored for the desired effect, e.g., inhibition of NiV infection or improvement in disease state (e.g., reduction in viral load). If such monitoring indicates that vaccination is sub-optimal, the subject can be boosted with an additional dose of immunogenic composition, and the vaccination parameters can be modified in a fashion expected to potentiate the immune response.

In some embodiments, the prime-boost method can include DNA-primer and protein-boost vaccination protocol to a subject. The method can include two or more administrations of the nucleic acid molecule or the protein.

For protein therapeutics, typically, each human dose will comprise 1-1000 μg of protein, such as from about 1 μg to about 100 μg, for example, from about 1 μg to about 50 μg, such as about 1 μg, about 2 μg, about 5 μg, about 10 μg, about 15 μg, about 20 μg, about 25 μg, about 30 μg, about 40 μg, or about 50 μg. The amount utilized in an immunogenic composition is selected based on the subject population (e.g., infant or elderly). An optimal amount for a particular composition can be ascertained by standard studies involving observation of antibody titers and other responses in subjects. It is understood that a therapeutically effective amount of a disclosed immunogen, such as a recombinant NiV F ectodomain or immunogenic fragment thereof, viral vector, or nucleic acid molecule in a immunogenic composition, can include an amount that is ineffective at eliciting an immune response by administration of a single dose, but that is effective upon administration of multiple dosages, for example in a prime-boost administration protocol.

Upon administration of a disclosed immunogen the immune system of the subject typically responds to the immunogenic composition by producing antibodies specific for viral protein. Such a response signifies that an immunologically effective dose was delivered to the subject.

For each particular subject, specific dosage regimens can be evaluated and adjusted over time according to the individual need and professional judgment of the person administering or supervising the administration of the immunogenic composition. The dosage and number of doses will depend on the setting, for example, in an adult or anyone primed by prior NiV infection or immunization, a single dose may be a sufficient booster. In naïve subjects, in some examples, at least two doses would be given, for example, at least three doses. In some embodiments, an annual boost is given, for example, along with an annual influenza vaccination.

In some embodiments, the antibody response of a subject will be determined in the context of evaluating effective dosages/immunization protocols. In most instances it will be sufficient to assess the antibody titer in serum or plasma obtained from the subject. Decisions as to whether to administer booster inoculations and/or to change the amount of the therapeutic agent administered to the individual can be at least partially based on the antibody titer level. The antibody titer level can be based on, for example, an immunobinding assay which measures the concentration of antibodies in the serum which bind to an antigen including, for example, an NiV F protein.

Determination of effective dosages is typically based on animal model studies followed up by human clinical trials and is guided by administration protocols that significantly reduce the occurrence or severity of targeted disease symptoms or conditions in the subject, or that induce a desired response in the subject (such as a neutralizing immune response). Suitable models in this regard include, for example, murine, rat, porcine, feline, ferret, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer a therapeutically effective amount of the composition (for example, amounts that are effective to elicit a desired immune response or alleviate one or more symptoms of a targeted disease). In alternative embodiments, an effective amount or effective dose of the composition may simply inhibit or enhance one or more selected biological activities correlated with a disease or condition, as set forth herein, for either therapeutic or diagnostic purposes.

Administration of an immunogenic composition that elicits an immune response to reduce or prevent an infection, can, but does not necessarily completely, eliminate such an infection, so long as the infection is measurably diminished. For example, administration of an effective amount of the agent can decrease the NiV infection (for example, as measured by infection of cells, or by number or percentage of subjects infected by NiV by a desired amount, for example by at least 10%, at least 20%, at least 50%, at least 60%, at least 70%, at least 80%, at least 90%, at least 95%, at least 98%, or even at least 100% (elimination or prevention of detectable NiV infection, as compared to a suitable control.

In some embodiments, administration of a therapeutically effective amount of one or more of the disclosed immunogens to a subject induces a neutralizing immune response in the subject. To assess neutralization activity, following immunization of a subject, serum can be collected from the subject at appropriate time points, frozen, and stored for neutralization testing. Methods to assay for neutralization activity include, but are not limited to, plaque reduction neutralization (PRNT) assays, microneutralization assays, flow cytometry based assays, single-cycle infection assays. In some embodiments, the serum neutralization activity can be assayed using a panel of NiV pseudoviruses.

One approach to administration of nucleic acids is direct immunization with plasmid DNA, such as with a mammalian expression plasmid. Immunization by nucleic acid constructs is well known in the art and taught, for example, in U.S. Pat. No. 5,643,578 (which describes methods of immunizing vertebrates by introducing DNA encoding a desired antigen to elicit a cell-mediated or a humoral response), and U.S. Pat. Nos. 5,593,972 and 5,817,637 (which describe operably linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids (or of the synthetic peptides themselves), and immune-stimulating constructs, or ISCOMS™, negatively charged cage-like structures of 30-40 nm in size formed spontaneously on mixing cholesterol and Quil A™ (saponin). Protective immunity has been generated in a variety of experimental models of infection, including toxoplasmosis and Epstein-Barr virus-induced tumors, using ISCOMS™ as the delivery vehicle for antigens (Mowat and Donachie, Immunol. Today 12:383, 1991). Doses of antigen as low as 1 μg encapsulated in ISCOMS™ have been found to produce Class I mediated CTL responses (Takahashi et al., Nature 344:873, 1990).

In some embodiments, a plasmid DNA vaccine is used to express a disclosed immunogen in a subject. For example, a nucleic acid molecule encoding a disclosed immunogen can be administered to a subject to elicit an immune response to the F protein of NiV. In some embodiments, the nucleic acid molecule can be included on a plasmid vector for DNA immunization, such as the pVRC8400 vector (described in Barouch et al., J. Virol, 79, 8828-8834, 2005, which is incorporated by reference herein).

In another approach to using nucleic acids for immunization, a disclosed immunogen can be expressed by attenuated viral hosts (such as an attenuated NiV vector) or vectors or bacterial vectors. Recombinant vaccinia virus, adeno-associated virus (AAV), herpes virus, retrovirus, cytogmeglovirus or other viral vectors can be used to express the peptide or protein, thereby eliciting a CTL response. For example, vaccinia vectors and methods useful in immunization protocols are described in U.S. Pat. No. 4,722,848. BCG (Bacillus Calmette Guerin) provides another vector for expression of the peptides (see Stover, Nature 351:456-460, 1991).

In another example, a disclosed immunogen can be administered to a subject using RNA immunization, such as with a lipid-encapsulated mRNA immunization platform (see, e.g., Roth et al., “A Modified mRNA Vaccine Targeting Immunodominant NS Epitopes Protects Against Dengue Virus Infection in HLA Class I Transgenic Mice,” Frot Immunol., Jun. 21, 2019, Vol. 10, Article 1424; Jagger et al., J Infect Dis, “Protective Efficacy of Nucleic Acid Vaccines Against Transmission of Zika Virus During Pregnancy in Mice,” jiz338, Jul. 1, 2019; Feldman et al., “mRNA vaccines against H10N8 and H7N9 influenza viruses of pandemic potential are immunogenic and well tolerated in healthy adults in phase 1 randomized clinical trials,” Vaccine, 37(25), 3326-3334, 2019; and Hasset et al., “Optimization of Lipid Nanoparticles for Intramuscular Administration of mRNA Vaccines,” Mol Ther Nucleic Acids, 15: 1-11, 2019.

In one embodiment, a nucleic acid encoding a protomer of a disclosed NiV F ectodomain trimer is introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

### EXAMPLES

The following examples are provided to illustrate particular features of certain embodiments, but the scope of the claims should not be limited to those features exemplified.

### Example 1

### NiV F Proteins Stabilized in a Prefusion Conformation

The example illustrates embodiments of a NiV F ectodomain trimer stabilized in a prefusion conformation by one or more amino acid substitutions. The prefusion-stabilized NiV F ectodomain trimers are useful, for example, for inducing a neutralizing immune response to NiV in a subject.

When initially produced in cells, the NiV F ectodomain linked to a C-terminal GCN4 trimerization domain forms trimers that are mostly in the prefusion conformation. However, when stored at 4° C., the metastable trimers undergo a progressive structural transformation to the NiV F postfusion conformation.

Accordingly, structure-based vaccine design was used to identify mutations for the stabilization of the NiV F ectodomain in a prefusion conformation (based on prefusion NiV F structure PDB ID 2B9B), and also to eliminate the F₁/F₂ cleavage site to produce a “single chain NiV F protein with increased expression. Several different stabilization strategies were employed to “lock” the NiV F ectodomain in the prefusion conformation, including introduction of disulfide bonds, cavity-filling amino acid substitutions, and proline substitutions. In total, approximately 150 different mutants were designed, expressed, purified, and assessed for expression level and binding to antibody 5B3, which is specific for the prefusion conformation of NiV F. Of over 150 constructs tested, the following showed the best combination of prefusion stabilization and protein expression: NiV05, NiV07, NiV08, NiV09, NiV11, NiV12, NiV13, NiV14, NiV15, and NiV16.

The mutations were introduced into a NiV F ectodomain linked to a C-terminal GCN4 trimerization domain, and the resulting mutants were screened in a 96-well microculture high-throughput mini-expression and ELISA assay using prefusion NiV F antibody 5B3. Approximately 150 constructs were produced and expressed, including:

### Wildtype Ectodomain Linked to GCN4 Trimerization Domain

NiV01: NIV F (22-497) GCN4
   Single Chain Ectodomain with Fusion of F₂/F₁ and Linkage to GCN4 Trimerization Domain
NiV02: NiV F (22-497) GCN4, 4101-116, residues N100-G117 linked by a serine
NiV03: NiV F (22-497) GCN4, A100-115, residues N99-A116 linked by a GSG linker
NiV04: NiV F (22-497) GCN4, 4102-113, residues T101-I114 linked by a GSG linker
NiV06: NiV F (22-497) GCN4, 4100-116, residues N99-G117 linked by a GGS linker
NiV10: NiV F (22-497) GCN4, A100-116, residues N99-G117 directly linked

### Prefusion Stabilized Ectodomain Linked to GCN4 Trimerization Domain

NiV05: NiV F (22-497) GCN4, intraprotomer L104C-I114C disulfide
NiV07: NiV F (22-497) GCN4, intraprotomer I114C-I426C disulfide
NiV08: NiV F (22-497) GCN4, L172F cavity filling substitution
NiV09: NiV F (22-497) GCN4, S191P
NiV11: NiV F (22-497) GCN4, Y178W cavity filling substitution
NiV12: NiV F (22-497) GCN4, intraprotomer A130C-V222C disulfide
NiV13: NiV F (22-497) GCN4, Q70G
NiV14: NiV F (22-497) GCN4, D188G, S191G
NiV15: NiV F (22-497) GCN4, intraprotomer Q162C-T168C disulfide
NiV16: NiV F (22-497) GCN4, I228F

Expression and purification of the single chain and prefusion-stabilized NiV F proteins showed a substantial increase in expression level compared to corresponding the unmodified NiV F (NiV01):

| Construct | mg/L |
|---|---|
| NiV01 | 0.1 |
| NiV02 | 11.6 |
| NiV03 | 5.7 |
| NiV04 | 9.9 |
| NiV05 | 5.6 |
| NiV06 | 11.5 |
| NiV07 | 5.8 |
| NiV08 | 0.8 |
| NiV09 | 1.1 |
| NiV10 | 4.9 |
| NiV11 | 1.2 |
| NiV12 | 0.4 |
| NiV13 | 0.6 |
| NiV14 | 1.0 |
| NiV15 | 1.0 |
| NiV16 | 1.1 |

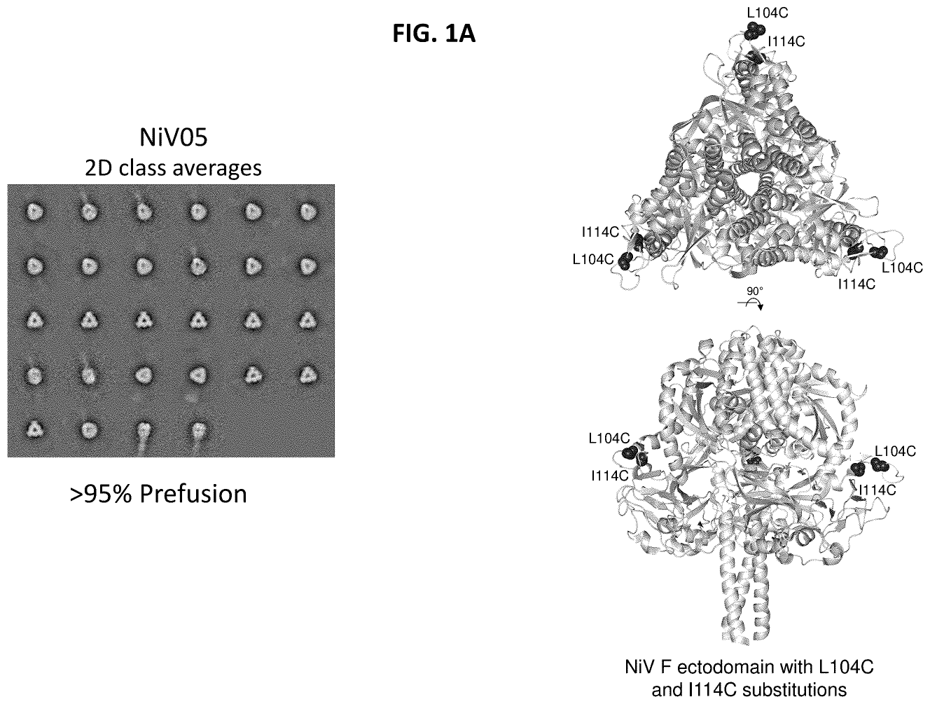

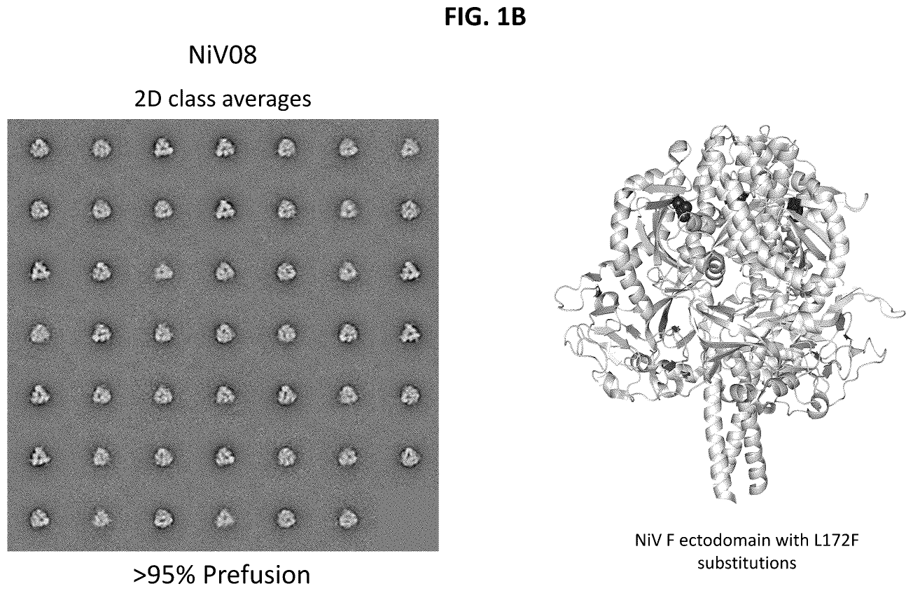

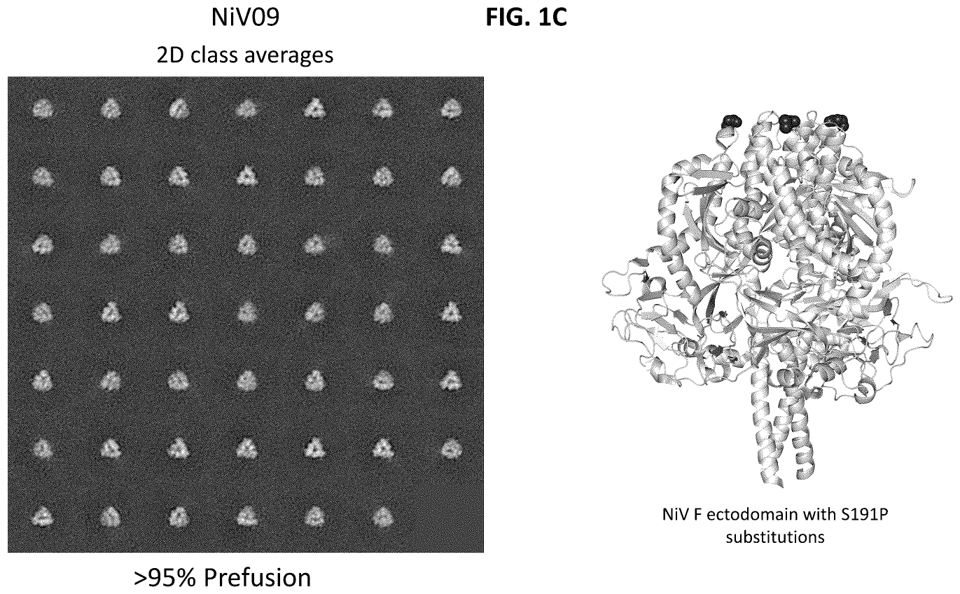

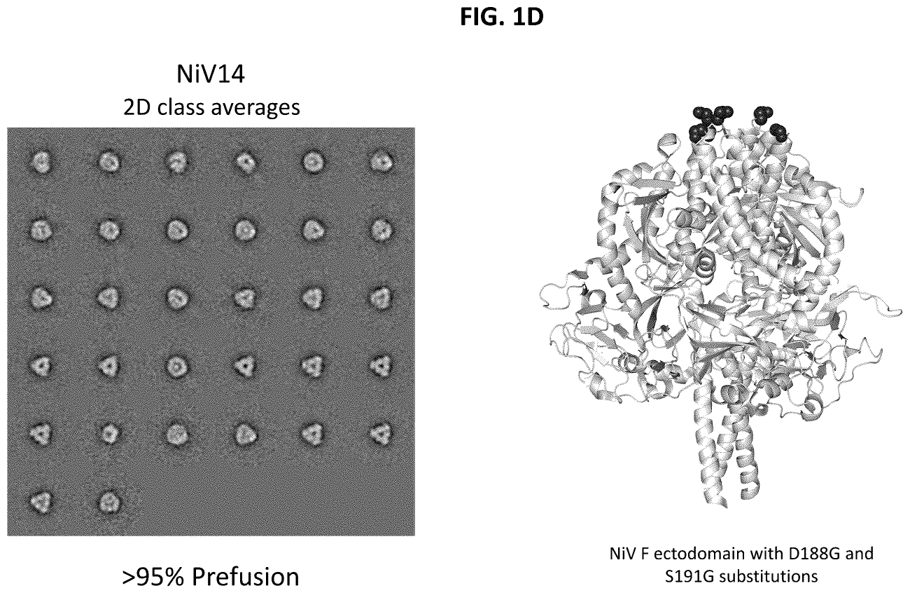

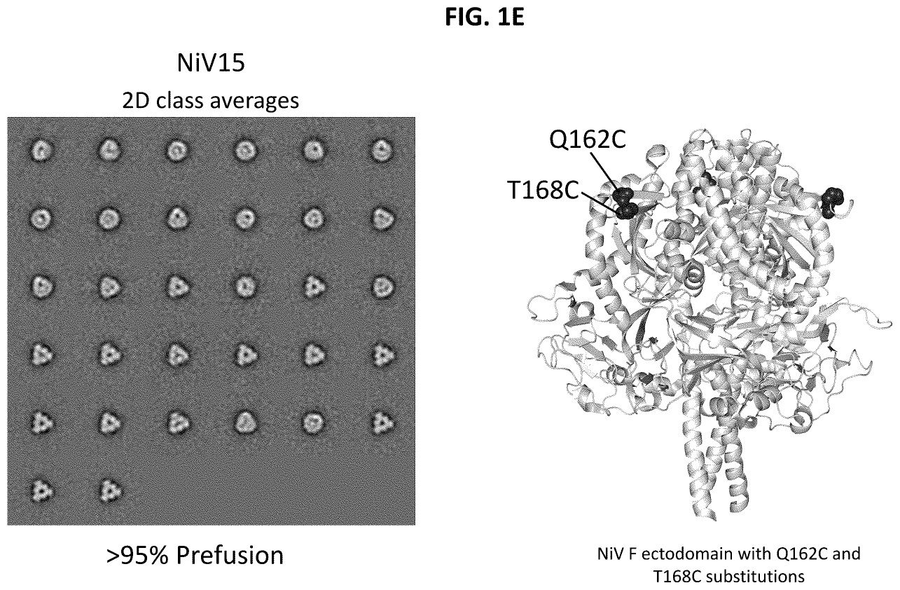

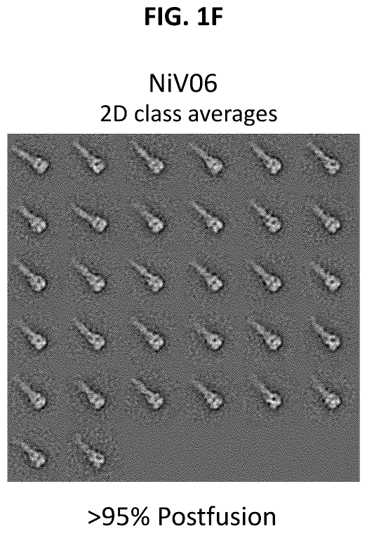

Negative stain EM was used to confirm the conformation of the NiV05, NiV07, NiV08, NiV09, NiV11, NiV12, NiV13, NiV14, NiV15, and NiV16 F variants. Exemplary images are provided in FIGS. 1A-IF. FIG. 1F shows that the NiV06 construct, which has a single-chain mutation, but no prefusion stabilization mutations is in the postfusion conformation.

Next, combinations of mutations for prefusion stabilization and protein production were also assessed. Of the many combinations tested, the following showed the best combination of prefusion stabilization and protein expression:
NiVop01: NiV F (22-497) GCN4, I114C-I426C, L172F
NiVop02: NiV F (22-497) GCN4, L104C-I114C, L172F
NiVop03: NiV F (22-497) GCN4, 4102-113, T101-I114 linked by GSG, L172F
NiVop04: NiV F (22-497) GCN4, I114C-I426C, S191P
NiVop05: NiV F (22-497) GCN4, L104C-I114C, S191P
NiVop06: NiV F (22-497) GCN4, 4102-113, T101-I114 linked by GSG, S191P
NiVop07: NiV F (22-497) GCN4, I114C-I426C, L172F, S191P
NiVop08: NiV F (22-497) GCN4, L104C-I114C, L172F, S191P
NiVop09: NiV F (22-497) GCN4, 4102-113, T101-I114 linked by GSG, L172P, S191P
NiVop12: NiV F (22-497) GCN4, L172P, S191P
NiVop13: NiV F (22-497) GCN4, L172P, S191P, Q70G
NiVop14: NiV F (22-497) GCN4, L104C-I114C, L172F, S191P, Q70G
NiVop15: NiV F (22-497) GCN4, L104C-I114C, L172F, Q70G
NiVop16: NiV F (22-497) GCN4, L104C-I114C, Q70G
NiVop17: NiV F (22-497) GCN4, L104C-I114C, Q162C-T168C, L172F, S191P
NiVop18: NiV F (22-497) GCN4, L104C-I114C, A130C-V222C, L172F, S191P

These constructs were expressed in cells, purified, and assessed for 5B3 binding. All of the purified proteins bound to 5B3, indicated that they were in the prefusion conformation. Further, all of these constructs showed a substantial increase in expression level compared to corresponding unmodified NiV F:

| Construct | mg/L |
|---|---|
| NiV01 | 0.1 |
| NiVop01 | 3.9 |
| NiVop02 | 4.0 |
| NiVop03 | 5.8 |
| NiVop04 | 2.8 |
| NiVop05 | 2.2 |
| NiVop06 | 5.6 |
| NiVop07 | 2.8 |
| NiVop08 | 5.6 |
| NiVop09 | 4.9 |
| NiVop12 | 0.6 |
| NiVop13 | 0.6 |
| NiVop14 | 1.6 |
| NiVop15 | 2.9 |
| NiVop16 | 1.3 |

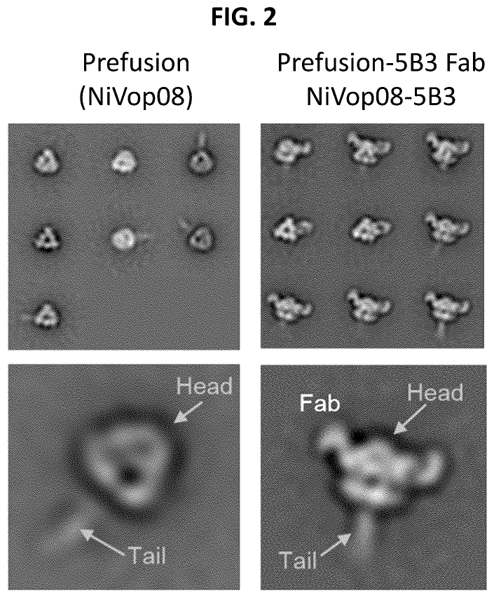

Negative stain EM showed that all of these constructs were in a prefusion-specific conformation. Exemplary negative stain EM images for NiVop08 alone and bound by 5B3 Fab are shown in FIG. 2.

As illustrated in FIGS. 1-2, negative EM can be used to distinguish NiV F ectodomain trimers that are in the prefusion conformation from those that are in the postfusion conformation.

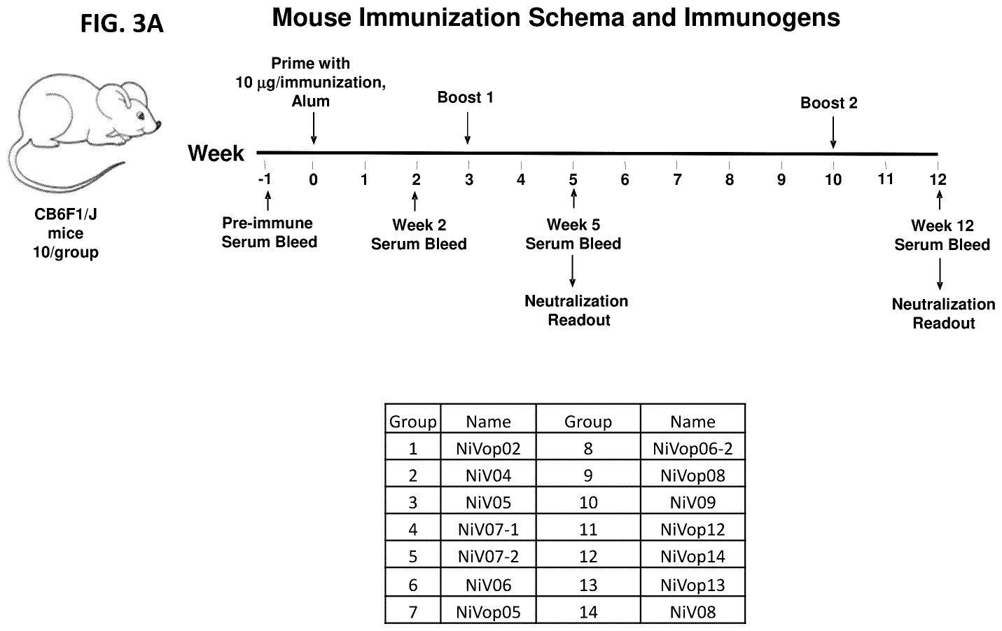

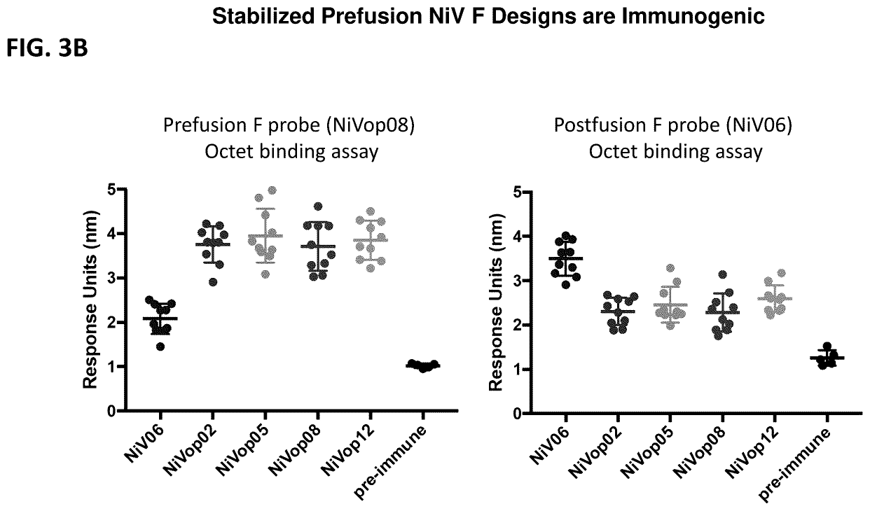

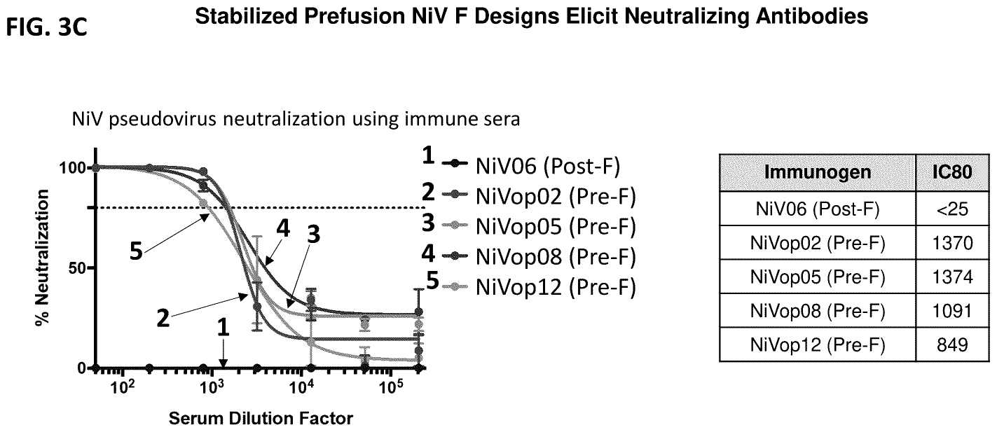

Immunization assays were conducted with several of the modified NiV F ectodomain trimers to determine if these trimers could produce a neutralizing immune response in an animal model. CB6F1/J mice were immunized with 10 μg of NiV F in Alum at weeks 0, 3, and 10, and the neutralization titer of sera from the immunized mice was assayed as weeks 5 and 12 (see FIG. 3A). Sera from immunized mice was tested for binding to prefusion-stabilized NiV F ectodomain trimer (NiVop08) and postfusion NiV F ectodomain trimer (NiV06) using an Octet binding assay (FIG. 3B). The NiVop08 or NiV06 trimer was linked to the sensor and sera from the indicated immunizations assayed for binding. Sera from NiVop02, NiVop05, NiVop08, and NiVop12 immunized animals bound preferentially to prefusion F (NiVop08) relative to postfusion F (NiV06). In contrast, sera from NiV06 immunized animals bound preferentially to postfusion F (NiV06) relative to prefusion F (NiVop08).

The immune sera was assessed in a NiV neutralization assay (FIG. 3C), which showed that immune sera from animals treated with the prefusion NiV F trimer neutralized NiV. Thus, immunization with soluble prefusion-stabilized NiV F ectodomain trimer elicited a neutralizing immune response in an animal model.

### Summary of Methods

Protein expression and purification. NiV F mutations were made by site-directed mutagenesis using the Stratagene Quik-change procedure. NiV F variants were expressed by transient transfection of Expi293F cells with plasmid DNA encoding a precursor of the protomer of the variant NiV F trimer. Cell culture supernatants were harvested 5 days post transfection and centrifuged at 10,000 g to remove cell debris. The supernatants were sterile-filtered, and NiV F proteins were purified by nickel and streptactin-affinity chromatography followed by size-exclusion chromatography. The nickel and streptactin purification tags were removed for animal immunization.

Screening of prefusion-stabilized NiV F constructs. Prefusion NiV F variants were derived from the native NiV F sequences. A 96-well microplate-formatted transient gene expression approach was used to achieve high-throughput expression of various NiV F proteins using a previously described high-throughput assay developed for HIV (Pancera et al., PloS one, 8, c55701, 2013). Briefly, 24 h prior to transfection HEK 293T cells were seeded in each well of a 96-well microplate at a density of 2.5×10⁵ cells/ml in expression medium (high glucose DMEM supplemented with 10% ultra-low IgG fetal bovine serum and 1×-non-essential amino acids), and incubated at 37° ° C., 5% CO2 for 20 h. Plasmid DNA encoding a precursor of the protomer of the variant NiV F trimer and TrucFect-Max (United BioSystems, MD) were mixed and added to the growing cells, and the 96-well plate incubated at 37° C., 5% CO2. One day post transfection, enriched medium (high glucose DMEM plus 25% ultra-low IgG fetal bovine serum, 2× nonessential amino acids, 1× glutamine) was added to each well, and the 96-well plate was returned to the incubator for continuous culture. On day five, post transfection, supernatants with the expressed NiV F variants were harvested and tested by ELISA for binding to prefusion specific antibody 53B using Ni2+-NTA microplates.

Negative stain electron microscopy. Samples were adsorbed to freshly glow-discharged carbon-film grids, rinsed twice with buffer and stained with freshly made 0.75% uranyl formate. Images were recorded on an FEI T20 microscope with a 2k×2k Eagle CCD camera at a pixel size of 1.5 Å. Image analysis and 2D averaging was performed with Bsoft (Heymann and Belnap, J. Struct Biol., 157, 3, 2007) and EMAN (Ludtke, Baldwin, and Chiu, J. Struct. Biol., 128, 82, 1999).

Mouse immunizations. All animal experiments were reviewed and approved by the Animal Care and Use Committee of the Vaccine Research Center, NIAID, NIH, and all animals were housed and cared for in accordance with local, state, federal, and institute policies in an American Association for Accreditation of Laboratory Animal Care (AAALAC)-accredited facility at the NIH. Hybrid mice that were the first filial offspring of a cross between BALB/c females (C) and C57BL/6J males (B6) (The Jackson Laboratory) known as CB6F1/J at ages 6 weeks to 12 weeks were intramuscularly injected with NiV F ectodomain trimer immunogens at week 0, week 3, and week 10. The frozen NiV F ectodomain trimer variant immunogen proteins were thawed on ice and mixed with Alum adjuvant at 10 μg NiV F per animal per immunization, with injections taking place within 1 h of immunogen:adjuvant preparation. No adverse effect from immunization was observed. Blood was collected at least three days before immunization, and at week two, week five and week 12 post initial immunization.

Generation of NiV Pseudotypes. To obtain VSV□G-luciferase pseudotyped with NiVF and G proteins, BHK21 cells were first cotransfected with VRC8400 NiVF_{WT} and VRC8400 NiVG. Transfected cells showing extensive cell-to-cell fusion were infected with VSVG complemented VSV□G-luciferase. At 1 hour post-infection, the input virus was removed and DMEM containing 10% FBS was added to the cells. Medium containing VSV□G-luciferase pseudotyped with NiVF_{WT} and G was collected between 24-36 hours and titered on Vero76 cells with anti-VSVG antibody measuring luciferase activity.

NiV neutralization assays. To measure NiV neutralizing antibodies in serum, VSVΔG-luciferase/NiVF-G pseudotypes were first incubated with anti-VSV G 8G5 antibody at 5 μg/mL for 30 minutes at room temperature to neutralize any trace infection due to residual VSV G that may have incorporated into the particles pseudotyped with NiVF_{WT} and NiV G proteins. Pooled serum samples from each immunization group were serially diluted (1:100-1:12800) in DMEM/10% FBS and mixed 1:1 with appropriate amount of pseudotype particles. The mixture was incubated at room temperature for 30 minutes and 50 μL of each dilution was transferred to a monolayer of Vero 76 cells grown in a 96-well plate (in triplicate). Cells were incubated for 20-24 hours at 37° C. Cells were lysed in 20 μL of cell culture lysis buffer. Luciferase assay reagent was added to the cell lysate prior to measuring luciferase activity. The IC₅₀ for each sample was calculated by curve fitting and non-linear regression using GraphPad Prism (GraphPad Software Inc., CA)

Sera antigenic analysis. Mouse sera from the immunization groups were assessed for binding to pre- and post-fusion NiV F ectodomain trimers using a ForteBio Octet HTX instrument. Week 5 sera were diluted 1:400 in 1% BSA/PBS. Anti penta His, (HIS1K) sensor tips obtained from FortéBio were equilibrated in PBS prior to running an assay. NiV F trimeric protein at 20 μg/ml in 1% BSA/PBS was loaded onto HIS1K biosensors using the C-terminal His tag for 300s. HIS1K tips loaded with pre or postfusion NiV F trimers were equilibrated for 60s in 1% BSA/PBS followed by a serum association step for 300s and a subsequent dissociation step for an additional 300s. Data analysis was performed using Octet and GraphPad Prism 6 software.

### Sequences:

NiV01
(SEQ ID NO: 1)
mysmqlascvtltlvllvnsQGILHYEKLSKIGLVKGVTRKYKIKSNPLT
KDIVIKMIPNVSDMSQCTGSVMENYKTRLNGILTPIKGALEIYKNNTHDL
VGDVRLAGVIMAGVAIGIATAAQITAGVALYEAMKNADNINKLKSSIEST
NEAVVKLQETAEKTVYVLTALQDYINTNLVPTIDKISCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
DSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVIISLGKYLGSVNYNSEGIAIGPPVFTDKV
DISSQISSMNQSLQQSKDYIKEAQRLLDTVNPSLklmkqiedkieeilsk
iyhieneiarikkligeapgglvprgshhhhhhsawshpqfek

NiV02
(SEQ ID NO: 2)
mysmqlascvtltlvllvnsQGILHYEKLSKIGLVKGVTRKYKIKSNPLT
KDIVIKMIPNVSNMSQCTGSVMENYKTRLNGILTPIKGALEIYKNNSGVA
IGIATAAQITAGVALYEAMKNADNINKLKSSIESTNEAVVKLQETAEKTV
YVLTALQDYINTNLVPTIDKISCKQTELSLDLALSKYLSDLLFVFGPNLQ
DPVSNSMTIQAISQAFGGNYETLLRTLGYATEDFDDLLESDSITGQIIYV
DLSSYYIIVRVYFPILTEIQQAYIQELLPVSFNNDNSEWISIVPNFILVR
NTLISNIEIGFCLITKRSVICNQDYATPMTNNMRECLTGSTEKCPRELVV
SSHVPRFALSNGVLFANCISVTCQCQTTGRAISQSGEQTLLMIDNTTCPT
AVLGNVIISLGKYLGSVNYNSEGIAIGPPVFTDKVDISSQISSMNQSLQQ
SKDYIKEAQRLLDTVNPSLklmkqiedkieeilskiyhieneiarikkli
geapgglvprgshhhhhhsawshpqfek

NiV03
(SEQ ID NO: 3)
mysmqlascvtltlvllvnsQGILHYEKLSKIGLVKGVTRKYKIKSNPLT
KDIVIKMIPNVSNMSQCTGSVMENYKTRLNGILTPIKGALEIYKNGSGAG
VAIGIATAAQITAGVALYEAMKNADNINKLKSSIESTNEAVVKLQETAEK
TVYVLTALQDYINTNLVPTIDKISCKQTELSLDLALSKYLSDLLFVFGPN
LQDPVSNSMTIQAISQAFGGNYETLLRTLGYATEDFDDLLESDSITGQII
YVDLSSYYIIVRVYFPILTEIQQAYIQELLPVSFNNDNSEWISIVPNFIL
VRNTLISNIEIGFCLITKRSVICNQDYATPMTNNMRECLTGSTEKCPREL
VVSSHVPRFALSNGVLFANCISVTCQCQTTGRAISQSGEQTLLMIDNTTC
PTAVLGNVIISLGKYLGSVNYNSEGIAIGPPVFTDKVDISSQISSMNQSL
QQSKDYIKEAQRLLDTVNPSLklmkqiedkieeilskiyhieneiarikk
ligeapgglvprgshhhhhhsawshpqfek

NiV04
(SEQ ID NO: 4)
mysmqlascvtltlvllvnsQGILHYEKLSKIGLVKGVTRKYKIKSNPLT
KDIVIKMIPNVSNMSQCTGSVMENYKTRLNGILTPIKGALEIYKNNTGSG
IMAGVAIGIATAAQITAGVALYEAMKNADNINKLKSSIESTNEAVVKLQE
TAEKTVYVLTALQDYINTNLVPTIDKISCKQTELSLDLALSKYLSDLLFV
FGPNLQDPVSNSMTIQAISQAFGGNYETLLRTLGYATEDFDDLLESDSIT
GQIIYVDLSSYYIIVRVYFPILTEIQQAYIQELLPVSFNNDNSEWISIVP
NFILVRNTLISNIEIGFCLITKRSVICNQDYATPMTNNMRECLTGSTEKC
PRELVVSSHVPRFALSNGVLFANCISVTCQCQTTGRAISQSGEQTLLMID
NTTCPTAVLGNVIISLGKYLGSVNYNSEGIAIGPPVFTDKVDISSQISSM
NQSLQQSKDYIKEAQRLLDTVNPSLklmkqiedkieeilskiyhieneia
rikkligeapgglvprgshhhhhhsawshpqfek

NiV05
(SEQ ID NO: 5)
mysmqlascvtltlvllvnsQGILHYEKLSKIGLVKGVTRKYKIKSNPLT
KDIVIKMIPNVSNMSQCTGSVMENYKTRLNGILTPIKGALEIYKNNTHDC
VGDVRLAGVCMAGVAIGIATAAQITAGVALYEAMKNADNINKLKSSIEST
NEAVVKLQETAEKTVYVLTALQDYINTNLVPTIDKISCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVIISLGKYLGSVNYNSEGIAIGPPVFTDKV
DISSQISSMNQSLQQSKDYIKEAQRLLDTVNPSLklmkqiedkieeilsk
iyhieneiarikkligeapgglvprgshhhhhhsawshpqfek

NiV06
(SEQ ID NO: 6)
mysmqlascvtltlvllvnsQGILHYEKLSKIGLVKGVTRKYKIKSNPLT
KDIVIKMIPNVSNMSQCTGSVMENYKTRLNGILTPIKGALEIYKNGGSGV
AIGIATAAQITAGVALYEAMKNADNINKLKSSIESTNEAVVKLQETAEKT
VYVLTALQDYINTNLVPTIDKISCKQTELSLDLALSKYLSDLLFVFGPNL
QDPVSNSMTIQAISQAFGGNYETLLRTLGYATEDFDDLLESDSITGQIIY
VDLSSYYIIVRVYFPILTEIQQAYIQELLPVSFNNDNSEWISIVPNFILV
RNTLISNIEIGFCLITKRSVICNQDYATPMTNNMRECLTGSTEKCPRELV
VSSHVPRFALSNGVLFANCISVTCQCQTTGRAISQSGEQTLLMIDNTTCP
TAVLGNVIISLGKYLGSVNYNSEGIAIGPPVFTDKVDISSQISSMNQSLQ
QSKDYIKEAQRLLDTVNPSLklmkqiedkieeilskiyhieneiarikkl
igeapgglvprgshhhhhhsawshpqfek

NiV07
(SEQ ID NO: 7)
mysmqlascvtltlvllvnsQGILHYEKLSKIGLVKGVTRKYKIKSNPLT
KDIVIKMIPNVSNMSQCTGSVMENYKTRLNGILTPIKGALEIYKNNTHDL
VGDVRLAGVCMAGVAIGIATAAQITAGVALYEAMKNADNINKLKSSIEST
NEAVVKLQETAEKTVYVLTALQDYINTNLVPTIDKISCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVCISLGKYLGSVNYNSEGIAIGPPVFTDKV
DISSQISSMNQSLQQSKDYIKEAQRLLDTVNPSLklmkqiedkieeilsk
iyhieneiarikkligeapgglvprgshhhhhhsawshpqfek

NiV08
(SEQ ID NO: 8)
mysmqlascvtltlvllvnsQGILHYEKLSKIGLVKGVTRKYKIKSNPLT
KDIVIKMIPNVSDMSQCTGSVMENYKTRLNGILTPIKGALEIYKNNTHDL
VGDVRLAGVIMAGVAIGIATAAQITAGVALYEAMKNADNINKLKSSIEST
NEAVVKLQETAEKTVYVFTALQDYINTNLVPTIDKISCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
DSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVIISLGKYLGSVNYNSEGIAIGPPVFTDKV
DISSQISSMNQSLQQSKDYIKEAQRLLDTVNPSLklmkqiedkieeilsk
iyhieneiarikkligeapgglvprgshhhhhhsawshpqfek

NiV09
(SEQ ID NO: 9)
mysmqlascvtltlvllvnsQGILHYEKLSKIGLVKGVTRKYKIKSNPLT
KDIVIKMIPNVSNMSQCTGSVMENYKTRLNGILTPIKGALEIYKNNTHDL
VGDVRLAGVIMAGVAIGIATAAQITAGVALYEAMKNADNINKLKSSIEST
NEAVVKLQETAEKTVYVLTALQDYINTNLVPTIDKIPCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVIISLGKYLGSVNYNSEGIAIGPPVFTDKV
DISSQISSMNQSLQQSKDYIKEAQRLLDTVNPSLklmkqiedkieeilsk
iyhieneiarikkligeapgglvprgshhhhhhsawshpqfek

NiV10
(SEQ ID NO: 10)
mysmqlascvtltlvllvnsQGILHYEKLSKIGLVKGVTRKYKIKSNPLT
KDIVIKMIPNVSNMSQCTGSVMENYKTRLNGILTPIKGALEIYKNGVAIG
IATAAQITAGVALYEAMKNADNINKLKSSIESTNEAVVKLQETAEKTVYV
LTALQDYINTNLVPTIDKISCKQTELSLDLALSKYLSDLLFVFGPNLQDP
VSNSMTIQAISQAFGGNYETLLRTLGYATEDFDDLLESDSITGQIIYVDL
SSYYIIVRVYFPILTEIQQAYIQELLPVSFNNDNSEWISIVPNFILVRNT
LISNIEIGFCLITKRSVICNQDYATPMTNNMRECLTGSTEKCPRELVVSS
HVPRFALSNGVLFANCISVTCQCQTTGRAISQSGEQTLLMIDNTTCPTAV
LGNVIISLGKYLGSVNYNSEGIAIGPPVFTDKVDISSQISSMNQSLQQSK
DYIKEAQRLLDTVNPSLklmkqiedkieeilskiyhieneiarikklige
apgglvprgshhhhhhsawshpqfek

NiV11
(SEQ ID NO: 11)
mysmqlascvtltlvllvnsQGILHYEKLSKIGLVKGVTRKYKIKSNPLT
KDIVIKMIPNVSNMSQCTGSVMENYKTRLNGILTPIKGALEIYKNNTHDL
VGDVRLAGVIMAGVAIGIATAAQITAGVALYEAMKNADNINKLKSSIEST
NEAVVKLQETAEKTVYVLTALQDWINTNLVPTIDKISCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVIISLGKYLGSVNYNSEGIAIGPPVFTDKV
DISSQISSMNQSLQQSKDYIKEAQRLLDTVNPSLklmkqiedkieeilsk
iyhieneiarikkligeapgglvprgshhhhhhsawshpqfek

NiV12
(SEQ ID NO: 12)
mysmqlascvtltlvllvnsQGILHYEKLSKIGLVKGVTRKYKIKSNPLT
KDIVIKMIPNVSNMSQCTGSVMENYKTRLNGILTPIKGALEIYKNNTHDL
VGDVRLAGVIMAGVAIGIATAAQITCGVALYEAMKNADNINKLKSSIEST
NEAVVKLQETAEKTVYVLTALQDYINTNLVPTIDKISCKQTELSLDLALS
KYLSDLLFVFGPNLQDPCSNSMTIQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVIISLGKYLGSVNYNSEGIAIGPPVFTDKV
DISSQISSMNQSLQQSKDYIKEAQRLLDTVNPSLklmkqiedkieeilsk
iyhieneiarikkligeapgglvprgshhhhhhsawshpqfek

NiV13
(SEQ ID NO: 13)
mysmqlascvtltlvllvnsQGILHYEKLSKIGLVKGVTRKYKIKSNPLT
KDIVIKMIPNVSNMSGCTGSVMENYKTRLNGILTPIKGALEIYKNNTHDL
VGDVRLAGVIMAGVAIGIATAAQITAGVALYEAMKNADNINKLKSSIEST
NEAVVKLQETAEKTVYVLTALQDYINTNLVPTIDKISCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVIISLGKYLGSVNYNSEGIAIGPPVFTDKV
DISSQISSMNQSLQQSKDYIKEAQRLLDTVNPSLklmkqiedkieeilsk
iyhieneiarikkligeapgglvprgshhhhhhsawshpqfek

NiV14
(SEQ ID NO: 14)
mysmqlascvtltlvllvnsQGILHYEKLSKIGLVKGVTRKYKIKSNPLT
KDIVIKMIPNVSNMSQCTGSVMENYKTRLNGILTPIKGALEIYKNNTHDL
VGDVRLAGVIMAGVAIGIATAAQITAGVALYEAMKNADNINKLKSSIEST
NEAVVKLQETAEKTVYVLTALQDYINTNLVPTIGKIGCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVIISLGKYLGSVNYNSEGIAIGPPVFTDKV
DISSQISSMNQSLQQSKDYIKEAQRLLDTVNPSLklmkqiedkieeilsk
iyhieneiarikkligeapgglvprgshhhhhhsawshpqfek

NiV15
(SEQ ID NO: 15)
mysmqlascvtltlvllvnsQGILHYEKLSKIGLVKGVTRKYKIKSNPLT
KDIVIKMIPNVSDMSQCTGSVMENYKTRLNGILTPIKGALEIYKNNTHDL
VGDVRLAGVIMAGVAIGIATAAQITAGVALYEAMKNADNINKLKSSIEST
NEAVVKLCETAEKCVYVLTALQDYINTNLVPTIDKISCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
DSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVIISLGKYLGSVNYNSEGIAIGPPVFTDKV
DISSQISSMNQSLQQSKDYIKEAQRLLDTVNPSLklmkqiedkieeilsk
iyhieneiarikkligeapgglvprgshhhhhhsawshpqfek

NiV16
(SEQ ID NO: 16)
mysmqlascvtltlvllvnsQGILHYEKLSKIGLVKGVTRKYKIKSNPLT
KDIVIKMIPNVSNMSQCTGSVMENYKTRLNGILTPIKGALEIYKNNTHDL
VGDVRLAGVIMAGVAIGIATAAQITAGVALYEAMKNADNINKLKSSIEST
NEAVVKLQETAEKTVYVLTALQDYINTNLVPTIDKISCKQTELSLDLALS
KYLSDLLFVFGPNLQDPVSNSMTFQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVIISLGKYLGSVNYNSEGIAIGPPVFTDKV
DISSQISSMNQSLQQSKDYIKEAQRLLDTVNPSLklmkqiedkieeilsk
iyhieneiarikkligeapgglvprgshhhhhhsawshpqfek

NiVop1
(SEQ ID NO: 17)
mysmqlascvtltlvllvnsQGILHYEKLSKIGLVKGVTRKYKIKSNPLT
KDIVIKMIPNVSNMSQCTGSVMENYKTRLNGILTPIKGALEIYKNNTHDL
VGDVRLAGVCMAGVAIGIATAAQITAGVALYEAMKNADNINKLKSSIEST
NEAVVKLQETAEKTVYVFTALQDYINTNLVPTIDKISCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVcISLGKYLGSVNYNSEGIAIGPPVFTDKV
DISSQISSMNQSLQQSKDYIKEAQRLLDTVNPSLKLMKQIEDKIEEILSK
IYHIENEIARIKKLIGEAPGGLVPRGSHHHHHHSAWSHPQFEK

NiVop2
(SEQ ID NO: 18)
mysmqlascvtltlvllvnsQGILHYEKLSKIGLVKGVTRKYKIKSNPLT
KDIVIKMIPNVSNMSQCTGSVMENYKTRLNGILTPIKGALEIYKNNTHDC
VGDVRLAGVcMAGVAIGIATAAQITAGVALYEAMKNADNINKLKSSIEST
NEAVVKLQETAEKTVYVFTALQDYINTNLVPTIDKISCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVIISLGKYLGSVNYNSEGIAIGPPVFTDKV
DISSQISSMNQSLQQSKDYIKEAQRLLDTVNPSLKLMKQIEDKIEEILSK
IYHIENEIARIKKLIGEAPGGLVPRGSHHHHHHSAWSHPQFEK

NiVop3
(SEQ ID NO: 19)
mysmqlascvtltlvllvnsQGILHYEKLSKIGLVKGVTRKYKIKSNPLT
KDIVIKMIPNVSNMSQCTGSVMENYKTRLNGILTPIKGALETYKNNTgsg
IMAGVAIGIATAAQITAGVALYEAMKNADNINKLKSSIESTNEAVVKLQE
TAEKTVYVFTALQDYINTNLVPTIDKISCKQTELSLDLALSKYLSDLLFV
FGPNLQDPVSNSMTIQAISQAFGGNYETLLRTLGYATEDFDDLLESDSIT
GQIIYVDLSSYYIIVRVYFPILTEIQQAYIQELLPVSFNNDNSEWISIVP
NFILVRNTLISNIEIGFCLITKRSVICNQDYATPMTNNMRECLTGSTEKC
PRELVVSSHVPRFALSNGVLFANCISVTCQCQTTGRAISQSGEQTLLMID
NTTCPTAVLGNVIISLGKYLGSVNYNSEGIAIGPPVFTDKVDISSQISSM
NQSLQQSKDYIKEAQRLLDTVNPSLKLMKQIEDKIEEILSKIYHIENEIA
RIKKLIGEAPGGLVPRGSHHHHHHSAWSHPQFEK

NiVop4
(SEQ ID NO: 20)
mysmqlascvtltlvllvnsQGILHYEKLSKIGLVKGVTRKYKIKSNPLT
KDIVIKMIPNVSNMSQCTGSVMENYKTRLNGILTPIKGALEIYKNNTHDL
VGDVRLAGVcMAGVAIGIATAAQITAGVALYEAMKNADNINKLKSSIEST
NEAVVKLQETAEKTVYVLTALQDYINTNLVPTIDKIPCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVcISLGKYLGSVNYNSEGIAIGPPVFTDKV
DISSQISSMNQSLQQSKDYIKEAQRLLDTVNPSLKLMKQIEDKIEEILSK
IYHIENEIARIKKLIGEAPGGLVPRGSHHHHHHSAWSHPQFEK

NiVop5
(SEQ ID NO: 21)
mysmqlascvtltlvllvnsQGILHYEKLSKIGLVKGVTRKYKIKSNPLT
KDIVIKMIPNVSNMSQCTGSVMENYKTRLNGILTPIKGALEIYKNNTHDC
VGDVRLAGVcMAGVAIGIATAAQITAGVALYEAMKNADNINKLKSSIEST
NEAVVKLQETAEKTVYVLTALQDYINTNLVPTIDKIPCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVIISLGKYLGSVNYNSEGIAIGPPVFTDKV
DISSQISSMNQSLQQSKDYIKEAQRLLDTVNPSLKLMKQIEDKIEEILSK
IYHIENEIARIKKLIGEAPGGLVPRGSHHHHHHSAWSHPQFEK

NiVop6
(SEQ ID NO: 22)
mysmqlascvtltlvllvnsQGILHYEKLSKIGLVKGVTRKYKIKSNPLT
KDIVIKMIPNVSNMSQCTGSVMENYKTRLNGILTPIKGALEIYKNNTgsg
IMAGVAIGIATAAQITAGVALYEAMKNADNINKLKSSIESTNEAVVKLQE
TAEKTVYVLTALQDYINTNLVPTIDKIPCKQTELSLDLALSKYLSDLLFV
FGPNLQDPVSNSMTIQAISQAFGGNYETLLRTLGYATEDFDDLLESDSIT
GQIIYVDLSSYYIIVRVYFPILTEIQQAYIQELLPVSFNNDNSEWISIVP
NFILVRNTLISNIEIGFCLITKRSVICNQDYATPMTNNMRECLTGSTEKC
PRELVVSSHVPRFALSNGVLFANCISVTCQCQTTGRAISQSGEQTLLMID
NTTCPTAVLGNVIISLGKYLGSVNYNSEGIAIGPPVFTDKVDISSQISSM
NQSLQQSKDYIKEAQRLLDTVNPSLKLMKQIEDKIEEILSKIYHIENEIA
RIKKLIGEAPGGLVPRGSHHHHHHSAWSHPQFEK

NiVop7
(SEQ ID NO: 23)
mysmqlascvtltlvllvnsQGILHYEKLSKIGLVKGVTRKYKIKSNPLT
KDIVIKMIPNVSNMSQCTGSVMENYKTRLNGILTPIKGALEIYKNNTHDL
VGDVRLAGVcMAGVAIGIATAAQITAGVALYEAMKNADNINKLKSSIEST
NEAVVKLQETAEKTVYVFTALQDYINTNLVPTIDKIPCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVcISLGKYLGSVNYNSEGIAIGPPVFTDKV
DISSQISSMNQSLQQSKDYIKEAQRLLDTVNPSLKLMKQIEDKIEEILSK
IYHIENEIARIKKLIGEAPGGLVPRGSHHHHHHSAWSHPQFEK

NiVop8
(SEQ ID NO: 24)
mysmqlascvtltlvllvnsQGILHYEKLSKIGLVKGVTRKYKIKSNPLT
KDIVIKMIPNVSNMSQCTGSVMENYKTRLNGILTPIKGALEIYKNNTHDc
VGDVRLAGVcMAGVAIGIATAAQITAGVALYEAMKNADNINKLKSSIEST
NEAVVKLQETAEKTVYVFTALQDYINTNLVPTIDKIPCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVIISLGKYLGSVNYNSEGIAIGPPVFTDKV
DISSQISSMNQSLQQSKDYIKEAQRLLDTVNPSLKLMKQIEDKIEEILSK
IYHIENEIARIKKLIGEAPGGLVPRGSHHHHHHSAWSHPQFEK

NiVop9
(SEQ ID NO: 25)
mysmqlascvtltlvllvnsQGILHYEKLSKIGLVKGVTRKYKIKSNPLT
KDIVIKMIPNVSNMSQCTGSVMENYKTRLNGILTPIKGALETYKNNTgsg
IMAGVAIGIATAAQITAGVALYEAMKNADNINKLKSSIESTNEAVVKLQE
TAEKTVYVFTALQDYINTNLVPTIDKIPCKQTELSLDLALSKYLSDLLFV
FGPNLQDPVSNSMTIQAISQAFGGNYETLLRTLGYATEDFDDLLESDSIT
GQIIYVDLSSYYIIVRVYFPILTEIQQAYIQELLPVSFNNDNSEWISIVP
NFILVRNTLISNIEIGFCLITKRSVICNQDYATPMTNNMRECLTGSTEKC
PRELVVSSHVPRFALSNGVLFANCISVTCQCQTTGRAISQSGEQTLLMID
NTTCPTAVLGNVIISLGKYLGSVNYNSEGIAIGPPVFTDKVDISSQISSM
NQSLQQSKDYIKEAQRLLDTVNPSLKLMKQIEDKIEEILSKIYHIENEIA
RIKKLIGEAPGGLVPRGSHHHHHHSAWSHPQFEK

NiVop12
(SEQ ID NO: 26)
mysmqlascvtltlvllvnsQGILHYEKLSKIGLVKGVTRKYKIKSNPLT
KDIVIKMIPNVSNMSQCTGSVMENYKTRLNGILTPIKGALEIYKNNTHDL
VGDVRLAGVIMAGVAIGIATAAQITAGVALYEAMKNADNINKLKSSIEST
NEAVVKLQETAEKTVYVFTALQDYINTNLVPTIDKIpCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVIISLGKYLGSVNYNSEGIAIGPPVFTDKV
DISSQISSMNQSLQQSKDYIKEAQRLLDTVNPSLKLMKQIEDKIEEILSK
IYHIENEIARIKKLIGEAPGGLVPRGSHHHHHHSAWSHPQFEK

NiVop13
(SEQ ID NO: 27)
mysmqlascvtltlvllvnsQGILHYEKLSKIGLVKGVTRKYKIKSNPLT
KDIVIKMIPNVSNMSGCTGSVMENYKTRLNGILTPIKGALEIYKNNTHDL
VGDVRLAGVIMAGVAIGIATAAQITAGVALYEAMKNADNINKLKSSIEST
NEAVVKLQETAEKTVYVFTALQDYINTNLVPTIDKIpCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVIISLGKYLGSVNYNSEGIAIGPPVFTDKV
DISSQISSMNQSLQQSKDYIKEAQRLLDTVNPSLKLMKQIEDKIEEILSK
IYHIENEIARIKKLIGEAPGGLVPRGSHHHHHHSAWSHPQFEK

NiVop14
(SEQ ID NO: 28)
mysmqlascvtltlvllvnsQGILHYEKLSKIGLVKGVTRKYKIKSNPLT
KDIVIKMIPNVSNMSGCTGSVMENYKTRLNGILTPIKGALEIYKNNTHDC
VGDVRLAGVcMAGVAIGIATAAQITAGVALYEAMKNADNINKLKSSIEST
NEAVVKLQETAEKTVYVFTALQDYINTNLVPTIDKIPCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVIISLGKYLGSVNYNSEGIAIGPPVFTDKV
DISSQISSMNQSLQQSKDYIKEAQRLLDTVNPSLKLMKQIEDKIEEILSK
IYHIENEIARIKKLIGEAPGGLVPRGSHHHHHHSAWSHPQFEK

NiVop15
(SEQ ID NO: 29)
mysmqlascvtltlvllvnsQGILHYEKLSKIGLVKGVTRKYKIKSNPLT
KDIVIKMIPNVSNMSGCTGSVMENYKTRLNGILTPIKGALEIYKNNTHDc
VGDVRLAGVcMAGVAIGIATAAQITAGVALYEAMKNADNINKLKSSIEST
NEAVVKLQETAEKTVYVFTALQDYINTNLVPTIDKISCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVIISLGKYLGSVNYNSEGIAIGPPVFTDKV
DISSQISSMNQSLQQSKDYIKEAQRLLDTVNPSLKLMKQIEDKIEEILSK
IYHIENEIARIKKLIGEAPGGLVPRGSHHHHHHSAWSHPQFEK

NiVop16
(SEQ ID NO: 30)
mysmqlascvtltlvllvnsQGILHYEKLSKIGLVKGVTRKYKIKSNPLT
KDIVIKMIPNVSNMSGCTGSVMENYKTRLNGILTPIKGALEIYKNNTHDc
VGDVRLAGVcMAGVAIGIATAAQITAGVALYEAMKNADNINKLKSSIEST
NEAVVKLQETAEKTVYVLTALQDYINTNLVPTIDKISCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVIISLGKYLGSVNYNSEGIAIGPPVFTDKV
DISSQISSMNQSLQQSKDYIKEAQRLLDTVNPSLKLMKQIEDKIEEILSK
IYHIENEIARIKKLIGEAPGGLVPRGSHHHHHHSAWSHPQFEK

NiVop17
(SEQ ID NO: 31)
mysmqlascvtltlvllvnsQGILHYEKLSKIGLVKGVTRKYKIKSNPLT
KDIVIKMIPNVSNMSQCTGSVMENYKTRLNGILTPIKGALEIYKNNTHDc
VGDVRLAGVcMAGVAIGIATAAQITAGVALYEAMKNADNINKLKSSIEST
NEAVVKLCETAEKCVYVFTALQDYINTNLVPTIDKIPCKQTELSLDLALS
KYLSDLLFVFGPNLQDPVSNSMTIQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVIISLGKYLGSVNYNSEGIAIGPPVFTDKV
DISSQISSMNQSLQQSKDYIKEAQRLLDTVNPSLKLMKQIEDKIEEILSK
IYHIENEIARIKKLIGEAPGGLVPRGSHHHHHHSAWSHPQFEK

NiVop18
(SEQ ID NO: 32)
mysmqlascvtltlvllvnsQGILHYEKLSKIGLVKGVTRKYKIKSNPLT
KDIVIKMIPNVSNMSQCTGSVMENYKTRLNGILTPIKGALEIYKNNTHDc
VGDVRLAGVcMAGVAIGIATAAQITCGVALYEAMKNADNINKLKSSIEST
NEAVVKLQETAEKTVYVFTALQDYINTNLVPTIDKIPCKQTELSLDLALS
KYLSDLLFVFGPNLQDPCSNSMTIQAISQAFGGNYETLLRTLGYATEDFD
DLLESDSITGQIIYVDLSSYYIIVRVYFPILTEIQQAYIQELLPVSFNND
NSEWISIVPNFILVRNTLISNIEIGFCLITKRSVICNQDYATPMTNNMRE
CLTGSTEKCPRELVVSSHVPRFALSNGVLFANCISVTCQCQTTGRAISQS
GEQTLLMIDNTTCPTAVLGNVIISLGKYLGSVNYNSEGIAIGPPVFTDKV
DISSQISSMNQSLQQSKDYIKEAQRLLDTVNPSLKLMKQIEDKIEEILSK
IYHIENEIARIKKLIGEAPGGLVPRGSHHHHHHSAWSHPQFEK

The above sequences include an N-terminal signal peptide, a NiV F ectodomain, a GCN4 trimerization domain, a thrombin cleavage site, a HIS tag and a Strep tag, as well as various linker residues between segments.

### Example 2

### NiV G Multimers

The example illustrates embodiments of immunogens including multimers of the NiV G ectodomain.

The N-terminus of the NiV G ectodomain was linked to a T4 fibritin trimerization domain, and a C-terminal his tag. Different versions of the construct including one, two, or three G ectodomains in series were designed. A further multimer was designed that included two G ectodomains, one on either end (N- and C-termini) of the T4 fibritin trimerization domain (Fd). Sequences are as follows:

Fd-G
(SEQ ID NO: 34)
mysmqlascvtltlvllvnsQGSGYIPEAPRDGQAYVRKDGEWVLLSTFL
GSGGGGGgvsnlvglpnniclqktsnqilkpklisytlpvvgqsgtcitd
pllamdegyfayshlerigscsrgvskqriigvgevldrgdevpslfmtn
vwtppnpntvyhcsavynnefyyvlcavstvgdpilnstywsgslmmtrl
avkpksngggynqhqlalrsiekgrydkvmpygpsgikqgdtlyfpavgf
lvrtefkyndsncpitkcqyskpencrlsmgirpnshyilrsgllkynls
dgenpkvvfieisdqrlsigspskiydslgqpvfyqasfswdtmikfgdv
ltvnplvvnwrnntvisrpgqsqcprfntcpeicwegvyndaflidrinw
isagvfldsnqtaenpvftvfkdneilyraqlasedtnaqktitncfllk
nkiwcislveiydtgdnvirpklfavkipeqctgglvprgshhhhhhsaw
shpqfek

Fd-GG
(SEQ ID NO: 35)
mysmqlascvtltlvllvnsQGSGYIPEAPRDGQAYVRKDGEWVLLSTFL
GSGGGGGgvsnlvglpnniclqktsnqilkpklisytlpvvgqsgtcitd
pllamdegyfayshlerigscsrgvskqriigvgevldrgdevpslfmtn
vwtppnpntvyhcsavynnefyyvlcavstvgdpilnstywsgslmmtrl
avkpksngggynqhqlalrsiekgrydkvmpygpsgikqgdtlyfpavgf
lvrtefkyndsncpitkcqyskpencrlsmgirpnshyilrsgllkynls
dgenpkvvfieisdqrlsigspskiydslgqpvfyqasfswdtmikfgdv
ltvnplvvnwrnntvisrpgqsqcprfntcpeicwegvyndaflidrinw
isagvfldsnqtaenpvftvfkdneilyraqlasedtnaqktitncfllk
nkiwcislveiydtgdnvirpklfavkipeqctggGGGGgvsnlvglpnn
iclqktsnqilkpklisytlpvvgqsgtcitdpllamdegyfayshleri
gscsrgvskqriigvgevldrgdevpslfmtnvwtppnpntvyhcsavyn
nefyyvlcavstvgdpilnstywsgslmmtrlavkpksngggynqhqlal
rsiekgrydkvmpygpsgikqgdtlyfpavgflvrtefkyndsncpitkc
qyskpencrlsmgirpnshyilrsgllkynlsdgenpkvvfieisdqrls
igspskiydslgqpvfyqasfswdtmikfgdvltvnplvvnwrnntvisr
pgqsqcprfntcpeicwegvyndaflidrinwisagvfldsnqtaenpvf
tvfkdneilyraqlasedtnaqktitncfllknkiwcislveiydtgdnv
irpklfavkipeqctgglvprgshhhhhhsawshpqfek

Fd-GGG
(SEQ ID NO: 36)
mysmqlascvtltlvllvnsQGSGYIPEAPRDGQAYVRKDGEWVLLSTFL
GSGGGGGgvsnlvglpnniclqktsnqilkpklisytlpvvgqsgtcitd
pllamdegyfayshlerigscsrgvskqriigvgevldrgdevpslfmtn
vwtppnpntvyhcsavynnefyyvlcavstvgdpilnstywsgslmmtrl
avkpksngggynqhqlalrsiekgrydkvmpygpsgikqgdtlyfpavgf
lvrtefkyndsncpitkcqyskpencrlsmgirpnshyilrsgllkynls
dgenpkvvfieisdqrlsigspskiydslgqpvfyqasfswdtmikfgdv
ltvnplvvnwrnntvisrpgqsqcprfntcpeicwegvyndaflidrinw
isagvfldsnqtaenpvftvfkdneilyraqlasedtnaqktitncfllk
nkiwcislveiydtgdnvirpklfavkipeqctggGGGGgvsnlvglpnn
iclqktsnqilkpklisytlpvvgqsgtcitdpllamdegyfayshleri
gscsrgvskqriigvgevldrgdevpslfmtnvwtppnpntvyhcsavyn
nefyyvlcavstvgdpilnstywsgslmmtrlavkpksngggynqhqlal
rsiekgrydkvmpygpsgikqgdtlyfpavgflvrtefkyndsncpitkc
qyskpencrlsmgirpnshyilrsgllkynlsdgenpkvvfieisdqrls
igspskiydslgqpvfyqasfswdtmikfgdvltvnplvvnwrnntvisr
pgqsqcprfntcpeicwegvyndaflidrinwisagvfldsnqtaenpvf
tvfkdneilyraqlasedtnaqktitncfllknkiwcislveiydtgdnv
irpklfavkipeqctggGGGGgvsnlvglpnniclqktsnqilkpklisy
tlpvvgqsgtcitdpllamdegyfayshlerigscsrgvskqriigvgev
ldrgdevpslfmtnvwtppnpntvyhcsavynnefyyvlcavstvgdpil
nstywsgslmmtrlavkpksngggynqhqlalrsiekgrydkvmpygpsg
ikqgdtlyfpavgflvrtefkyndsncpitkcqyskpencrlsmgirpns
hyilrsgllkynlsdgenpkvvfieisdqrlsigspskiydslgqpvfyq
asfswdtmikfgdvltvnplvvnwrnntvisrpgqsqcprfntcpeicwe
gvyndaflidrinwisagvfldsnqtaenpvftvfkdneilyraqlased
tnaqktitncfllknkiwcislveiydtgdnvirpklfavkipeqctggl
vprgshhhhhhsawshpqfek

G-Fd-G
(SEQ ID NO: 37)
mysmqlascvtltlvllvnsQEgvsnlvglpnniclqktsnqilkpklis
ytlpvvgqsgtcitdpllamdegyfayshlerigscsrgvskqriigvge
vldrgdevpslfmtnvwtppnpntvyhcsavynnefyyvlcavstvgdpi
lnstywsgslmmtrlavkpksngggynqhqlalrsiekgrydkvmpygps
gikqgdtlyfpavgflvrtefkyndsncpitkcqyskpencrlsmgirpn
shyilrsgllkynlsdgenpkvvfieisdqrlsigspskiydslgqpvfy
qasfswdtmikfgdvltvnplvvnwrnntvisrpgqsqcprfntcpeicw
egvyndaflidrinwisagvfldsnqtaenpvftvfkdneilyraqlase
dtnaqktitncfllknkiwcislveiydtgdnvirpklfavkipeqctgg
GSGYIPEAPRDGQAYVRKDGEWVLLSTFLGSGGGGGgvsnlvglpnnicl
qktsnqilkpklisytlpvvgqsgtcitdpllamdegyfayshlerigsc
srgvskqriigvgevldrgdevpslfmtnvwtppnpntvyhcsavynnef
yyvlcavstvgdpilnstywsgslmmtrlavkpksngggynqhqlalrsi
ekgrydkvmpygpsgikqgdtlyfpavgflvrtefkyndsncpitkcqys
kpencrlsmgirpnshyilrsgllkynlsdgenpkvvfieisdqrlsigs
pskiydslgqpvfyqasfswdtmikfgdvltvnplvvnwrnntvisrpgq
sqcprfntcpeicwegvyndaflidrinwisagvfldsnqtaenpvftvf
kdneilyraqlasedtnaqktitncfllknkiwcislveiydtgdnvirp
klfavkipeqctgglvprgshhhhhhsawshpqfek

The above sequences include one or more G ectodomains, an N-terminal signal peptide, HIS tag, Strep tag, and a thrombin cleavage site to remove the two tags, a trimerization domain, and various linker residues between segments.

The Fd-G, Fd-GG, Fd-GGG, and G-Fd-G were expressed in cells and purified as discussed above for soluble NiV F ectodomain trimers in Example 1. Each of the constructs was successfully purified. The expression levels of the Fd-G and G-Fd-G constructs were 8 and 2.3 mg/mL.

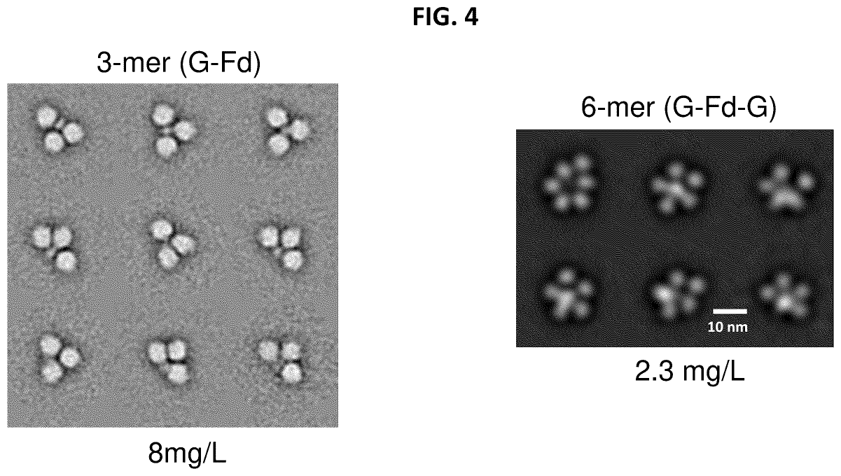

Negative stain EM showed that all of these constructs formed multimers. Exemplary negative stain EM images for G-Fd and G-Fd-G multimers are shown in FIG. 4.

Additional NiV G ectodomain multimers were constructed with the G ectodomain linked to the N-terminus of a subunit of a self-assembling ferritin nanoparticle. Different versions of the construct including a glycine-serine linker of 5, 15, 25, or 35 residues between the G ectodomain and the ferritin subunit. A further multimer was designed that included a NiV G ectodomain linked to the N-terminus of a subunit of a self-assembling lumazine synthase nanoparticle. Different versions of the constructs including a glycine-serine linker of 5, 15, 25, or 35 residues between the G ectodomain and the ferritin or lumazine synthase subunit were generated. Sequences are as follows:

G-ln5-Fer
(SEQ ID NO: 38)
mysmqlascvtltlvllvnsqHHHHHHGSAWSHPQFEKGGLVPRGSGnsq
rpqtegvsnlvglpnniclqktsnqilkpklisytlpvvgqsgtcitdpl
lamdegyfayshlerigscsrgvskqriigvgevldrgdevpslfmtnvw
tppnpntvyhcsavynnefyyvlcavstvqdpilnstywsqslmmtrlav
kpksnqqqynqhqlalrsiekqrydkvmpyqpsqikqqdtlyfpavgflv
rtefkyndsncpitkcqyskpencrlsmgirpnshyilrsgllkynlsdg
enpkvvfieisdqrlsigspskiydslgqpvfyqasfswdtmikfgdvlt
vnplvvnwrnntvisrpgqsqcprfntcpeicwegvyndaflidrinwis
agvfldsnqtaenpvftvfkdneilyraqlasedtnaqktitncfllknk
iwcislveiydtgdnvirpklfavkipeqctgggSGGDIIKLLNEQVNKE
MQSSNLYMSMSSWCYTHSLDGAGLFLFDHAAEEYEHAKKLIIFLNENNVP
VQLTSISAPEHKFEGLTQIFQKAYEHEQHISESINNIVDHAIKSKDHATF
NFLQWYVAEQHEEEVLFKDILDKIELIGNENHGLYLADQYVKGTAKSRKS
GS

G-ln15-Fer
(SEQ ID NO: 39)
mysmqlascvtltlvllvnsqHHHHHHGSAWSHPQFEKGGLVPRGSGnsq
rpqtegvsnlvglpnniclqktsnqilkpklisytlpvvgqsgtcitdpl
lamdegyfayshlerigscsrgvskqriigvgevldrgdevpslfmtnvw
tppnpntvyhcsavynnefyyvlcavstvgdpilnstywsgslmmtrlav
kpksngggynqhqlalrsiekgrydkvmpygpsgikqgdtlyfpavgflv
rtefkyndsncpitkcqyskpencrlsmgirpnshyilrsgllkynlsdg
enpkvvfieisdqrlsigspskiydslgqpvfyqasfswdtmikfgdvlt
vnplvvnwrnntvisrpgqsqcprfntcpeicwegvyndaflidrinwis
agvfldsnqtaenpvftvfkdneilyraqlasedtnaqktitncfllknk
iwcislveiydtgdnvirpklfavkipeqctgggSGGggsggSGGgDIIK
LLNEQVNKEMQSSNLYMSMSSWCYTHSLDGAGLFLFDHAAEEYEHAKKLI
IFLNENNVPVQLTSISAPEHKFEGLTQIFQKAYEHEQHISESINNIVDHA
IKSKDHATFNFLQWYVAEQHEEEVLFKDILDKIELIGNENHGLYLADQYV
KGIAKSRKSGS

G-ln25-Fer
(SEQ ID NO: 40)
mysmqlascvtltlvllvnsqHHHHHHGSAWSHPQFEKGGLVPRGSGns
qrpqtegvsnlvglpnniclqktsnqilkpklisytlpvvgqsgtcitd
pllamdegyfayshlerigscsrgvskqriigvgevldrgdevpslfmt
nvwtppnpntvyhcsavynnefyyvlcavstvgdpilnstywsgslmmt
rlavkpksngggynqhqlalrsiekgrydkvmpygpsgikqgdtlyfpa
vgflvrtefkyndsncpitkcqyskpencrlsmgirpnshyilrsgllk
ynlsdgenpkvvfieisdqrlsigspskiydslgqpvfyqasfswdtmi
kfgdvltvnplvvnwrnntvisrpgqsqcprfntcpeicwegvyndafl
idrinwisagvfldsnqtaenpvftvfkdneilyraqlasedtnaqkti
tncfllknkiwcislveiydtgdnvirpklfavkipeqctgggSGGggs
ggSGGggSGGggSGGgDIIKLLNEQVNKEMQSSNLYMSMSSWCYTHSLD
GAGLFLFDHAAEEYEHAKKLIDKIELIGNENHGLYLADQYVKGIAKSRK
SGS

G-ln35-Fer
(SEQ ID NO: 41)
mysmqlascvtltlvllvnsqHHHHHHGSAWSHPQFEKGGLVPRGSGns
qrpqtegvsnlvglpnniclqktsnqilkpklisytlpvvgqsgtcitd
pllamdegyfayshlerigscsrgvskqriigvgevldrgdevpslfmt
nvwtppnpntvyhcsavynnefyyvlcavstvgdpilnstywsgslmmt
rlavkpksngggynqhqlalrsiekgrydkvmpygpsgikqgdtlyfpa
vgflvrtefkyndsncpitkcqyskpencrlsmgirpnshyilrsgllk
ynlsdgenpkvvfieisdqrlsigspskiydslgqpvfyqasfswdtmi
kfgdvltvnplvvnwrnntvisrpgqsqcprfntcpeicwegvyndafl
idrinwisagvfldsnqtaenpvftvfkdneilyraqlasedtnaqkti
tncfllknkiwcislveiydtgdnvirpklfavkipeqctgggSGGggs
ggSGGggSGGggSGGggSGGggSGGgDIIKLLNEQVNKEMQSSNLYMSM
SSWCYTHSLDGAGLFLFDHAAEEYEHAKKLIIFLNENNVPVQLTSISAP
EHKFEGLTQIFQKAYEHEQHISESINNIVDHAIKSKDHATFNFLQWYVA
EQHEEEVLFKDILDKIELIGNENHGLYLADQYVKGIAKSRKSGS

G-LS
(SEQ ID NO: 42)
mysmqlascvtltlvllvnsqHHHHHHGSAWSHPQFEKGGLVPRGSGns
qrpqtegvsnlvglpnniclqktsnqilkpklisytlpvvgqsgtcitd
pllamdegyfayshlerigscsrgvskqriigvgevldrgdevpslfmt
nvwtppnpntvyhcsavynnefyyvlcavstvgdpilnstywsgslmmt
rlavkpksngggynqhqlalrsiekgrydkvmpygpsgikqgdtlyfpa
vgflvrtefkyndsncpitkcqyskpencrlsmgirpnshyilrsgllk
ynlsdgenpkvvfieisdqrlsigspskiydslgqpvfyqasfswdtmi
kfgdvltvnplvvnwrnntvisrpgqsqcprfntcpeicwegvyndafl
idrinwisagvfldsnqtaenpvftvfkdneilyraqlasedtnaqkti
tncfllknkiwcislveiydtgdnvirpklfavkipeqctgggsgggsg
ggsMQIYEGKLTAEGLRFGIVASRFNHALVDRLVEGAIDCIVRHGGREE
DITLVRVPGSWEIPVAAGELARKEDIDAVIAIGVLIRGATPHFDYIASE
VSKGLADLSLELRKPITFGVITADTLEQAIERAGTKHGNKGWEAALSAI
EMANLFKSLR

The above sequences include one or more G ectodomains, self-assembling nanoparticle subunit, an N-terminal signal peptide, HIS tag, Strep tag, a thrombin cleavage site to remove the two tags, and various linker residues between segments.

The G-ln5-Fer, G-ln15-Fer, G-ln25-Fer, G-ln35-Fer, and G-LS constructs were expressed in cells and purified as discussed above for soluble NiV F ectodomain trimers in Example 1. Each of the constructs was successfully purified as a multimerized nanoparticle.

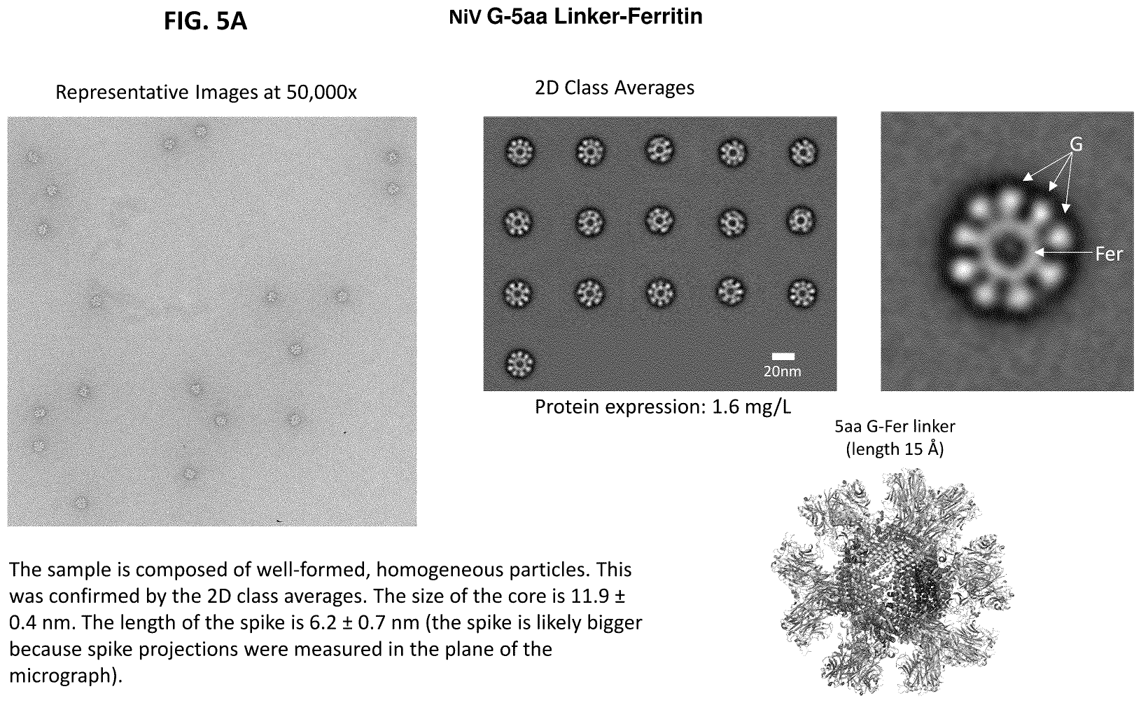

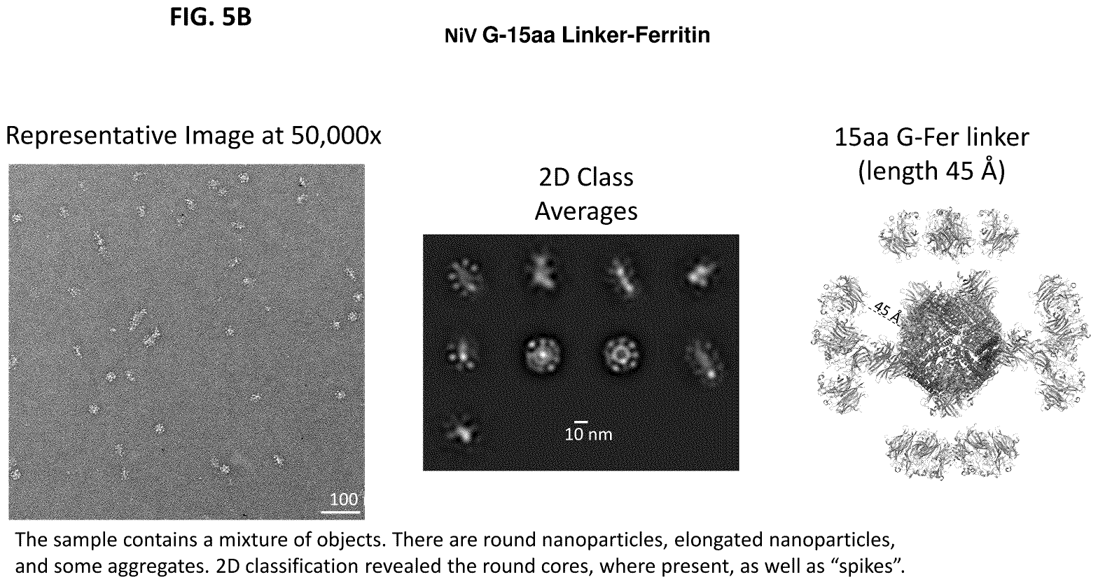

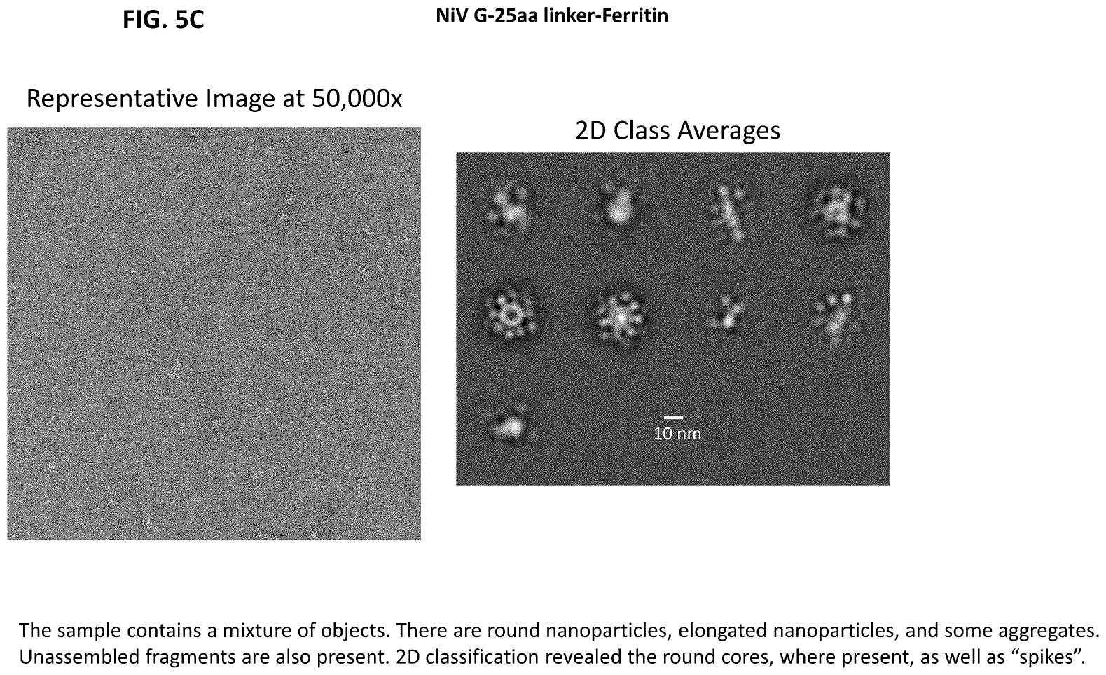

Negative stain EM showed that all of these constructs self-assembled into multimeric nanoparticles. Exemplary negative stain EM images for G-ln5-Fer, G-ln15-Fer, and G-ln25-Fer multimers are shown in FIGS. 5A-5C.

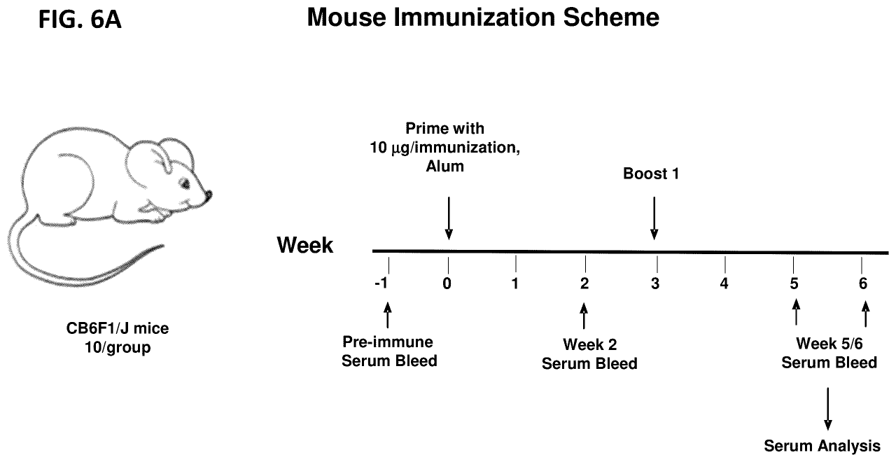

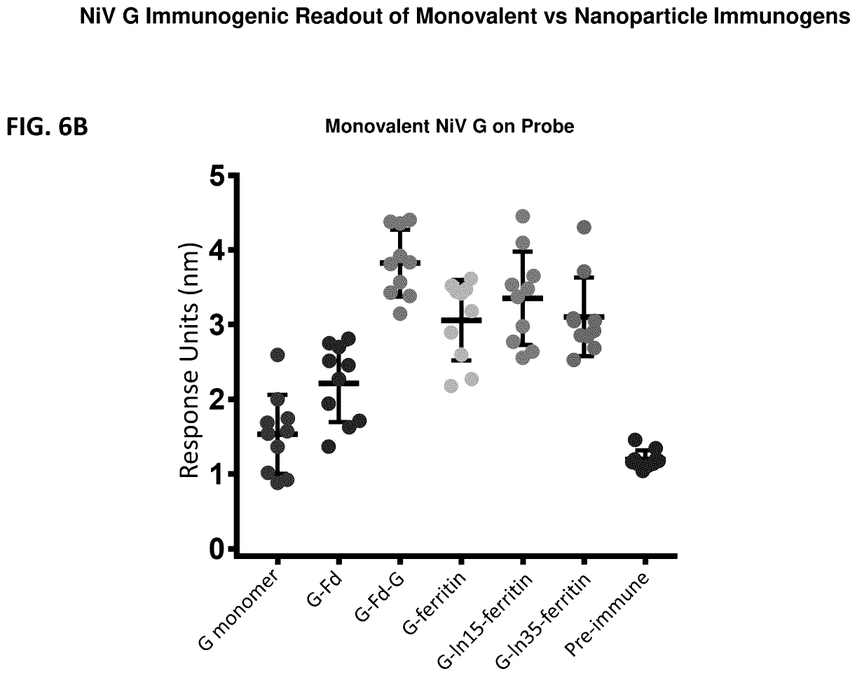

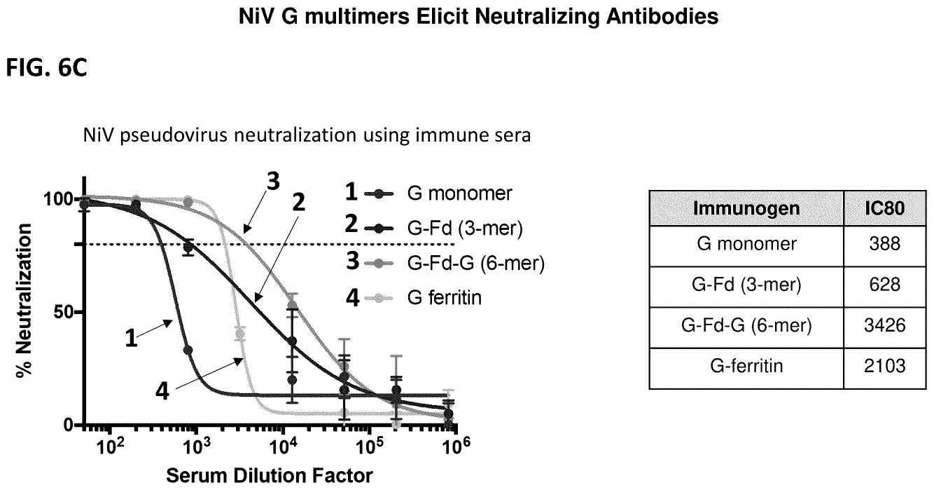

Immunization assays were conducted with the G-Fd, G-Fd-G, G-ln5-Fer, G-ln15-Fer, and G-ln35-Fer multimers. The assay was performed substantially as described in Example 1. CB6F1/J mice were immunized with 5 μg of multimer in Alum using the schedule shown in FIG. 6A. Sera from immunized mice was tested for binding to monomeric NiV G using an Octet binding assay (FIG. 6B). The NiV G was linked to the sensor and sera from the indicated immunizations assayed for binding. The immune sera was also assessed in a NiV pseudovirus neutralization assay (FIG. 6C), which showed that immune sera from animals treated with the multimeric NiV G constructs neutralized NiV. Thus, immunization with soluble multimeric NiV G constructs elicited a neutralizing immune response in an animal model.

### Example 3

### Multimers of NiV F-G Ectodomain Chimeras

The example illustrates embodiments of immunogens including multimers of the NiV F and G ectodomains.

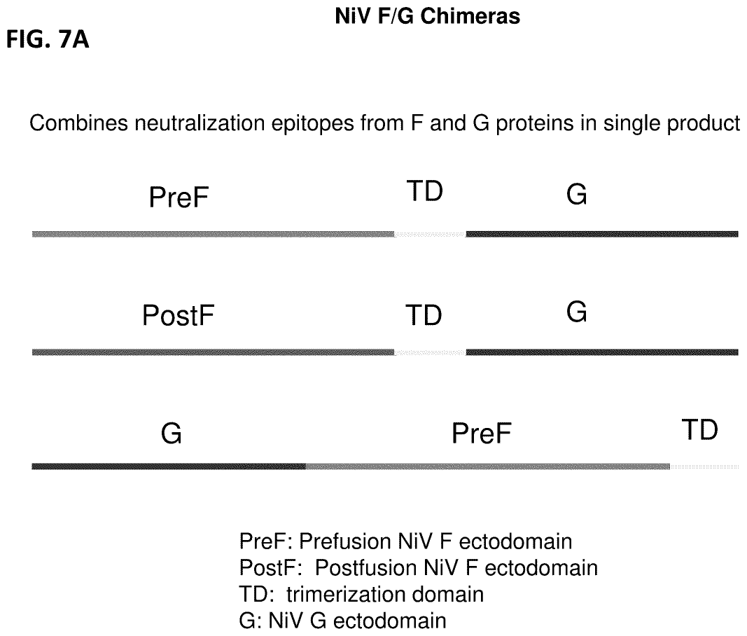

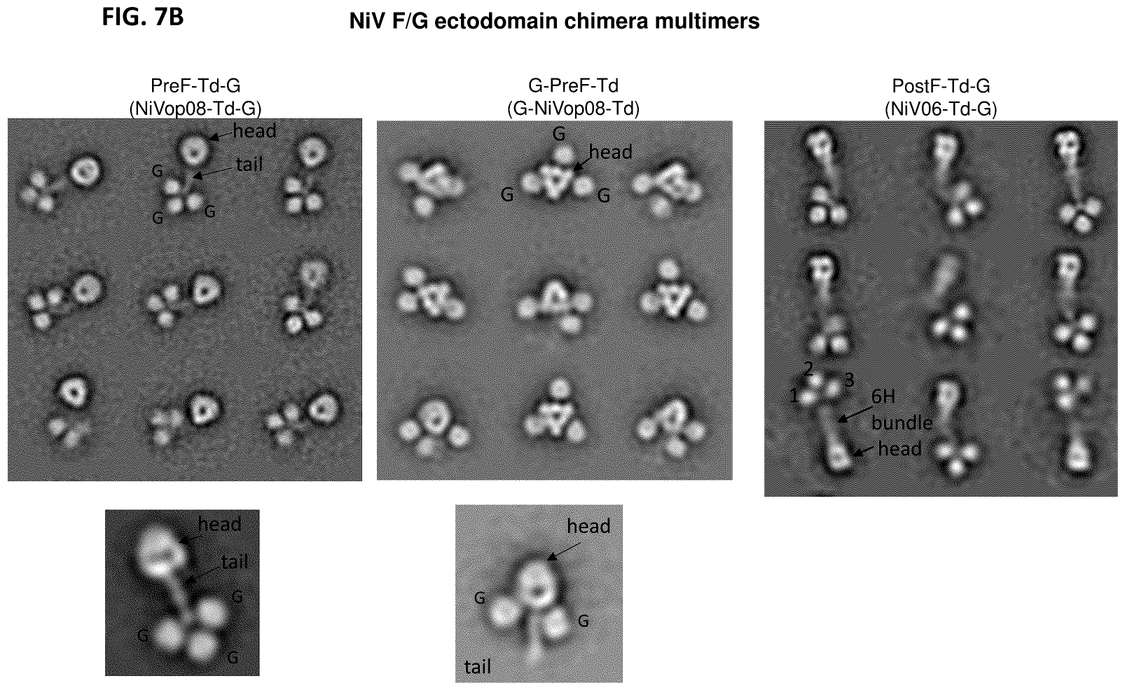

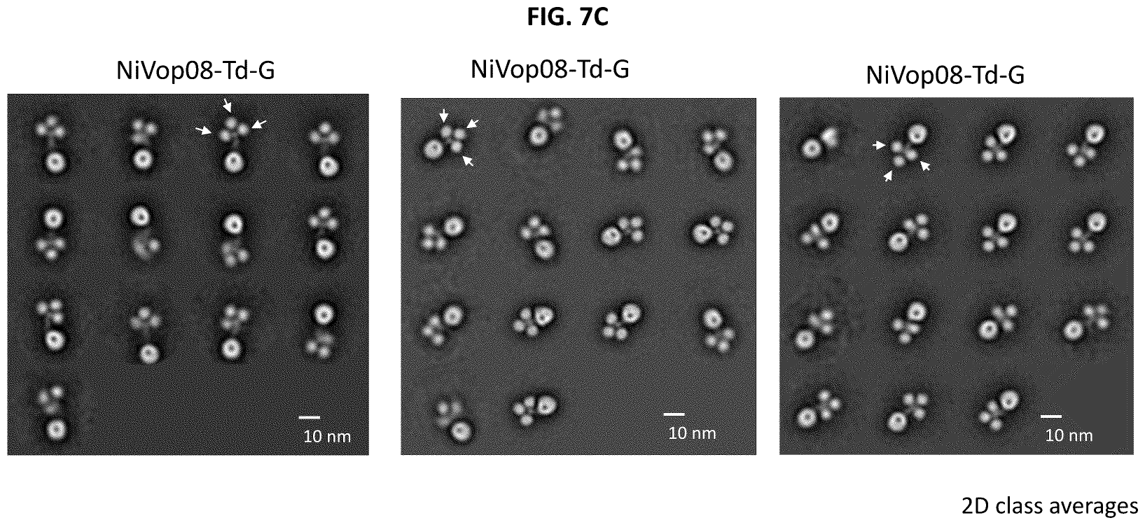

FIG. 7A illustrates the structure of the chimeric proteins included in the NiV F-G multimers. Multiple formats were assessed for the chimeric multimers, including:
preF-TD-G: prefusion F ectodomain (e.g., NiVop08) fused to C-terminal trimerization domain (e.g., GCN4, Fd, or GCN4 and Fd) fused to G ectodomain
G-preF-TD: G ectodomain fused to prefusion F ectodomain (e.g., NiVop08) fused to C-terminal trimerization domain (e.g., GCN4, Fd, or GCN4 and Fd)

As a control, a postfusion construct was also produced:
postF-TD-G: postfusion F ectodomain (e.g., NiV06) fused to fused to C-terminal trimerization domain (e.g., GCN4, Fd, or GCN4 and Fd) fused to G ectodomain
   Sequences are as follows:

NiVop08-TD(GCN4-Fd)-G
(SEQ ID NO: 43)
mysmqlascvtltlvllvnsQGILHYEKLSKIGLVKGVTRKYKIKSNPL
TKDIVIKMIPNVSNMSQCTGSVMENYKTRLNGILTPIKGALEIYKNNTH
DCVGDVRLAGVCMAGVAIGIATAAQITAGVALYEAMKNADNINKLKSSI
ESTNEAVVKLQETAEKTVYVFTALQDYINTNLVPTIDKIPCKQTELSLD
LALSKYLSDLLFVFGPNLQDPVSNSMTIQAISQAFGGNYETLLRTLGYA
TEDFDDLLESDSITGQIIYVDLSSYYIIVRVYFPILTEIQQAYIQELLP
VSFNNDNSEWISIVPNFILVRNTLISNIEIGFCLITKRSVICNQDYATP
MTNNMRECLTGSTEKCPRELVVSSHVPRFALSNGVLFANCISVTCQCQT
TGRAISQSGEQTLLMIDNTTCPTAVLGNVIISLGKYLGSVNYNSEGIAI
GPPVFTDKVDISSQISSMNQSLQQSKDYIKEAQRLLDTVNPSLKLMKQI
EDKIEEILSKIYHIENEIARIKKLIGEAPGSGYIPEAPRDGQAYVRKDG
EWVLLSTFLGSGGGGGgvsnlvglpnniclqktsnqilkpklisytlpv
vgqsgtcitdpllamdegyfayshlerigscsrgvskqriigvgevldr
gdevpslfmtnvwtppnpntvyhcsavynnefyyvlcavstvgdpilns
tywsgslmmtrlavkpksngggynqhqlalrsiekgrydkvmpygpsgi
kqgdtlyfpavgflvrtefkyndsncpitkcqyskpencrlsmgirpns
hyilrsgllkynlsdgenpkvvfieisdqrlsigspskiydslgqpvfy
qasfswdtmikfgdvltvnplvvnwrnntvisrpgqsqcprfntcpeic
wegvyndaflidrinwisagvfldsnqtaenpvftvfkdneilyraqla
sedtnaqktitncfllknkiwcislveiydtgdnvirpklfavkipeqc
tgglvprgshhhhhhsawshpqfek

G-NiVop08-TD (GCN4-Fd)
(SEQ ID NO: 44)
Mysmqlascvtltlvllvnsqrpqtegvsnlvglpnniclqktsnqilk
pklisytlpvvgqsgtcitdpllamdegyfayshlerigscsrgvskqr
iigvgevldrgdevpslfmtnvwtppnpntvyhcsavynnefyyvlcav
stvgdpilnstywsgslmmtrlavkpksngggynqhqlalrsiekgryd
kvmpygpsgikqgdtlyfpavgflvrtefkyndsncpitkcqyskpenc
rlsmgirpnshyilrsgllkynlsdgenpkvvfieisdqrlsigspski
ydslgqpvfyqasfswdtmikfgdvltvnplvvnwrnntvisrpgqsqc
prfntcpeicwegvyndaflidrinwisagvfldsnqtaenpvftvfkd
neilyraqlasedtnaqktitncfllknkiwcislveiydtgdnvirpk
lfavkipeqctgggQGILHYEKLSKIGLVKGVTRKYKIKSNPLTKDIVI
KMIPNVSNMSQCTGSVMENYKTRLNGILTPIKGALEIYKNNTHDCVGDV
RLAGVCMAGVAIGIATAAQITAGVALYEAMKNADNINKLKSSIESTNEA
VVKLQETAEKTVYVFTALQDYINTNLVPTIDKIPCKQTELSLDLALSKY
LSDLLFVFGPNLQDPVSNSMTIQAISQAFGGNYETLLRTLGYATEDFDD
LLESDSITGQIIYVDLSSYYIIVRVYFPILTEIQQAYIQELLPVSFNND
NSEWISIVPNFILVRNTLISNIEIGFCLITKRSVICNQDYATPMTNNMR
ECLTGSTEKCPRELVVSSHVPRFALSNGVLFANCISVTCQCQTTGRAIS
QSGEQTLLMIDNTTCPTAVLGNVIISLGKYLGSVNYNSEGIAIGPPVFT
DKVDISSQISSMNQSLQQSKDYIKEAQRLLDTVNPSLKLMKQIEDKIEE
ILSKIYHIENEIARIKKLIGEAPGSGYIPEAPRDGQAYVRKDGEWVLLS
TFLGSLVPRGSHHHHHHSAWSHPQFEK

NiV06-TD(GCN4-Fd)-G
(SEQ ID NO: 45)
MysmqlascvtltlvllvnsQGILHYEKLSKIGLVKGVTRKYKIKSNPL
TKDIVIKMIPNVSNMSQCTGSVMENYKTRLNGILTPIKGALEIYKNGGS
GVAIGIATAAQITAGVALYEAMKNADNINKLKSSIESTNEAVVKLQETA
EKTVYVLTALQDYINTNLVPTIDKISCKQTELSLDLALSKYLSDLLFVF
GPNLQDPVSNSMTIQAISQAFGGNYETLLRTLGYATEDFDDLLESDSIT
GQIIYVDLSSYYIIVRVYFPILTEIQQAYIQELLPVSFNNDNSEWISIV
PNFILVRNTLISNIEIGFCLITKRSVTAVLGNVIISLGKYLGSVNYNSE
GIAIGPPVFTDKVDISSQISSMNQSLQQSKDYIKEAQRLLDTVNPSLKL
MKQIEDKIEEILSKIYHIENEIARIKKLIGEAPGSGYIPEAPRDGQAYV
RKDGEWVLLSTFLGSGGGGGgvsnlvglpnniclqktsnqilkpklisy
tlpvvgqsgtcitdpllamdegyfayshlerigscsrgvskqriigvge
vldrgdevpslfmtnvwtppnpntvyhcsavynnefyyvlcavstvgdp
ilnstywsgslmmtrlavkpksngggynqhqlalrsiekgrydkvmpyg
psgikqgdtlyfpavgflvrtefkyndsncpitkcqyskpencrlsmgi
rpnshyilrsgllkynlsdgenpkvvfieisdqrlsigspskiydslgq
pvfyqasfswdtmikfgdvltvnplvvnwrnntvisrpgqsqcprfntc
peicwegvyndaflidrinwisagvfldsnqtaenpvftvfkdneilyr
aqlasedtnaqktitncfllknkiwcislveiydtgdnvirpklfavki
peqctgglvprgshhhhhhsawshpqfek

NiV06-TD(GCN4-Fd)-GG
(SEQ ID NO: 46)
MysmqlascvtltlvllvnsQGILHYEKLSKIGLVKGVTRKYKIKSNPL
TKDIVIKMIPNVSNMSQCTGSVMENYKTRLNGILTPIKGALEIYKNGGS
GVAIGIATAAQITAGVALYEAMKNADNINKLKSSIESTNEAVVKLQETA
EKTVYVLTALQDYINTNLVPTIDKISCKQTELSLDLALSKYLSDLLFVF
GPNLQDPVSNSMTIQAISQAFGGNYETLLRTLGYATEDFDDLLESDSIT
GQIIYVDLSSYYIIVRVYFPILTEIQQAYIQELLPVSFNNDNSEWISIV
PNFILVRNTLISNIEIGFCLITKRSVICNQDYATPMTNNMRECLTGSTE
KCPRELVVSSHVPRFALSNGVLFANCISVTCQCQTTGRAISQSGEQTLL
MIDNTTCPTAVLGNVIISLGKYLGSVNYNSEGIAIGPPVFTDKVDISSQ
ISSMNQSLQQSKDYIKEAQRLLDTVNPSLKLMKQIEDKIEEILSKIYHI
ENEIARIKKLIGEAPGSGYIPEAPRDGQAYVRKDGEWVLLSTFLGSGGG
GGgvsnlvglpnniclqktsnqilkpklisytlpvvgqsgtcitdplla
mdegyfayshlerigscsrgvskqriigvgevldrgdevpslfmtnvwt
ppnpntvyhcsavynnefyyvlcavstvgdpilnstywsgslmmtrlav
kpksngggynqhqlalrsiekgrydkvmpygpsgikqgdtlyfpavgfl
vrtefkyndsncpitkcqyskpencrlsmgirpnshyilrsgllkynls
dgenpkvvfieisdqrlsigspskiydslgqpvfyqasfswdtmikfgd
vltvnplvvnwrnntvisrpgqsqcprfntcpeicwegvyndaflidri
nwisagvfldsnqtaenpvftvfkdneilyraqlasedtnaqktitncf
llknkiwcislveiydtgdnvirpklfavkipeqctggGGGGgvsnlvg
lpnniclqktsnqilkpklisytlpvvgqsgtcitdpllamdegyfays
hlerigscsrgvskqriigvgevldrgdevpslfmtnvwtppnpntvyh
csavynnefyyvlcavstvgdpilnstywsgslmmtrlavkpksngggy
nqhqlalrsiekgrydkvmpygpsgikqgdtlyfpavgflvrtefkynd
sncpitkcqyskpencrlsmgirpnshyilrsgllkynlsdgenpkvvf
ieisdqrlsigspskiydslgqpvfyqasfswdtmikfgdvltvnplvv
nwrnntvisrpgqsqcprfntcpeicwegvyndaflidrinwisagvfl
dsnqtaenpvftvfkdneilyraqlasedtnaqktitncfllknkiwci
slveiydtgdnvirpklfavkipeqctgglvprgshhhhhhsawshpqf
ek

NiVop08-TD (GCN4-Fd)-GG
(SEQ ID NO: 47)
MysmqlascvtltlvllvnsQGILHYEKLSKIGLVKGVTRKYKIKSNPL
TKDIVIKMIPNVSNMSQCTGSVMENYKTRLNGILTPIKGALEIYKNNTH
DCVGDVRLAGVCMAGVAIGIATAAQITAGVALYEAMKNADNINKLKSSI
ESTNEAVVKLQETAEKTVYVFTALQDYINTNLVPTIDKIPCKQTELSLD
LALSKYLSDLLFVFGPNLQDPVSNSMTIQAISQAFGGNYETLLRTLGYA
TEDFDDLLESDSITGQIIYVDLSSYYIIVRVYFPILTEIQQAYIQELLP
VSFNNDNSEWISIVPNFILVRNTLISNIEIGFCLITKRSVICNQDYATP
MTNNMRECLTGSTEKCPRELVVSSHVPRFALSNGVLFANCISVTCQCQT
TGRAISQSGEQTLLMIDNTTCPTAVLGNVIISLGKYLGSVNYNSEGIAI
GPPVFTDKVDISSQISSMNQSLQQSKDYIKEAQRLLDTVNPSLKLMKQI
EDKIEEILSKIYHIENEIARIKKLIGEAPGSGYIPEAPRDGQAYVRKDG
EWVLLSTFLGSGGGGGgvsnlvglpnniclqktsnqilkpklisytlpv
vgqsgtcitdpllamdegyfayshlerigscsrgvskqriigvgevldr
gdevpslfmtnvwtppnpntvyhcsavynnefyyvlcavstvgdpilns
tywsgslmmtrlavkpksngggynqhqlalrsiekgrydkvmpygpsgi
kqgdtlyfpavgflvrtefkyndsncpitkcqyskpencrlsmgirpns
hyilrsgllkynlsdgenpkvvfieisdqrlsigspskiydslgqpvfy
qasfswdtmikfgdvltvnplvvnwrnntvisrpgqsqcprfntcpeic
wegvyndaflidrinwisagvfldsnqtaenpvftvfkdneilyraqla
sedtnaqktitncfllknkiwcislveiydtgdnvirpklfavkipeqc
tggGGGGgvsnlvglpnniclqktsnqilkpklisytlpvvgqsgtcit
dpllamdegyfayshlerigscsrgvskqriigvgevldrgdevpslfm
tnvwtppnpntvyhcsavynnefyyvlcavstvgdpilnstywsgslmm
trlavkpksngggynqhqlalrsiekgrydkvmpygpsgikqgdtlyfp
avgflvrtefkyndsncpitkcqyskpencrlsmgirpnshyilrsgll
kynlsdgenpkvvfieisdqrlsigspskiydslgqpvfyqasfswdtm
ikfgdvltvnplvvnwrnntvisrpgqsqcprfntcpeicwegvyndaf
lidrinwisagvfldsnqtaenpvftvfkdneilyraqlasedtnaqkt
itncfllknkiwcislveiydtgdnvirpklfavkipeqctgglvprgs
hhhhhhsawshpqfek

NiV06-TD (GCN4-Fd)-GGG
(SEQ ID NO: 48)
MysmqlascvtltlvllvnsQGILHYEKLSKIGLVKGVTRKYKIKSNPL
TKDIVIKMIPNVSNMSQCTGSVMENYKTRLNGILTPIKGALEIYKNGGS
GVAIGIATAAQITAGVALYEAMKNADNINKLKSSIESTNEAVVKLQETA
EKTVYVLTALQDYINTNLVPTIDKISCKQTELSLDLALSKYLSDLLFVF
GPNLQDPVSNSMTIQAISQAFGGNYETLLRTLGYATEDFDDLLESDSIT
GQIIYVDLSSYYIIVRVYFPILTEIQQAYIQELLPVSFNNDNSEWISIV
PNFILVRNTLISNIEIGFCLITKRSVICNQDYATPMTNNMRECLTGSTE
KCPRELVVSSHVPRFALSNGVLFANCISVTCQCQTTGRAISQSGEQTLL
MIDNTTCPTAVLGNVIISLGKYLGSVNYNSEGIAIGPPVFTDKVDISSQ
ISSMNQSLQQSKDYIKEAQRLLDTVNPSLKLMKQIEDKIEEILSKIYHI
ENEIARIKKLIGEAPGSGYIPEAPRDGQAYVRKDGEWVLLSTFLGSGGG
GGgvsnlvglpnniclqktsnqilkpklisytlpvvgqsgtcitdplla
mdegyfayshlerigscsrgvskqriigvgevldrgdevpslfmtnvwt
ppnpntvyhcsavynnefyyvlcavstvgdpilnstywsgslmmtrlav
kpksngggynqhqlalrsiekgrydkvmpygpsgikqgdtlyfpavgfl
vrtefkyndsncpitkcqyskpencrlsmgirpnshyilrsgllkynls
dgenpkvvfieisdqrlsigspskiydslgqpvfyqasfswdtmikfgd
vltvnplvvnwrnntvisrpgqsqcprfntcpeicwegvyndaflidri
nwisagvfldsnqtaenpvftvfkdneilyraqlasedtnaqktitncf
llknkiwcislveiydtgdnvirpklfavkipeqctggGGGGgvsnlvg
lpnniclqktsnqilkpklisytlpvvgqsgtcitdpllamdegyfays
hlerigscsrgvskqriigvgevldrgdevpslfmtnvwtppnpntvyh
csavynnefyyvlcavstvgdpilnstywsgslmmtrlavkpksngggy
nqhqlalrsiekgrydkvmpygpsgikqgdtlyfpavgflvrtefkynd
sncpitkcqyskpencrlsmgirpnshyilrsgllkynlsdgenpkvvf
ieisdqrlsigspskiydslgqpvfyqasfswdtmikfgdvltvnplvv
nwrnntvisrpgqsqcprfntcpeicwegvyndaflidrinwisagvfl
dsnqtaenpvftvfkdneilyraqlasedtnaqktitncfllknkiwci
slveiydtgdnvirpklfavkipeqctggGGGGgvsnlvglpnniclqk
tsnqilkpklisytlpvvgqsgtcitdpllamdegyfayshlerigscs
rgvskqriigvgevldrgdevpslfmtnvwtppnpntvyhcsavynnef
yyvlcavstvgdpilnstywsgslmmtrlavkpksngggynqhqlalrs
iekgrydkvmpygpsgikqgdtlyfpavgflvrtefkyndsncpitkcq
yskpencrlsmgirpnshyilrsgllkynlsdgenpkvvfieisdqrls
igspskiydslgqpvfyqasfswdtmikfgdvltvnplvvnwrnntvis
rpgqsqcprfntcpeicwegvyndaflidrinwisagvfldsnqtaenp
vftvfkdneilyraqlasedtnaqktitncfllknkiwcislveiydtg
dnvirpklfavkipeqctgglvprgshhhhhhsawshpqfek

NiVop08-TD(GCN4-Fd)-GGG
(SEQ ID NO: 49)
MysmqlascvtltlvllvnsQGILHYEKLSKIGLVKGVTRKYKIKSNPL
TKDIVIKMIPNVSNMSQCTGSVMENYKTRLNGILTPIKGALEIYKNNTH
DCVGDVRLAGVCMAGVAIGIATAAQITAGVALYEAMKNADNINKLKSSI
ESTNEAVVKLQETAEKTVYVFTALQDYINTNLVPTIDKIPCKQTELSLD
LALSKYLSDLLFVFGPNLQDPVSNSMTIQAISQAFGGNYETLLRTLGYA
TEDFDDLLESDSITGQIIYVDLSSYYIIVRVYFPILTEIQQAYIQELLP
VSFNNDNSEWISIVPNFILVRNTLISNIEIGFCLITKRSVICNQDYATP
MTNNMRECLTGSTEKCPRELVVSSHVPRFALSNGVLFANCISVTCQCQT
TGRAISQSGEQTLLMIDNTTCPTAVLGNVIISLGKYLGSVNYNSEGIAI
GPPVFTDKVDISSQISSMNQSLQQSKDYIKEAQRLLDTVNPSLKLMKQI
EDKIEEILSKIYHIENEIARIKKLIGEAPGSGYIPEAPRDGQAYVRKDG
EWVLLSTFLGSGGGGGgvsnlvglpnniclqktsnqilkpklisytlpv
vgqsgtcitdpllamdegyfayshlerigscsrgvskqriigvgevldr
gdevpslfmtnvwtppnpntvyhcsavynnefyyvlcavstvgdpilns
tywsgslmmtrlavkpksngggynqhqlalrsiekgrydkvmpygpsgi
kqgdtlyfpavgflvrtefkyndsncpitkcqyskpencrlsmgirpns
hyilrsgllkynlsdgenpkvvfieisdqrlsigspskiydslgqpvfy
qasfswdtmikfgdvltvnplvvnwrnntvisrpgqsqcprfntcpeic
wegvyndaflidrinwisagvfldsnqtaenpvftvfkdneilyraqla
sedtnaqktitncfllknkiwcislveiydtgdnvirpklfavkipeqc
tggGGGGgvsnlvglpnniclqktsnqilkpklisytlpvvgqsgtcit
dpllamdegyfayshlerigscsrgvskqriigvgevldrgdevpslfm
tnvwtppnpntvyhcsavynnefyyvlcavstvgdpilnstywsgslmm
trlavkpksngggynqhqlalrsiekgrydkvmpygpsgikqgdtlyfp
avgflvrtefkyndsncpitkcqyskpencrlsmgirpnshyilrsgll
kynlsdgenpkvvfieisdqrlsigspskiydslgqpvfyqasfswdtm
ikfgdvltvnplvvnwrnntvisrpgqsqcprfntcpeicwegvyndaf
lidrinwisagvfldsnqtaenpvftvfkdneilyraqlasedtnaqkt
itncfllknkiwcislveiydtgdnvirpklfavkipeqctggGGGGgv
snlvglpnniclqktsnqilkpklisytlpvvgqsgtcitdpllamdeg
yfayshlerigscsrgvskqriigvgevldrgdevpslfmtnvwtppnp
ntvyhcsavynnefyyvlcavstvgdpilnstywsgslmmtrlavkpks
ngggynqhqlalrsiekgrydkvmpygpsgikqgdtlyfpavgflvrte
fkyndsncpitkcqyskpencrlsmgirpnshyilrsgllkynlsdgen
pkvvfieisdqrlsigspskiydslgqpvfyqasfswdtmikfgdvltv
nplvvnwrnntvisrpgqsqcprfntcpeicwegvyndaflidrinwis
agvfldsnqtaenpvftvfkdneilyraqlasedtnaqktitncfllkn
kiwcislveiydtgdnvirpklfavkipeqctgglvprgshhhhhhsaw
shpqfek

G-NiV06-TD(GCN4-Fd)
(SEQ ID NO: 50)
MysmqlascvtltlvllvnsQrpqtegvsnlvglpnniclqktsnqilk
pklisytlpvvgqsgtcitdpllamdegyfayshlerigscsrgvskqr
iigvgevldrgdevpslfmtnvwtppnpntvyhcsavynnefyyvlcav
stvgdpilnstywsgslmmtrlavkpksngggynqhqlalrsiekgryd
kvmpygpsgikqgdtlyfpavgflvrtefkyndsncpitkcqyskpenc
rlsmgirpnshyilrsgllkynlsdgenpkvvfieisdqrlsigspski
ydslgqpvfyqasfswdtmikfgdvltvnplvvnwrnntvisrpgqsqc
prfntcpeicwegvyndaflidrinwisagvfldsnqtaenpvftvfkd
neilyraqlasedtnaqktitncfllknkiwcislveiydtgdnvirpk
lfavkipeqctgggGGQGILHYEKLSKIGLVKGVTRKYKIKSNPLTKDI
VIKMIPNVSNMSQCTGSVMENYKTRLNGILTPIKGALEIYKNGGSGVAI
GIATAAQITAGVALYEAMKNADNINKLKSSIESTNEAVVKLQETAEKTV
YVLTALQDYINTNLVPTIDKISCKQTELSLDLALSKYLSDLLFVFGPNL
QDPVSNSMTIQAISQAFGGNYETLLRTLGYATEDFDDLLESDSITGQII
YVDLSSYYIIVRVYFPILTEIQQAYIQELLPVSFNNDNSEWISIVPNFI
LVRNTLISNIEIGFCLITKRSVICNQDYATPMTNNMRECLTGSTEKCPR
ELVVSSHVPRFALSNGVLFANCISVTCQCQTTGRAISQSGEQTLLMIDN
TTCPTAVLGNVIISLGKYLGSVNYNSEGIAIGPPVFTDKVDISSQISSM
NQSLQQSKDYIKEAQRLLDTVNPSLklmkqiedkieeilskiyhienei
arikkligeapgglvprgshhhhhhsawshpqfek

G-NiVop08-TD(GCN4-Fd)
(SEQ ID NO: 51)
MysmqlascvtltlvllvnsQrpqtegvsnlvglpnniclqktsnqilk
pklisytlpvvgqsgtcitdpllamdegyfayshlerigscsrgvskqr
iigvgevldrgdevpslfmtnvwtppnpntvyhcsavynnefyyvlcav
stvgdpilnstywsgslmmtrlavkpksngggynqhqlalrsiekgryd
kvmpygpsgikqgdtlyfpavgflvrtefkyndsncpitkcqyskpenc
rlsmgirpnshyilrsgllkynlsdgenpkvvfieisdqrlsigspski
ydslgqpvfyqasfswdtmikfgdvltvnplvvnwrnntvisrpgqsqc
prfntcpeicwegvyndaflidrinwisagvfldsnqtaenpvftvfkd
neilyraqlasedtnaqktitncfllknkiwcislveiydtgdnvirpk
lfavkipeqctggggsggggQGILHYEKLSKIGLVKGVTRKYKIKSNPL
TKDIVIKMIPNVSNMSQCTGSVMENYKTRLNGILTPIKGALEIYKNNTH
DCVGDVRLAGVCMAGVAIGIATAAQITAGVALYEAMKNADNINKLKSSI
ESTNEAVVKLQETAEKTVYVFTALQDYINTNLVPTIDKIPCKQTELSLD
LALSKYLSDLLFVFGPNLQDPVSNSMTIQAISQAFGGNYETLLRTLGYA
TEDFDDLLESDSITGQIIYVDLSSYYIIVRVYFPPRELVVSSHVPRFAL
SNGVLFANCISVTCQCQTTGRAISQSGEQTLLMIDNTTCPTAVLGNVII
SLGKYLGSVNYNSEGIAIGPPVFTDKVDISSQISSMNQSLQQSKDYIKE
AQRLLDTVNPSLKLMKQIEDKIEEILSKIYHIENEIARIKKLIGEAPGS
GYIPEAPRDGQAYVRKDGEWVLLSTFLGSLVPRGSHHHHHHSAWSHPQF
EK

NiVop08-GCN4-G
(SEQ ID NO: 59)
MYSMQLASCVTLTLVLLVNSQGILHYEKLSKIGLVKGVTRKYKIKSNPL
TKDIVIKMIPNVSNMSQCTGSVMENYKTRLNGILTPIKGALEIYKNNTH
DCVGDVRLAGVCMAGVAIGIATAAQITAGVALYEAMKNADNINKLKSSI
ESTNEAVVKLQETAEKTVYVFTALQDYINTNLVPTIDKIPCKQTELSLD
LALSKYLSDLLFVFGPNLQDPVSNSMTIQAISQAFGGNYETLLRTLGYA
TEDFDDLLESDSITGQIIYVDLSSYYIIVRVYFPILTEIQQAYIQELLP
VSFNNDNSEWISIVPNFILVRNTLISNIEIGFCLITKRSVICNQDYATP
MTNNMRECLTGSTEKCPRELVVSSHVPRFALSNGVLFANCISVTCQCQT
TGRAISQSGEQTLLMIDNTTCPTAVLGNVIISLGKYLGSVNYNSEGIAI
GPPVFTDKVDISSQISSMNQSLQQSKDYIKEAQRLLDTVNPSLKLMKQI
EDKIEEILSKIYHIENEIARIKKLIGEAPGSGGGGGGVSNLVGLPNNIC
LQKTSNQILKPKLISYTLPVVGQSGTCITDPLLAMDEGYFAYSHLERIG
SCSRGVSKQRIIGVGEVLDRGDEVPSLFMTNVWTPPNPNTVYHCSAVYN
NEFYYVLCAVSTVGDPILNSTYWSGSLMMTRLAVKPKSNGGGYNQHQLA
LRSIEKGRYDKVMPYGPSGIKQGDTLYFPAVGFLVRTEFKYNDSNCPIT
KCQYSKPENCRLSMGIRPNSHYILRSGLLKYNLSDGENPKVVFIEISDQ
RLSIGSPSKIYDSLGQPVFYQASFSWDTMIKFGDVLTVNPLVVNWRNNT
VISRPGQSQCPRFNTCPEICWEGVYNDAFLIDRINWISAGVFLDSNQTA
ENPVFTVFKDNEILYRAQLASEDTNAQKTITNCFLLKNKIWCISLVEIY
DTGDNVIRPKLFAVKIPEQCTGGLVPRGSHHHHHHSAWSHPQFEK

NiVop08-Fd-G
(SEQ ID NO: 60)
MYSMQLASCVTLTLVLLVNSQGILHYEKLSKIGLVKGVTRKYKIKSNPL
TKDIVIKMIPNVSNMSQCTGSVMENYKTRLNGILTPIKGALEIYKNNTH
DCVGDVRLAGVCMAGVAIGIATAAQITAGVALYEAMKNADNINKLKSSI
ESTNEAVVKLQETAEKTVYVFTALQDYINTNLVPTIDKIPCKQTELSLD
LALSKYLSDLLFVFGPNLQDPVSNSMTIQAISQAFGGNYETLLRTLGYA
TEDFDDLLESDSITGQIIYVDLSSYYIIVRVYFPILTEIQQAYIQELLP
VSFNNDNSEWISIVPNFILVRNTLISNIEIGFCLITKRSVICNQDYATP
MTNNMRECLTGSTEKCPRELVVSSHVPRFALSNGVLFANCISVTCQCQT
TGRAISQSGEQTLLMIDNTTCPTAVLGNVIISLGKYLGSVNYNSEGIAI
GPPVFTDKVDISSQISSMNQSLQQSKDYIKEAQRLLDTVNPSLGSGYIP
EAPRDGQAYVRKDGEWVLLSTFLGSGGGGGGVSNLVGLPNNICLQKTSN
QILKPKLISYTLPVVGQSGTCITDPLLAMDEGYFAYSHLERIGSCSRGV
SKQRIIGVGEVLDRGDEVPSLFMTNVWTPPNPNTVYHCSAVYNNEFYYV
LCAVSTVGDPILNSTYWSGSLMMTRLAVKPKSNGGGYNQHQLALRSIEK
GRYDKVMPYGPSGIKQGDTLYFPAVGFLVRTEFKYNDSNCPITKCQYSK
PENCRLSMGIRPNSHYILRSGLLKYNLSDGENPKVVFIEISDQRLSIGS
PSKIYDSLGQPVFYQASFSWDTMIKFGDVLTVNPLVVNWRNNTVISRPG
QSQCPRFNTCPEICWEGVYNDAFLIDRINWISAGVFLDSNQTAENPVFT
VFKDNEILYRAQLASEDTNAQKTITNCFLLKNKIWCISLVEIYDTGDNV
IRPKLFAVKIPEQCTGGLVPRGSHHHHHHSAWSHPQFEK

The above sequences include F and G ectodomains, an N-terminal signal peptide, a HIS tag, a Strep tag, and a thrombin cleavage site to remove the two tags, and various linker residues between segments.

The NiVop08-TD-G (SEQ ID NO: 43), G-NiVop08-T (SEQ ID NO: 44), and NiV06-TD-G (SEQ ID NO: 45) constructs were expressed in cells and purified as discussed above for soluble NiV F ectodomain trimers in Example 1. Additional constructs based on NiVop08 that included a trimerization domain with GCN4 or Fd (but not both) were also expressed in cell and purified as discussed above for soluble NiV F ectodomain trimers in Example 1. Each of the constructs was successfully purified as a chimeric multimer. Further, negative stain EM showed that all of these constructs formed multimers. Exemplary negative stain EM images are shown in FIGS. 7B and 7C. These EM assessment shows that the pre-F-constructs contain viral fusion proteins in the prefusion form and that the molecules have three additional round G domains at the end of the tail for F-TD-G constructs and near the head for G-F-TD constructs (arrows show examples). There are some variations in the G positions since the tails are flexible.

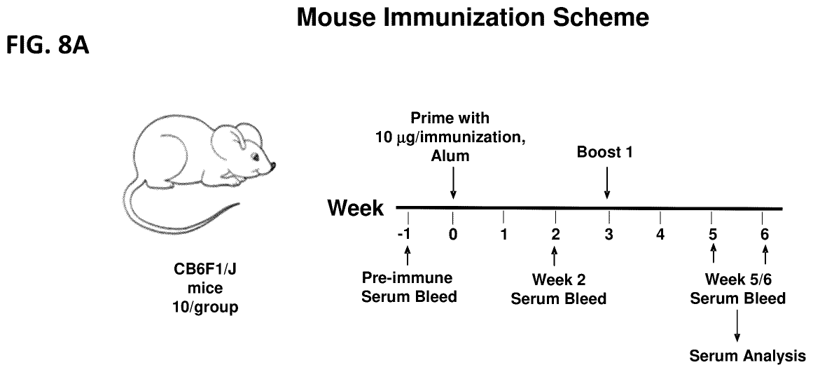

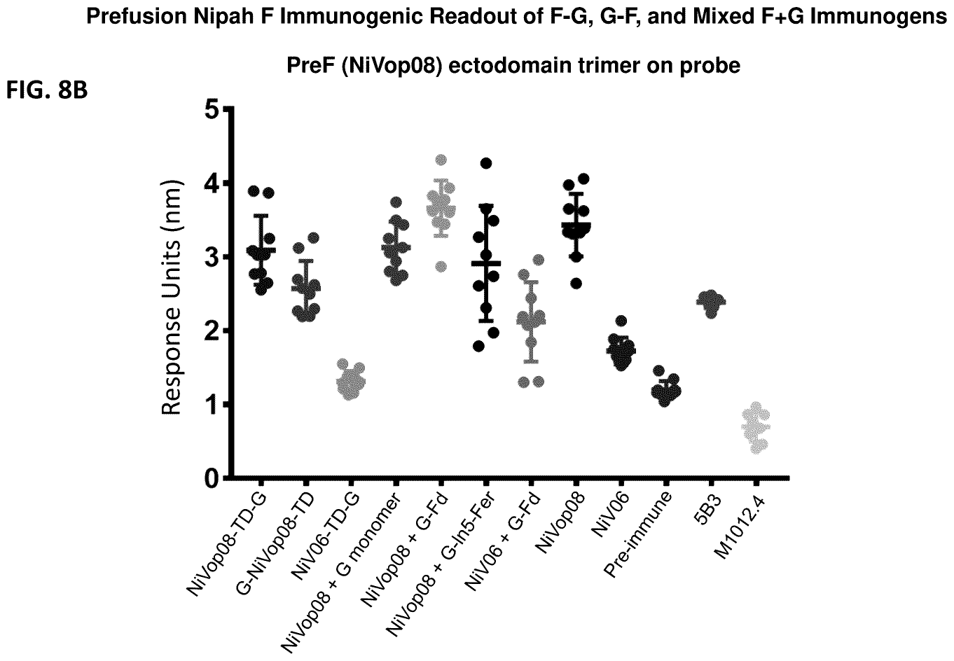

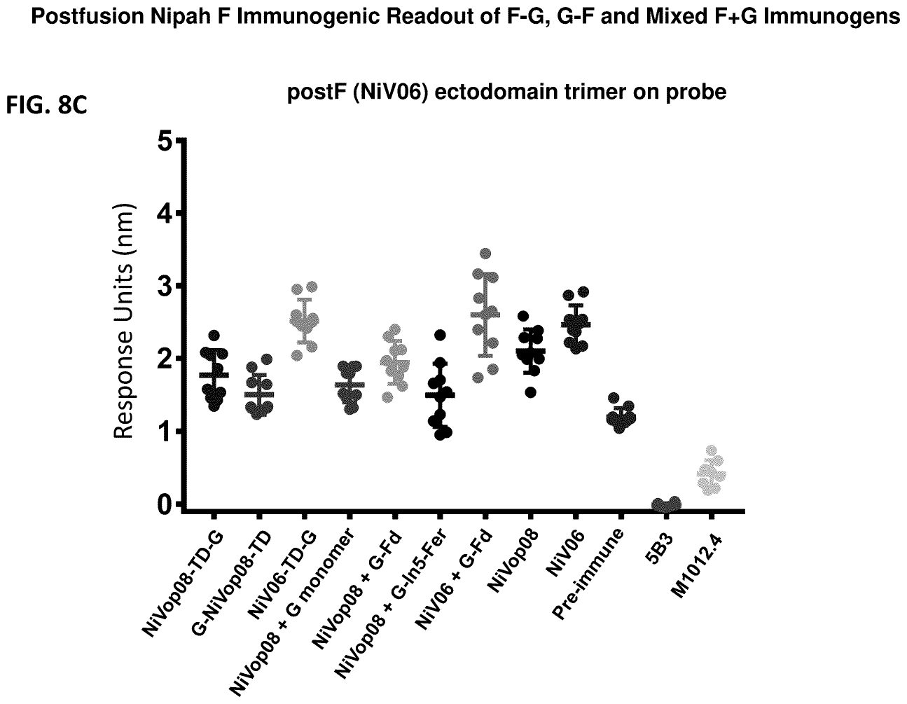

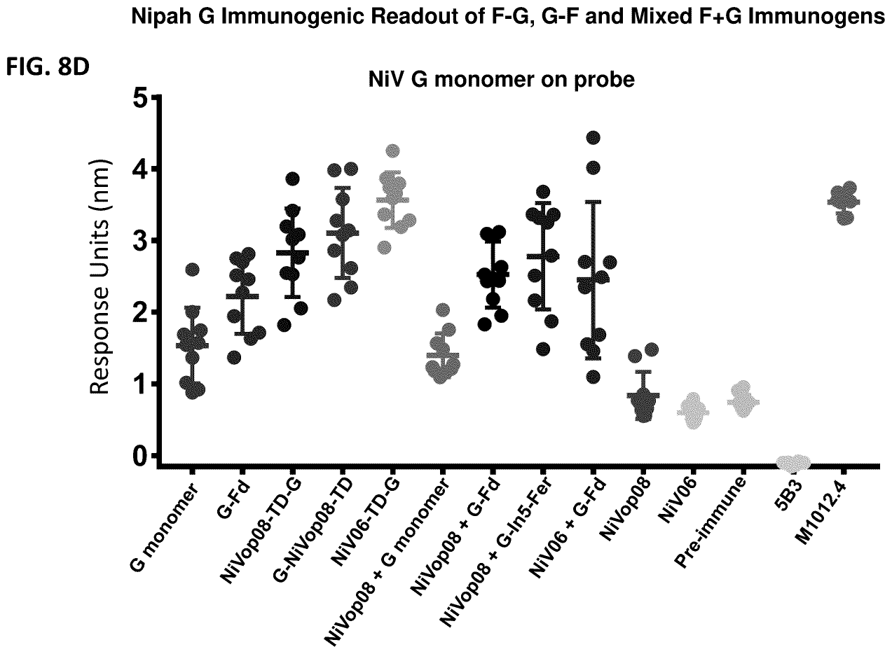

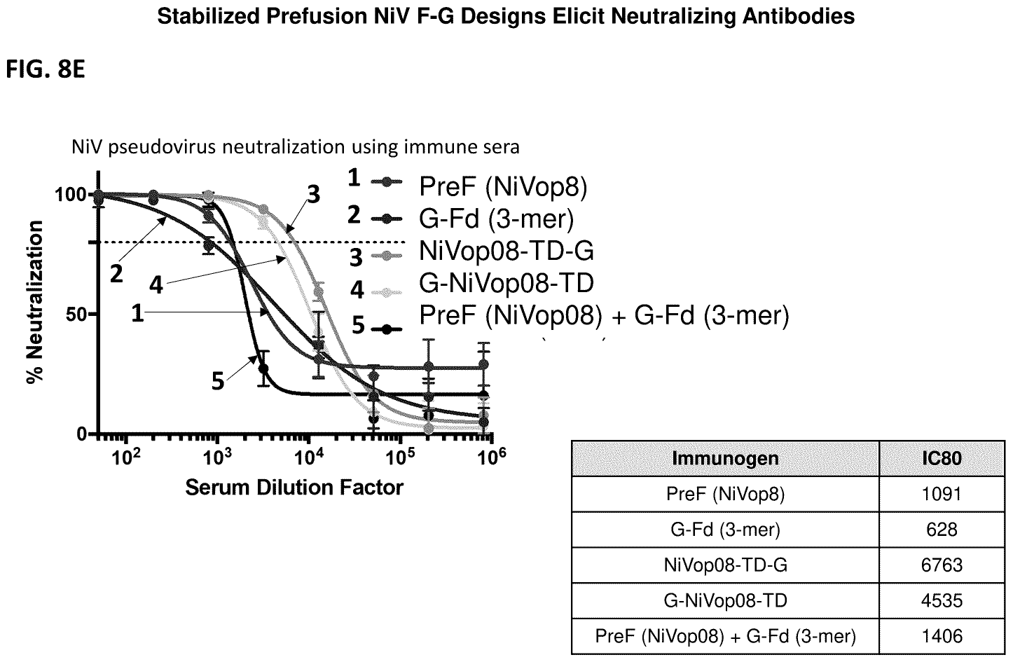

Immunization assays were conducted with the F-G chimeric multimers, and other constructs described herein. The immunization assays were performed substantially as described in Example 1. CB6F1/J mice were immunized with 10 μg of total protein in Alum (10 g for single immunogen assays, or 5 μg each for immunizations including two immunogens) using the schedule shown in FIG. 8A. Sera from immunized mice was tested for binding to prefusion F ectodomain trimer (NiVop08), postfusion F ectodomain trimer (NiV06), or monomeric NiV G using an Octet binding assay (see FIGS. 8A-8C). The immune sera was also assessed in a NiV pseudovirus neutralization assay (FIG. 8E), which showed that immune sera from animals treated with the multimeric NiV F-G constructs neutralized NiV.

### Example 4

### RNA and Protein Immunization in a Ferret Model

This example describes results from the immunization of ferrets with several of the disclosed NiV immunogens.

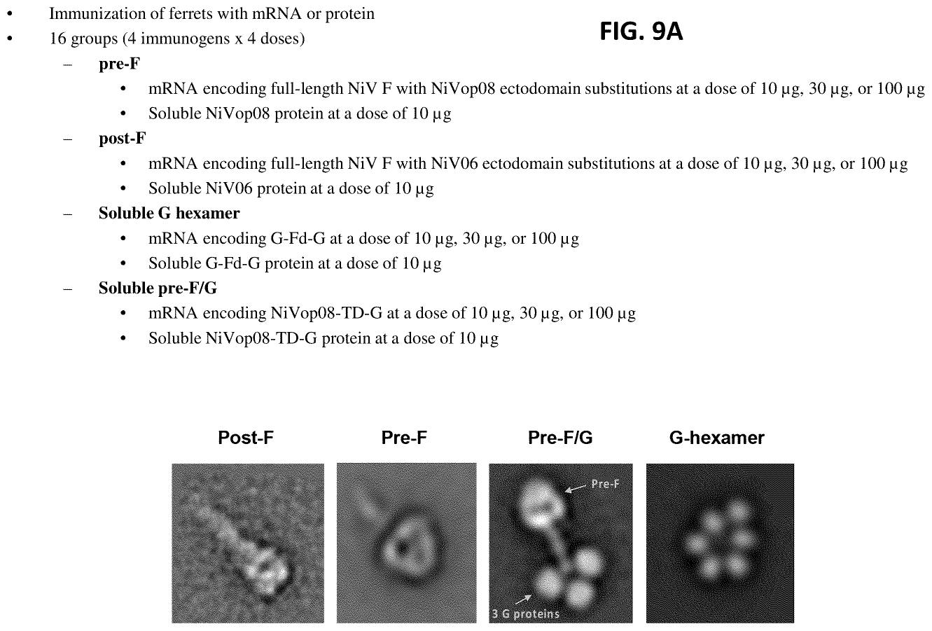

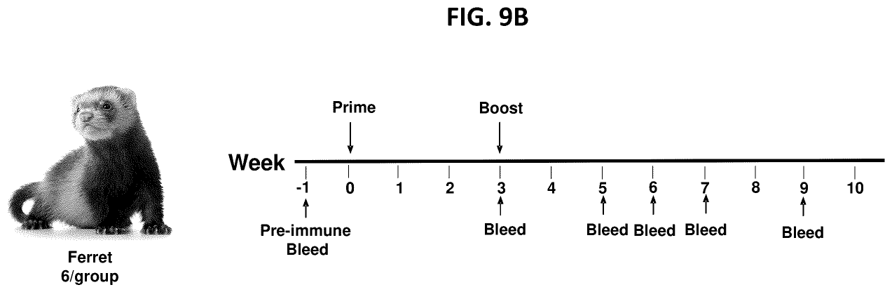

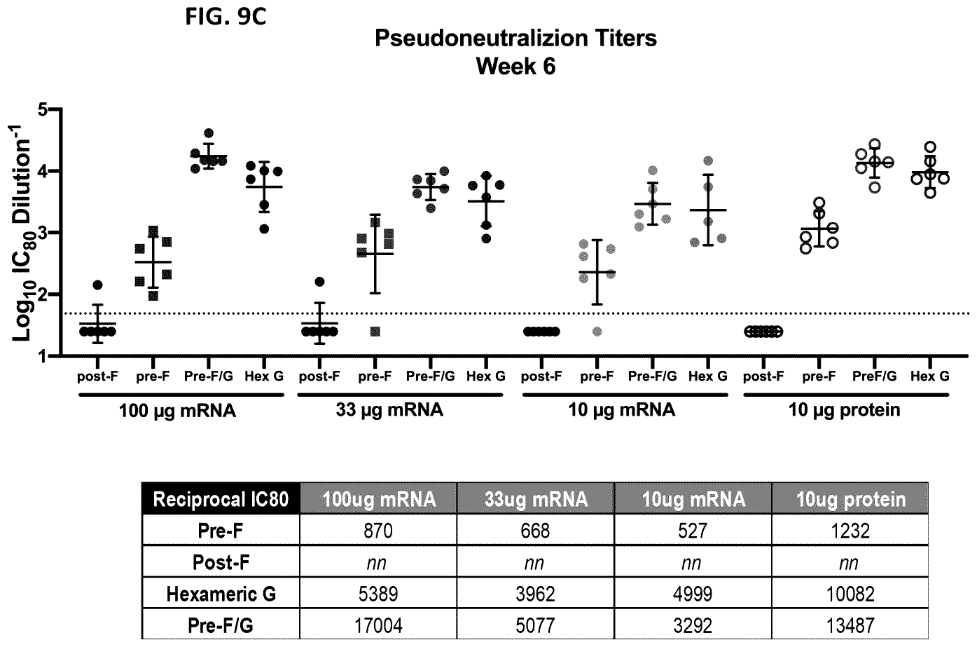

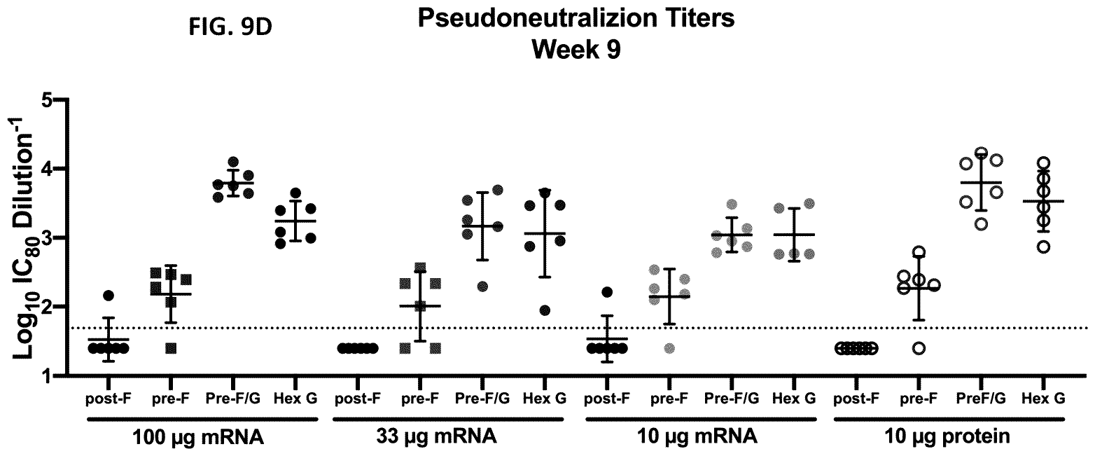

Ferrets were immunized with the preF, postF, G hexamer, or preF/G chimera immunogen based on a protein or RNA platform in 16 different groups according to the schedule shown in FIGS. 9A and 9B. The immunogens used were:
(1) preF: immunization with 10 μg mRNA encoding full-length NiV F with NiVop08 ectodomain substitutions (L104C-I114C, L172F, S191P).
(2) preF: immunization with 30 μg mRNA encoding full-length NiV F with NiVop08 ectodomain substitutions (L104C-I114C, L172F, S191P).
(3) preF: immunization with 100 μg mRNA encoding full-length NiV F with NiVop08 ectodomain substitutions (L104C-I114C, L172F, S191P).
(4) preF: immunization with 10 μg purified soluble NiVop08 protein.
(5) postF: immunization with 10 μg mRNA encoding full-length NiV F with NiV06 ectodomain substitutions (A100-116, residues N99-G117 linked by a GGS linker).
(6) postF: immunization with 30 μg mRNA encoding full-length NiV F with NiV06 ectodomain substitutions (A100-116, residues N99-G117 linked by a GGS linker).
(7) postF: immunization with 100 μg mRNA encoding full-length NiV F with NiV06 ectodomain substitutions (A100-116, residues N99-G117 linked by a GGS linker).
(8) postF: immunization with 10 μg purified soluble NiV06 protein.
(9) soluble G hexamer: immunization with 10 μg mRNA encoding G-Fd-G (SEQ ID NO: 37).
(10) soluble G hexamer: immunization with 30 μg mRNA encoding G-Fd-G (SEQ ID NO: 37).
(11) soluble G hexamer: immunization with 100 μg mRNA encoding G-Fd-G (SEQ ID NO: 37).
(12) soluble G hexamer: immunization with 10 μg purified soluble trimeric G-Fd-G (SEQ ID NO: 37) protein.
(13) soluble preF/G chimera: immunization with 10 μg mRNA encoding NiVop08-TD(GCN4-Fd)-G (SEQ ID NO: 43).
(14) soluble preF/G chimera: immunization with 30 μg mRNA encoding NiVop08-TD(GCN4-Fd)-G (SEQ ID NO: 43).
(15) soluble preF/G chimera: immunization with 100 μg mRNA encoding NiVop08-TD(GCN4-Fd)-G (SEQ ID NO: 43).
(16) soluble preF/G chimera: immunization with 10 μg purified soluble trimeric NiVop08-TD(GCN4-Fd)-G (SEQ ID NO: 43) protein.

Protein immunizations were performed as described above. RNA immunizations were performed with mRNA encoding the new immunogens using a lipid-encapsulated mRNA immunization platform substantially as previously described (see Roth et al., “A Modified mRNA Vaccine Targeting Immunodominant NS Epitopes Protects Against Dengue Virus Infection in HLA Class I Transgenic Mice,” Frot Immunol., Jun. 21, 2019, Vol. 10, Article 1424; and Jagger et al., J Infect Dis, “Protective Efficacy of Nucleic Acid Vaccines Against Transmission of Zika Virus During Pregnancy in Mice,” jiz338, Jul. 1, 2019).

Sera was collected from the immunized animals at multiple time points. Sera from three and six weeks following the second dose was assessed for NiV neutralization using the pseudovirus neutralization assay described above (FIGS. 9C and 9D).

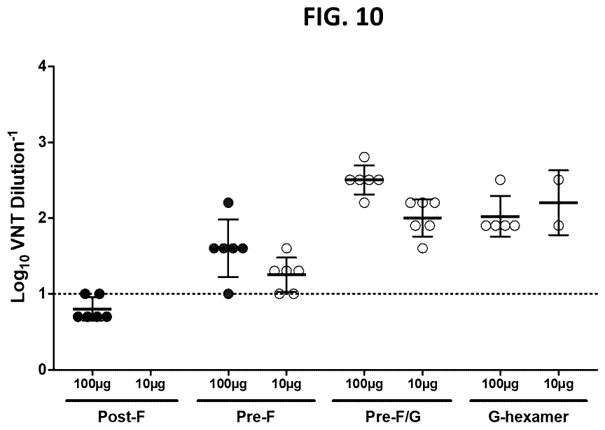

An in vitro virus neutralization test (VNT) using live NiV infection of cells was performed with sera from the 10 μg and 100 μg mRNA immunization conditions using the preF, preF/G chimera, and G-hexamer immunogens (FIG. 10). As shown, sera from each of the immunization conditions neutralized the live NiV infection of cells in vitro.

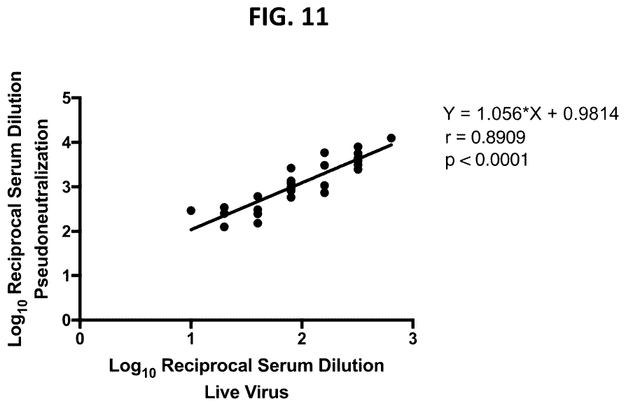

The results of the pseudovirus neutralization assays were compared to the results of the VNT assays to determine if the immune sera neutralized live- and pseudo-NiV to a similar extent. The neutralization of NiV pseudovirus by sera from the preF, preF/G chimera, and G-hexamer immunization conditions correlated well with the neutralization of live NiV infection of cells in the VNT assay by the same sera. FIG. 11 shows an exemplary linear regression graph depicting the correlation of live and pseudovirus NiV neutralization.

### Example 5

### Immunogen Thermal Stability

This example provides the results of assays to ascertain the thermal stability of the preF, postF, preF/G chimera, and G-hexamer immunogens.

Three separate assays were used to interrogate the thermal stability of these immunogens: Differential Scanning calorimetry (DSC), Intrinsic Fluorescence spectral analysis, and Dynamic Light Scattering (DLS). DSC detects all thermally induced transitions, while other technique help interpret those transitions as conformational or colloidal changes in the molecule. For all proteins except PostF, the initial thermal event was linked to tertiary structure changes and/or aggregation. The following table provides the transition midpoint (Tₘ in ° C.) for different thermal transitions identified using the DSC, Intrinsic Fluorescence, and DLS assays for the PreF (NiVop08), PostF (NiV06), HexG (G-Fd-G, SEQ ID NO: 37), and PreF-G (NiVop08-TD-G, SEQ ID NO: 43) immunogens. The results show that each of these immunogens is quite stable at temperatures below 35° C., which is comparable to other subunit vaccines that are suitable for clinical use.

| Technique | Reportable | Sample | Event A | Event B | Event C | Event D |
|---|---|---|---|---|---|---|
| Differential | Transition | PreF | 54.6 | 60.6 | ND | ND |
| Scanning | Midpoint | PostF | ND | ND | ND | 94.0 |
| Calorimetry | (Tₘ) | HexG | ND | 58.7 | 65.4 | ND |
| | | PreF-G | 52.0 | 60.8 | 65.7 | ND |
| Intrinsic | Transition | PreF | ND | 60.3 | ND | ND |
| Fluor. | Midpoint | PostF | ND | ND | ND | ND |
| | (Tₘ ) | HexG | ND | 62.8 | ND | ND |
| | | PreF-G | ND | ND | 71.5 | ND |
| Dynamic | Transition | PreF | ND | 56.8 | ND | ND |
| Light | Onset | PostF | ND | ND | ND | ND |
| Scattering | (Tₒₙₛₑₜ) | HexG | ND | 60.5 | ND | ND |
| (DLS) | | PreF-G | ND | 57.8 | ND | ND |

ND: No transition detected using standard analytical parameters.

### Example 6

### Immunogen Comparison: Signal Sequence and Transmembrane Vs Soluble mRNA

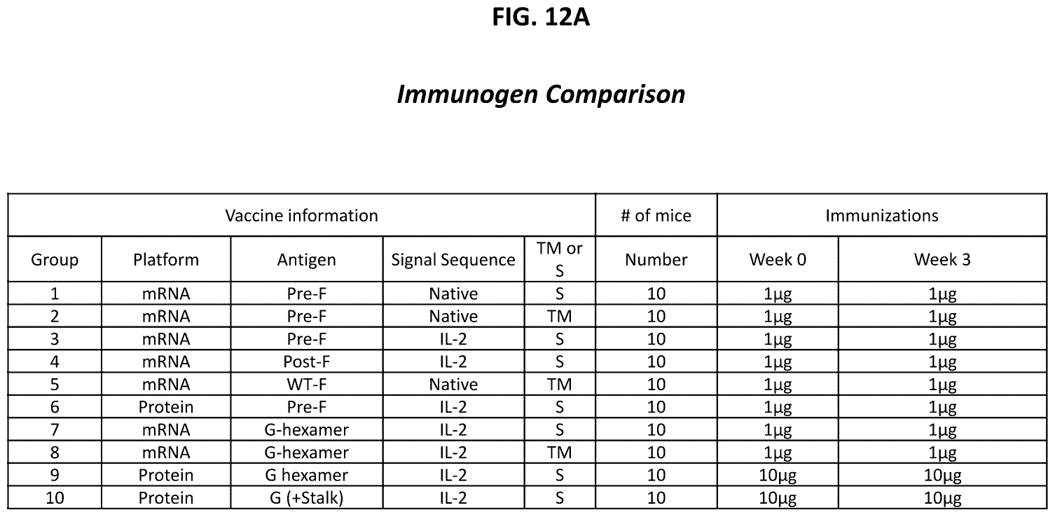

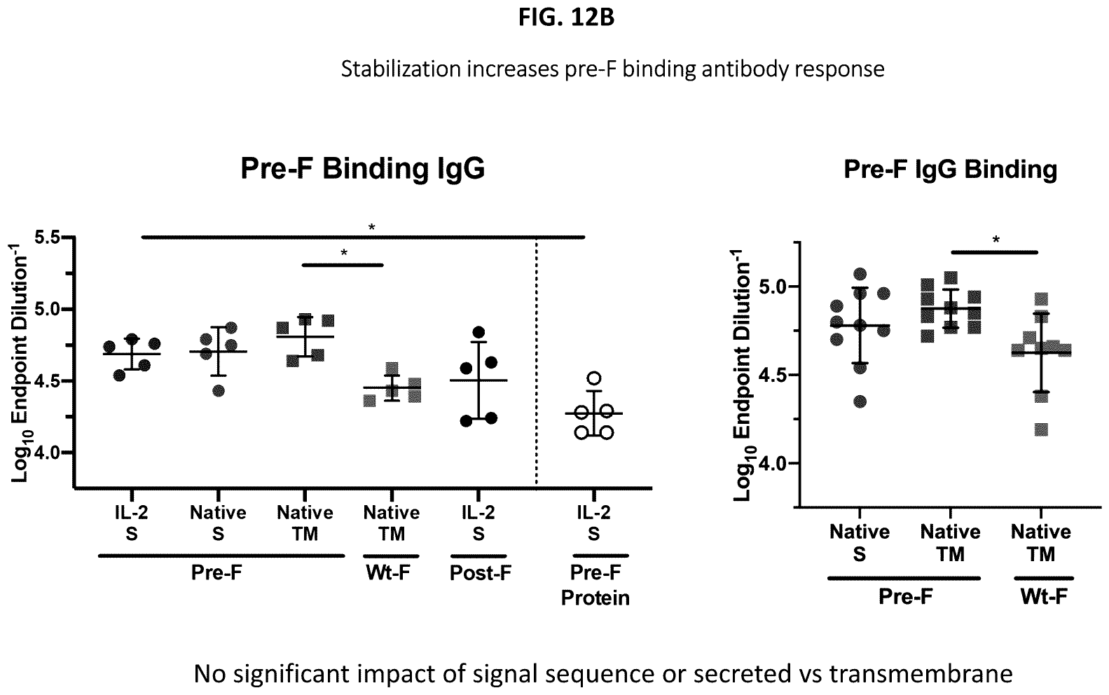

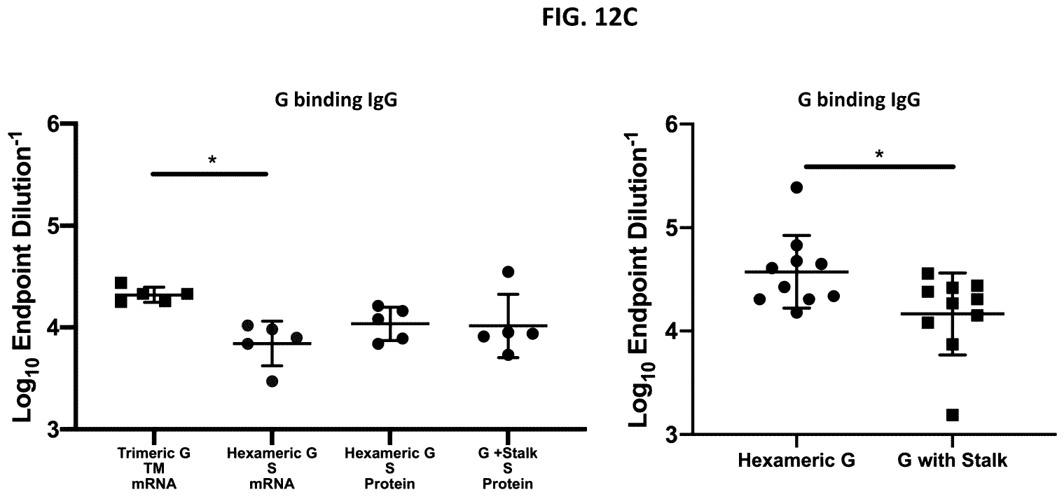

This example describes a comparison of different immunogen variations for eliciting an immune response to Nipah virus in an animal model. Variables assessed include prefusion stabilized vs. wild-type (WT) NiV F, NiV G as a trimer, hexamer, and tetramer (with stalk), transmembrane (TM) vs soluble/secreted NiV F and G, signal sequence (IL-2 signal sequence or native NiV signal sequence), and mRNA vs. protein immunization (See FIG. 12A). The native G ectodomain including both the stalk and head regions forms a tetramer (similar to the Hendra G vaccine approved for veterinary use).

Protein and mRNA immunizations were performed as described above and sera collected from the immunized animals was assessed for NiV preF and G binding. The results (FIGS. 12B and 12C) show that immunization with a prefusion stabilized NiV F increases elicitation of pre-F binding antibody, that there was no significant impact of signal sequence or secreted vs transmembrane conditions, that trimeric G membrane-anchored is a little more immunogenic than hexameric G secreted, and that the G-hexamer immunogen is somewhat more immunogenic than the tetrameric G including stalk.

### Example 7

### RNA and Protein Immunization in a Mouse Model

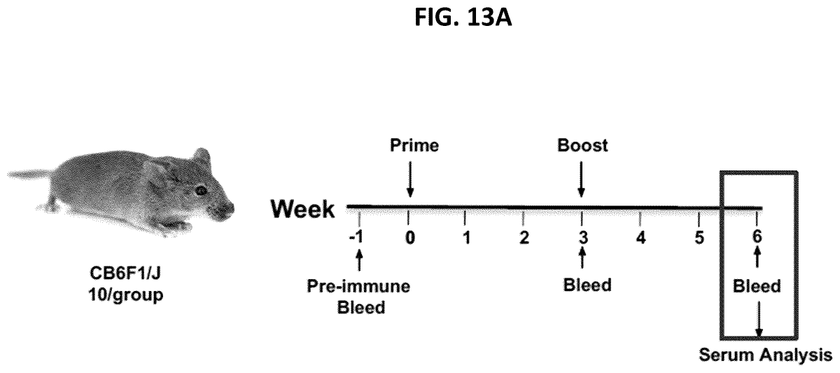

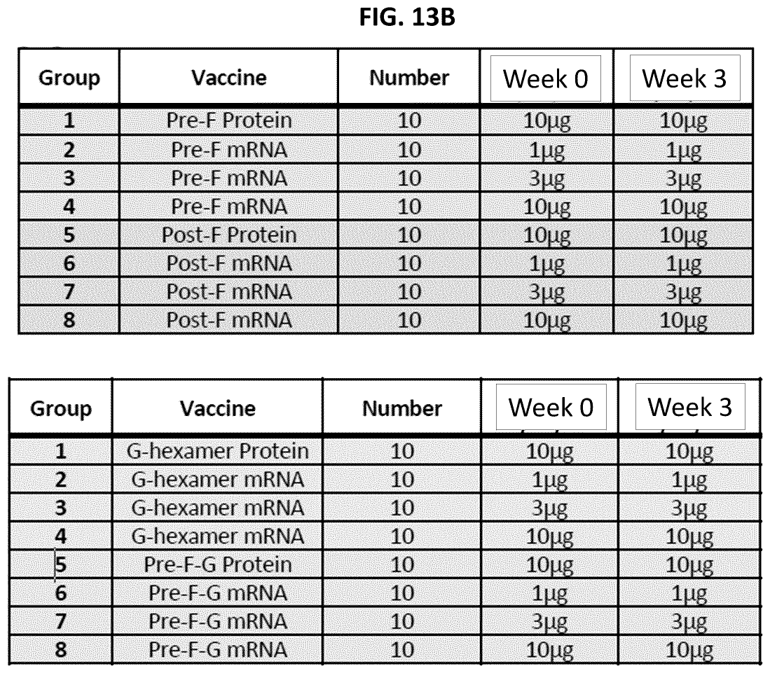

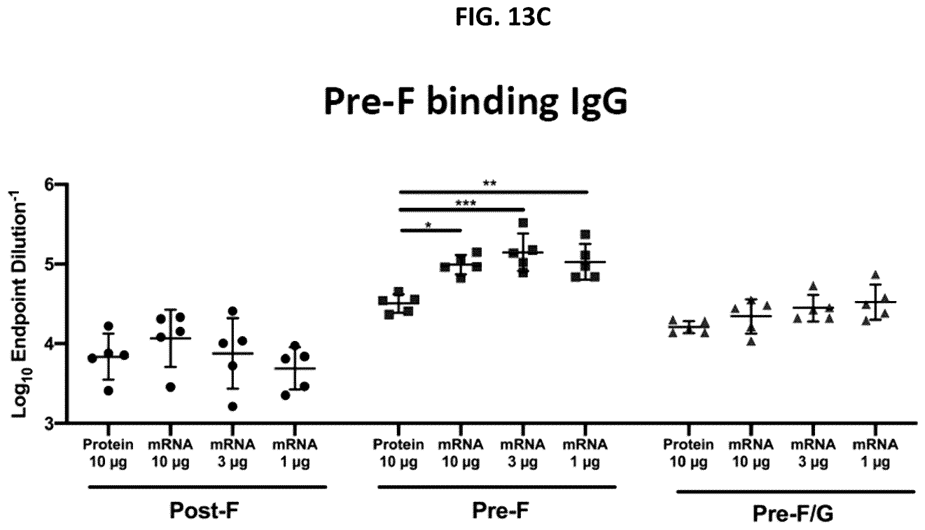

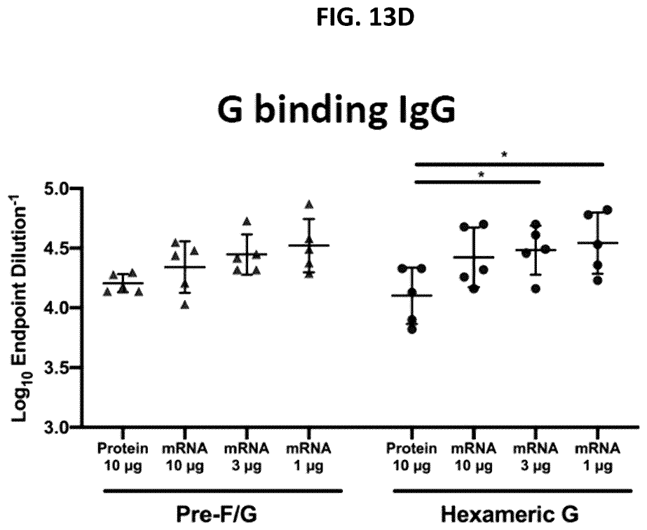

This example describes a comparison of different immunogens and dosages for eliciting an immune response to Nipah virus in a mouse model. The preF (NiVop08), postF (NiV06), G-hexamer (also referred to as hexG; G-Fd-G, SEQ ID NO: 37), and PreF-G (NiVop08-TD-G, SEQ ID NO: 43) immunogens were assessed using mRNA and protein-based immunization protocols as described above and sera collected from the immunized animals was assessed for NiV preF and G binding. The immunization scheme and summary is provided in FIGS. 13A and 13B. The results (FIGS. 13C and 13D) show that both mRNA and protein based immunizations elicited an immune response in mice.

It will be apparent that the precise details of the methods or compositions described may be varied or modified without departing from the spirit of the described embodiments. We claim all such modifications and variations that fall within the scope and spirit of the claims below.

It is claimed:

1. An immunogen, comprising:
a trimer of fusion proteins, each fusion protein comprising, in an N- to C-terminal direction:
one or more NiV G ectodomains and a trimerization domain;
a trimerization domain and one or more NiV G ectodomains; or
one or more NiV G ectodomains, a trimerization domain, and one or more NiV G ectodomains; and wherein the trimerization domains interact to form the trimer; or
a protein nanoparticle comprising one or more NiV G ectodomains extending radially outward from a globular surface of the nanoparticle, the protein nanoparticle comprising self-assembling protein nanoparticle subunits, wherein each subunit is fused N-terminally to the one or more NiV G ectodomain.

2. The immunogen of claim 1, wherein the trimerization domain comprises a GCN4 trimerization domain, a T4 fibritin trimerization domain, or a GCN4 trimerization domain and a T4 fibritin trimerization domain.

3. The immunogen of claim 2, wherein the GCN4 trimerization domain comprises an amino acid sequence set forth as KLMKQIEDKIEEILSKIYHIENEIARIKKLIGEAP (residues 485-519 of SEQ ID NO: 1).

4. The immunogen of claim 1, wherein each fusion protein comprises an amino acid sequence at least 90% identical to any one of residues 21-463 of SEQ ID NO: 34, residues 21-895 of SEQ ID NO: 35, residues 21-1327 of SEQ ID NO: 36, or residues 23-912 of SEQ ID NO: 37.

5. The immunogen of claim 1, wherein each fusion protein comprises or consists of an amino acid sequence set forth as residues 21-463 of SEQ ID NO: 34, residues 21-895 of SEQ ID NO: 35, residues 21-1327 of SEQ ID NO: 36, or residues 23-912 of SEQ ID NO: 37.

6. The immunogen of claim 1, wherein each fusion protein consists of an amino acid sequence set forth as residues 21-463 of SEQ ID NO: 34, residues 21-895 of SEQ ID NO: 35, residues 21-1327 of SEQ ID NO: 36, or residues 23-912 of SEQ ID NO: 37.

7. The immunogen of claim 1, conjugated to a heterologous carrier.

8. A nucleic acid molecule encoding the immunogen of claim 1.

9. The nucleic acid molecule of claim 8, operably linked to a promoter.

10. A vector comprising the nucleic acid molecule of claim 8.

11. The vector of claim 10, wherein the vector is an RNA vector.

12. A method of producing an immunogen, comprising:
expressing the nucleic acid molecule of claim 8 in a host cell; and
purifying the immunogen.

13. The immunogen produced by the method of claim 12.

14. An immunogenic composition, comprising the immunogen or claim 1, or a nucleic acid molecule encoding the immunogen, and a pharmaceutically acceptable carrier.

15. A method of eliciting an immune response to NiV G protein in a subject, comprising administering to the subject an effective amount of the immunogenic composition of claim 14 to elicit the immune response.

16. The method of claim 15, wherein the immune response treats or inhibits NiV infection in the subject.

17. An immunogen, comprising:
a protein nanoparticle comprising one or more NiV G ectodomains extending radially outward from a globular surface of the nanoparticle, the protein nanoparticle comprising self-assembling protein nanoparticle subunits, wherein each subunit is fused N-terminally to one or more NiV G ectodomains.

18. The immunogen of claim 17, wherein the protein nanoparticle is a ferritin nanoparticle and the nanoparticle subunits are ferritin subunits.

19. The immunogen of claim 17, wherein the fusion proteins in the nanoparticle comprise or consist of an amino acid sequence set forth as any one of residues 57-652 of SEQ ID NO: 38, residues 57-661 of SEQ ID NO: 39, residues 57-671 of SEQ ID NO: 40, residues 57-681 of SEQ ID NO: 41, residues 57-647 of SEQ ID NO: 42, or a sequence at least 90% identical to any one of residues 57-652 of SEQ ID NO: 38, residues 57-661 of SEQ ID NO: 39, residues 57-671 of SEQ ID NO: 40, residues 57-681 of SEQ ID NO: 41, residues 57-647 of SEQ ID NO: 42.


SEQUENCE LISTING (70 sequences)

SEQ ID NO: 1 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSDMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDDSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 2 - protein, 528 aa, synthetic construct
  REGION 1..528: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNSGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 3 - protein, 530 aa, synthetic construct
  REGION 1..530: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNGSGAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 4 - protein, 534 aa, synthetic construct
  REGION 1..534: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTGSGIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 5 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDCVGDVRLAGVCMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 6 - protein, 529 aa, synthetic construct
  REGION 1..529: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNGGSGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 7 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDLVGDVRLAGVCMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVCISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 8 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSDMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVFTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDDSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 9 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 10 - protein, 526 aa, synthetic construct
  REGION 1..526: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 11 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDWINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 12 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITCGVALYEAMKNADNINKLKSSIESTNEAVVKLQETAEKTVYVLTALQDYINTNLVPTIDKISCKQTELSLDLALSKYLSDLLFVFGPNLQDPCSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 13 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSNMSGCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 14 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIGKIGCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 15 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSDMSQCTGSVMENYKTRLNGILTPIKGALEIYKNNTHDLVGDVRLAGVIMAGVAIGIATAAQITAGVALYEAMKNADNINKLKSSIESTNEAVVKLCETAEKCVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDDSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 16 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTFQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 17 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDLVGDVRLAGVCMAGVAIGIATAAQITAGVALYEAMKNADNINKLKSSIESTNEAVVKLQETAEKTVYVFTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVCISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 18 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDCVGDVRLAGVCMAGVAIGIATAAQITAGVALYEAMKNADNINKLKSSIESTNEAVVKLQETAEKTVYVFTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 19 - protein, 534 aa, synthetic construct
  REGION 1..534: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTGSGIMAGVAIGIATAAQITAGVALYEAMKNADNINKLKSSIESTNEAVVKLQETAEKTVYVFTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 20 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDLVGDVRLAGVCMAGVAIGIATAAQITAGVALYEAMKNADNINKLKSSIESTNEAVVKLQETAEKTVYVLTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVCISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 21 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDCVGDVRLAGVCMAGVAIGIATAAQITAGVALYEAMKNADNINKLKSSIESTNEAVVKLQETAEKTVYVLTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 22 - protein, 534 aa, synthetic construct
  REGION 1..534: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTGSGIMAGVAIGIATAAQITAGVALYEAMKNADNINKLKSSIESTNEAVVKLQETAEKTVYVLTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 23 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDLVGDVRLAGVCMAGVAIGIATAAQITAGVALYEAMKNADNINKLKSSIESTNEAVVKLQETAEKTVYVFTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVCISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 24 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDCVGDVRLAGVCMAGVAIGIATAAQITAGVALYEAMKNADNINKLKSSIESTNEAVVKLQETAEKTVYVFTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 25 - protein, 534 aa, synthetic construct
  REGION 1..534: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTGSGIMAGVAIGIATAAQITAGVALYEAMKNADNINKLKSSIESTNEAVVKLQETAEKTVYVFTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 26 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVFTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 27 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSNMSGCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVFTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 28 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSNMSGCTGSVMENYKTRLNGILTPIKGALEIYKNNTHDCVGDVRLAGVCMAGVAIGIATAAQITAGVALYEAMKNADNINKLKSSIESTNEAVVKLQETAEKTVYVFTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 29 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSNMSGCTGSVMENYKTRLNGILTPIKGALEIYKNNTHDCVGDVRLAGVCMAGVAIGIATAAQITAGVALYEAMKNADNINKLKSSIESTNEAVVKLQETAEKTVYVFTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 30 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSNMSGCTGSVMENYKTRLNGILTPIKGALEIYKNNTHDCVGDVRLAGVCMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 31 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDCVGDVRLAGVCMAGVAIGIATAAQITAGVALYEAMKNADNINKLKSSIESTNEAVVKLCETAEKCVYVFTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 32 - protein, 543 aa, synthetic construct
  REGION 1..543: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDCVGDVRLAGVCMAGVAIGIATAAQITCGVALYEAMKNADNINKLKSSIESTNEAVVKLQETAEKTVYVFTALQDYINTNLVPTIDKIPCKQTELSLDLALSKYLSDLLFVFGPNLQDPCSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 33 - DNA, 1662 bp, synthetic construct
  misc_feature 1..1662: note = recombinant nipah virus F sequence
  source 1..1662: mol_type = other DNA
tctagagccaccatggtggtcatcctggacaagagatgctactgtaacctgctgatcctgatcctgatgatcagcgagtgctccgtgggcatcctgcactacgagaagctgtccaagatcggcctggtgaagggcgtgaccaggaagtataagatcaagtctaatcccctgacaaaggatatcgtgatcaagatgatccctaacgtgtctaatatgagccagtgtaccggctccgtgatggagaactacaagaccagactgaatggcatcctgacacccatcaagggcgccctggagatctataagaacaatacacacgactgcgtgggcgatgtgaggctggcaggcgtgtgcatggcaggagtggcaatcggaatcgcaaccgcagcacagatcacagcaggagtggccctgtatgaggccatgaagaacgccgacaacatcaataagctgaagagctccatcgagagcaccaatgaggccgtggtgaagctgcaggagaccgccgagaagacagtgtacgtgttcacagccctgcaggactatatcaacaccaatctggtgcctacaatcgataagatcccttgcaagcagaccgagctgagcctggacctggccctgagcaagtacctgtccgatctgctgttcgtgtttggcccaaacctgcaggaccccgtgagcaattccatgacaatccaggccatctcccaggccttcggcggcaactacgagaccctgctgcgcacactgggctatgccaccgaggactttgacgatctgctggagtctgatagcatcacaggccagatcatctatgtggacctgtctagctactatatcatcgtgcgggtgtacttcccaatcctgaccgagatccagcaggcctatatccaggagctgctgcccgtgtccttcaacaatgataactctgagtggatcagcatcgtgcctaacttcatcctggtgcggaacaccctgatctctaatatcgagatcggcttttgcctgatcacaaagcgcagcgtgatctgtaatcaggactacgccacccctatgacaaacaatatgcgggagtgcctgaccggcagcacagagaagtgtcctcgggagctggtggtgtcctctcacgtgccaagattcgccctgtccaacggcgtgctgtttgccaattgcatctctgtgacctgccagtgtcagaccacaggcagggccatctcccagtctggcgagcagaccctgctgatgatcgataacaccacatgtccaacagccgtgctgggcaatgtgatcatcagcctgggcaagtacctgggcagcgtgaactataattccgagggaatcgcaatcggaccacccgtgttcaccgacaaggtggatatcagctcccagatctctagcatgaaccagtccctgcagcagtctaaggactacatcaaggaggcccagcgcctgctggataccgtgaatccatccctgatctctatgctgagcatgatcatcctgtatgtgctgtccatcgcctctctgtgcatcggcctgatcaccttcatcagctttatcatcgtggagaagaagaggaacacatactcccgcctggaggacaggagagtgcggcccacctcctctggcgatctgtactatatcggcacatgatgaggatcc

SEQ ID NO: 34 - protein, 507 aa, synthetic construct
  REGION 1..507: note = Recombinant Nipah virus G sequence
MYSMQLASCVTLTLVLLVNSQGSGYIPEAPRDGQAYVRKDGEWVLLSTFLGSGGGGGGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGLVPRGSHHHHHHSAWSHPQFEK

SEQ ID NO: 35 - protein, 939 aa, synthetic construct
  REGION 1..939: note = Recombinant Nipah virus G sequence
MYSMQLASCVTLTLVLLVNSQGSGYIPEAPRDGQAYVRKDGEWVLLSTFLGSGGGGGGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGGGGGGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGLVPRGSHHHHHHSAWSHPQFEK

SEQ ID NO: 36 - protein, 1371 aa, synthetic construct
  REGION 1..1371: note = Recombinant Nipah virus G sequence
MYSMQLASCVTLTLVLLVNSQGSGYIPEAPRDGQAYVRKDGEWVLLSTFLGSGGGGGGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGGGGGGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGGGGGGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGLVPRGSHHHHHHSAWSHPQFEK

SEQ ID NO: 37 - protein, 936 aa, synthetic construct
  REGION 1..936: note = Recombinant Nipah virus G sequence
MYSMQLASCVTLTLVLLVNSQEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGGSGYIPEAPRDGQAYVRKDGEWVLLSTFLGSGGGGGGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGLVPRGSHHHHHHSAWSHPQFEK

SEQ ID NO: 38 - protein, 652 aa, synthetic construct
  REGION 1..652: note = Recombinant Nipah virus G sequence
MYSMQLASCVTLTLVLLVNSQHHHHHHGSAWSHPQFEKGGLVPRGSGNSQRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGGSGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 39 - protein, 661 aa, synthetic construct
  REGION 1..661: note = Recombinant Nipah virus G sequence
MYSMQLASCVTLTLVLLVNSQHHHHHHGSAWSHPQFEKGGLVPRGSGNSQRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGGSGGGGSGGSGGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 40 - protein, 671 aa, synthetic construct
  REGION 1..671: note = Recombinant Nipah virus G sequence
MYSMQLASCVTLTLVLLVNSQHHHHHHGSAWSHPQFEKGGLVPRGSGNSQRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGGSGGGGSGGSGGGGSGGGGSGGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 41 - protein, 681 aa, synthetic construct
  REGION 1..681: note = Recombinant Nipah virus G sequence
MYSMQLASCVTLTLVLLVNSQHHHHHHGSAWSHPQFEKGGLVPRGSGNSQRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGGSGGGGSGGSGGGGSGGGGSGGGGSGGGGSGGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 42 - protein, 647 aa, synthetic construct
  REGION 1..647: note = Recombinant Nipah virus G sequence
MYSMQLASCVTLTLVLLVNSQHHHHHHGSAWSHPQFEKGGLVPRGSGNSQRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGGSGGGSGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLADLSLELRKPITFGVITADTLEQAIERAGTKHGNKGWEAALSAIEMANLFKSLR

SEQ ID NO: 43 - protein, 1005 aa, synthetic construct
  REGION 1..1005: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDCVGDVRLAGVCMAGVAIGIATAAQITAGVALYEAMKNADNINKLKSSIESTNEAVVKLQETAEKTVYVFTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGSGYIPEAPRDGQAYVRKDGEWVLLSTFLGSGGGGGGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGLVPRGSHHHHHHSAWSHPQFEK

SEQ ID NO: 44 - protein, 1007 aa, synthetic construct
  REGION 1..1007: note = Recombinant Nipah virus F sequence
MYSMQLASCVTLTLVLLVNSQRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGGQGILHYEKLSKIGLVKGVTRKYKIKSNPLTKDIVIKMIPNVSNMSQCTGSVMENYKTRLNGILTPIKGALEIYKNNTHDCVGDVRLAGVCMAGVAIGIATAAQITAGVALYEAMKNADNINKLKSSIESTNEAVVKLQETAEKTVYVFTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGSGYIPEAPRDGQAYVRKDGEWVLLSTFLGSLVPRGSHHHHHHSAWSHPQFEK

SEQ ID NO: 45 - protein, 991 aa, synthetic construct
  REGION 1..991: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNGGSGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGSGYIPEAPRDGQAYVRKDGEWVLLSTFLGSGGGGGGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGLVPRGSHHHHHHSAWSHPQFEK

SEQ ID NO: 46 - protein, 1423 aa, synthetic construct
  REGION 1..1423: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNGGSGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGSGYIPEAPRDGQAYVRKDGEWVLLSTFLGSGGGGGGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGGGGGGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGLVPRGSHHHHHHSAWSHPQFEK

SEQ ID NO: 47 - protein, 1437 aa, synthetic construct
  REGION 1..1437: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDCVGDVRLAGVCMAGVAIGIATAAQITAGVALYEAMKNADNINKLKSSIESTNEAVVKLQETAEKTVYVFTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGSGYIPEAPRDGQAYVRKDGEWVLLSTFLGSGGGGGGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGGGGGGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGLVPRGSHHHHHHSAWSHPQFEK

SEQ ID NO: 48 - protein, 1855 aa, synthetic construct
  REGION 1..1855: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNGGSGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGSGYIPEAPRDGQAYVRKDGEWVLLSTFLGSGGGGGGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGGGGGGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGGGGGGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGLVPRGSHHHHHHSAWSHPQFEK

SEQ ID NO: 49 - protein, 1869 aa, synthetic construct
  REGION 1..1869: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDCVGDVRLAGVCMAGVAIGIATAAQITAGVALYEAMKNADNINKLKSSIESTNEAVVKLQETAEKTVYVFTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGSGYIPEAPRDGQAYVRKDGEWVLLSTFLGSGGGGGGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGGGGGGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGGGGGGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGLVPRGSHHHHHHSAWSHPQFEK

SEQ ID NO: 50 - protein, 966 aa, synthetic construct
  REGION 1..966: note = Recombinant Nipah virus F sequence
MYSMQLASCVTLTLVLLVNSQRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGGGGQGILHYEKLSKIGLVKGVTRKYKIKSNPLTKDIVIKMIPNVSNMSQCTGSVMENYKTRLNGILTPIKGALEIYKNGGSGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGGLVPRGSHHHHHHSAWSHPQFEK

SEQ ID NO: 51 - protein, 1013 aa, synthetic construct
  REGION 1..1013: note = Recombinant Nipah virus F sequence
MYSMQLASCVTLTLVLLVNSQRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGGGSGGGGQGILHYEKLSKIGLVKGVTRKYKIKSNPLTKDIVIKMIPNVSNMSQCTGSVMENYKTRLNGILTPIKGALEIYKNNTHDCVGDVRLAGVCMAGVAIGIATAAQITAGVALYEAMKNADNINKLKSSIESTNEAVVKLQETAEKTVYVFTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGSGYIPEAPRDGQAYVRKDGEWVLLSTFLGSLVPRGSHHHHHHSAWSHPQFEK

SEQ ID NO: 52 - protein, 546 aa, Nipah henipavirus
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 53 - protein, 602 aa, Nipah henipavirus
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 54 - protein, 602 aa, Nipah henipavirus
MPTESKKVRFENTASDKGKNPSKVIKSYYGTMDIKKINEGLLDSKILSAFNTVIALLGSIVIIVMNIMIIQNYTRSTDNQAMIKDALQSIQQQIKGLADKIGTEIGPKVSLIDTSSTITIPANIGLLGSKISQSTASINENVNEKCKFTLPPLKIHECNISCPNPLPFREYKPQTEGVSNLVGLPNNICLQKTSNQILKPKLISYTLPVVGQSGTCITDPLLAMDEGYFAYSHLEKIGSCSRGVSKQRIIGVGEVLDRGDEVPSLFMTNVWTPSNPNTVYHCSAVYNNEFYYVLCAVSVVGDPILNSTYWSGSLMMTRLAVKPKNNGESYNQHQFALRNIEKGKYDKVMPYGPSGIKQGDTLYFPAVGFLVRTEFKYNDSNCPIAECQYSKPENCRLSMGIRPNSHYILRSGLLKYNLSDEENSKIVFIEISDQRLSIGSPSKIYDSLGQPVFYQASFSWDTMIKFGDVQTVNPLVVNWRDNTVISRPGQSQCPRFNKCPEVCWEGVYNDAFLIDRINWISAGVFLDSNQTAENPVFTVFKDNEVLYRAQLASEDTNAQKTITNCFLLKNKIWCISLVEIYDTGDNVIRPKLFAVKIPEQCT

SEQ ID NO: 55 - protein, 163 aa, synthetic construct
  REGION 1..163: note = Recombinant ferritin sequence
DIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 56 - protein, 154 aa, synthetic construct
  REGION 1..154: note = Recombinant lumazine synthase sequence
MQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLADLSLELRKPITFGVITADTLEQAIERAGTKHGNKGWEAALSAIEMANLFKSLR

SEQ ID NO: 57 - protein, 265 aa, synthetic construct [sequence omitted]

SEQ ID NO: 58 - protein, 265 aa, synthetic construct [sequence omitted]

SEQ ID NO: 59 - protein, 976 aa, synthetic construct
  REGION 1..976: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDCVGDVRLAGVCMAGVAIGIATAAQITAGVALYEAMKNADNINKLKSSIESTNEAVVKLQETAEKTVYVFTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGSGGGGGGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGLVPRGSHHHHHHSAWSHPQFEK

SEQ ID NO: 60 - protein, 970 aa, synthetic construct
  REGION 1..970: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDCVGDVRLAGVCMAGVAIGIATAAQITAGVALYEAMKNADNINKLKSSIESTNEAVVKLQETAEKTVYVFTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLGSGYIPEAPRDGQAYVRKDGEWVLLSTFLGSGGGGGGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCTGGLVPRGSHHHHHHSAWSHPQFEK

SEQ ID NO: 61 - DNA, 3038 bp, synthetic construct
  misc_feature 1..3038: note = recombinant Nipah virus F sequence
  source 1..3038: mol_type = other DNA
tctagagccaccatgtactctatgcagctggccagctgcgtgaccctgacactggtgctgctggtgaactctcagggcatcctgcactacgagaagctgagcaagatcggcctggtgaagggcgtgaccagaaagtataagatcaagtccaacccactgacaaaggacatcgtgatcaagatgatccccaacgtgagcaatatgtctcagtgtaccggctctgtgatggagaactacaagacccgcctgaatggcatcctgacaccaatcaagggcgccctggagatctataagaacaatacacacgactgcgtgggcgatgtgcggctggcaggcgtgtgcatggcaggagtggcaatcggaatcgcaaccgcagcacagatcacagcaggagtggccctgtatgaggccatgaagaacgccgacaacatcaataagctgaagagctccatcgagagcaccaatgaggccgtggtgaagctgcaggagacagccgagaagacagtgtacgtgttcacagccctgcaggactatatcaacaccaatctggtgcccacaatcgataagatcccctgcaagcagaccgagctgtccctggacctggccctgtctaagtacctgagcgatctgctgttcgtgtttggcccaaacctgcaggaccccgtgtccaattctatgacaatccaggccatctcccaggccttcggcggcaattacgagacactgctgagaacactgggctatgccaccgaggactttgacgatctgctggagagcgattccatcacaggccagatcatctatgtggatctgtctagctactatatcatcgtgagggtgtacttccctatcctgaccgagatccagcaggcctatatccaggagctgctgccagtgagcttcaacaatgacaattccgagtggatctctatcgtgcccaactttatcctggtgcggaacaccctgatcagcaatatcgagatcggcttttgcctgatcacaaagagatccgtgatctgtaatcaggactacgccacccccatgacaaacaatatgagggagtgcctgaccggctccacagagaagtgtccccgggagctggtggtgtcctctcacgtgcctagattcgccctgtccaacggcgtgctgtttgccaattgcatctctgtgacctgccagtgtcagaccacaggaagggcaatctctcagagcggagagcagaccctgctgatgatcgataacaccacatgtcctacagccgtgctgggcaatgtgatcatcagcctgggcaagtacctgggctccgtgaactataattctgagggaatcgcaatcggaccacccgtgttcaccgacaaggtggatatcagctcccagatctctagcatgaaccagagcctgcagcagtccaaggactacatcaaggaggcccagcggctgctggataccgtgaatccttctctgaagctgatgaagcagatcgaggataagatcgaggagatcctgagcaagatctatcacatcgagaacgagatcgccaggatcaagaagctgatcggagaggcacctggatctggttacatcccagaggctccgcgggatggacaggcctacgtgagaaaggacggcgagtgggtgctgctgagcaccttcctgggaagcggtggaggaggcggaggcgtgagcaatctggtgggcctgcccaacaatatctgtctgcagaagacctccaaccagatcctgaagcccaagctgatctcctatacactgcctgtggtgggccagtctggcacctgcatcacagaccctctgctggccatggatgagggctacttcgcctattctcacctggagaggatcggctcctgttctcgcggcgtgagcaagcagcggatcatcggagtgggagaggtgctggacaggggcgatgaggtgccttccctgttcatgaccaacgtgtggacaccacccaatccaaacaccgtgtaccactgctctgccgtgtataacaatgagttttactacgtgctgtgcgccgtgagcaccgtgggcgatcctatcctgaactccacatactggagcggctccctgatgatgaccagactggccgtgaagccaaagtccaatggcggcggctataaccagcaccagctggccctgagatctatcgagaagggcaggtacgataaagtgatgccttatggcccatctggcatcaagcagggcgacacactgtacttccccgccgtgggctttctggtgaggaccgagttcaagtacaatgactccaactgccctatcacaaagtgtcagtattctaagccagagaattgccgcctgagcatgggcatccggcccaactctcactacatcctgagaagcggcctgctgaagtataatctgagcgacggcgagaaccctaaggtggtgtttatcgagatctccgatcagaggctgtctatcggctctcccagcaagatctacgactccctgggccagcccgtgttctaccaggcctccttttcttgggacacaatgatcaagttcggcgatgtgctgaccgtgaatccactggtggtgaactggagaaacaataccgtgatcagcaggccaggacagtcccagtgtcctcgctttaacacatgcccagagatctgttgggagggcgtgtacaatgacgccttcctgatcgatcggatcaactggatctccgccggcgtgtttctggactctaatcagaccgccgagaaccccgtgttcacagtgtttaaggataatgagatcctgtacagggcacagctggcaagcgaggacaccaacgcccagaagaccatcacaaattgcttcctgctgaagaacaagatctggtgtatctccctggtggagatctatgacaccggcgataacgtgatccggccaaagctgtttgccgtgaagatccccgagcagtgcacaggcggcctggtgcctagaggctctcaccaccaccatcaccacagcgcctggtcccacccccagttcgagaagtgataggatcc

SEQ ID NO: 62 - DNA, 3044 bp, synthetic construct
  misc_feature 1..3044: note = recombinant Nipah virus F sequence
  source 1..3044: mol_type = other DNA
tctagaccaccatgtactcaatgcagctggcctcttgcgtcacactgacactggtcctgctggtcaactcacagagaccacagaccgagggcgtgagcaatctggtgggcctgcccaacaatatctgtctgcagaagacctccaaccagatcctgaagcccaagctgatctcctatacactgcctgtggtgggccagtctggcacctgcatcacagaccctctgctggccatggatgagggctacttcgcctattctcacctggagaggatcggctcctgttctcgcggcgtgagcaagcagcggatcatcggagtgggagaggtgctggacaggggcgatgaggtgccttccctgttcatgaccaacgtgtggacaccacccaatccaaacaccgtgtaccactgctctgccgtgtataacaatgagttttactacgtgctgtgcgccgtgagcaccgtgggcgatcctatcctgaactccacatactggagcggctccctgatgatgaccagactggccgtgaagccaaagtccaatggcggcggctataaccagcaccagctggccctgagatctatcgagaagggcaggtacgataaagtgatgccttatggcccatctggcatcaagcagggcgacacactgtacttccccgccgtgggctttctggtgaggaccgagttcaagtacaatgactccaactgccctatcacaaagtgtcagtattctaagccagagaattgccgcctgagcatgggcatccggcccaactctcactacatcctgagaagcggcctgctgaagtataatctgagcgacggcgagaaccctaaggtggtgtttatcgagatctccgatcagaggctgtctatcggctctcccagcaagatctacgactccctgggccagcccgtgttctaccaggcctccttttcttgggacacaatgatcaagttcggcgatgtgctgaccgtgaatccactggtggtgaactggagaaacaataccgtgatcagcaggccaggacagtcccagtgtcctcgctttaacacatgcccagagatctgttgggagggcgtgtacaatgacgccttcctgatcgatcggatcaactggatctccgccggcgtgtttctggactctaatcagaccgccgagaaccccgtgttcacagtgtttaaggataatgagatcctgtacagggcacagctggcaagcgaggacaccaacgcccagaagaccatcacaaattgcttcctgctgaagaacaagatctggtgtatctccctggtggagatctatgacaccggcgataacgtgatccggccaaagctgtttgccgtgaagatccccgagcagtgcacaggcggcggtcagggcatcctgcactacgagaagctgagcaagatcggcctggtgaagggcgtgaccagaaagtataagatcaagtccaacccactgacaaaggacatcgtgatcaagatgatccccaacgtgagcaatatgtctcagtgtaccggctctgtgatggagaactacaagacccgcctgaatggcatcctgacaccaatcaagggcgccctggagatctataagaacaatacacacgactgcgtgggcgatgtgcggctggcaggcgtgtgcatggcaggagtggcaatcggaatcgcaaccgcagcacagatcacagcaggagtggccctgtatgaggccatgaagaacgccgacaacatcaataagctgaagagctccatcgagagcaccaatgaggccgtggtgaagctgcaggagacagccgagaagacagtgtacgtgttcacagccctgcaggactatatcaacaccaatctggtgcccacaatcgataagatcccctgcaagcagaccgagctgtccctggacctggccctgtctaagtacctgagcgatctgctgttcgtgtttggcccaaacctgcaggaccccgtgtccaattctatgacaatccaggccatctcccaggccttcggcggcaattacgagacactgctgagaacactgggctatgccaccgaggactttgacgatctgctggagagcgattccatcacaggccagatcatctatgtggatctgtctagctactatatcatcgtgagggtgtacttccctatcctgaccgagatccagcaggcctatatccaggagctgctgccagtgagcttcaacaatgacaattccgagtggatctctatcgtgcccaactttatcctggtgcggaacaccctgatcagcaatatcgagatcggcttttgcctgatcacaaagagatccgtgatctgtaatcaggactacgccacccccatgacaaacaatatgagggagtgcctgaccggctccacagagaagtgtccccgggagctggtggtgtcctctcacgtgcctagattcgccctgtccaacggcgtgctgtttgccaattgcatctctgtgacctgccagtgtcagaccacaggaagggcaatctctcagagcggagagcagaccctgctgatgatcgataacaccacatgtcctacagccgtgctgggcaatgtgatcatcagcctgggcaagtacctgggctccgtgaactataattctgagggaatcgcaatcggaccacccgtgttcaccgacaaggtggatatcagctcccagatctctagcatgaaccagagcctgcagcagtccaaggactacatcaaggaggcccagcggctgctggataccgtgaatccttctctgaagctgatgaagcagatcgaggataagatcgaggagatcctgagcaagatctatcacatcgagaacgagatcgccaggatcaagaagctgatcggagaggcacctggatctggttacatcccagaggctccgcgggatggacaggcctacgtgagaaaggacggcgagtgggtgctgctgagcaccttcctgggaagcctggtgcctaggggctcccaccaccaccaccaccacagcgcctggtcccacccacagtttgagaagtgatgaggatcc

SEQ ID NO: 63 - DNA, 1653 bp, synthetic construct
  misc_feature 1..1653: note = recombinant nipah virus sequence
  source 1..1653: mol_type = other DNA
tctagagccaccatgtactctatgcagctggccagctgcgtgaccctgacactggtgctgctggtgaactctcagggcatcctgcactacgagaagctgagcaagatcggcctggtgaagggcgtgaccagaaagtataagatcaagtccaacccactgacaaaggacatcgtgatcaagatgatccccaacgtgagcaatatgtctcagtgtaccggctctgtgatggagaactacaagacccgcctgaatggcatcctgacaccaatcaagggcgccctggagatctataagaacaatacacacgactgcgtgggcgatgtgcggctggcaggcgtgtgcatggcaggagtggcaatcggaatcgcaaccgcagcacagatcacagcaggagtggccctgtatgaggccatgaagaacgccgacaacatcaataagctgaagagctccatcgagagcaccaatgaggccgtggtgaagctgcaggagacagccgagaagacagtgtacgtgttcacagccctgcaggactatatcaacaccaatctggtgcccacaatcgataagatcccctgcaagcagaccgagctgtccctggacctggccctgtctaagtacctgagcgatctgctgttcgtgtttggcccaaacctgcaggaccccgtgtccaattctatgacaatccaggccatctcccaggccttcggcggcaattacgagacactgctgagaacactgggctatgccaccgaggactttgacgatctgctggagagcgattccatcacaggccagatcatctatgtggatctgtctagctactatatcatcgtgagggtgtacttccctatcctgaccgagatccagcaggcctatatccaggagctgctgccagtgagcttcaacaatgacgattccgagtggatctctatcgtgcccaactttatcctggtgcggaacaccctgatcagcaatatcgagatcggcttttgcctgatcacaaagagatccgtgatctgtaatcaggactacgccacccccatgacaaacaatatgagggagtgcctgaccggctccacagagaagtgtccccgggagctggtggtgtcctctcacgtgcctagattcgccctgtccaacggcgtgctgtttgccaattgcatctctgtgacctgccagtgtcagaccacaggaagggcaatctctcagagcggagagcagaccctgctgatgatcgataacaccacatgtcctacagccgtgctgggcaatgtgatcatcagcctgggcaagtacctgggctccgtgaactataattctgagggaatcgcaatcggaccacccgtgttcaccgacaaggtggatatcagctcccagatctctagcatgaaccagagcctgcagcagtccaaggactacatcaaggaggcccagcggctgctggataccgtgaatccttctctgaagctgatgaagcagatcgaggataagatcgaggagatcctgagcaagatctatcacatcgagaacgagatcgccaggatcaagaagctgatcggagaggcacctggaggcctggtgccaaggggctcccaccaccaccaccaccacagcgcctggtcccacccacagtttgagaagtgatgaggatcc

SEQ ID NO: 64 - DNA, 1979 bp, synthetic construct
  misc_feature 1..1979: note = recombinant nipah virus G sequence
  source 1..1979: mol_type = other DNA
tctagaccaccatgtactcaatgcagctggcctcttgcgtcacactgacactggtcctgctggtcaactcacagcaccaccaccatcatcacggaagcgcctggtcccaccctcagttcgagaagggaggactggtgcccagaggatccggcaacagccagcgccctcagaccgagggcgtgagcaatctggtgggcctgcccaacaatatctgtctgcagaagacctccaaccagatcctgaagcccaagctgatctcctatacactgcctgtggtgggccagtctggcacctgcatcacagaccctctgctggccatggatgagggctacttcgcctattctcacctggagaggatcggctcctgttctcgcggcgtgagcaagcagcggatcatcggagtgggagaggtgctggacaggggcgatgaggtgccttccctgttcatgaccaacgtgtggacaccacccaatccaaacaccgtgtaccactgctctgccgtgtataacaatgagttttactacgtgctgtgcgccgtgagcaccgtgggcgatcctatcctgaactccacatactggagcggctccctgatgatgaccagactggccgtgaagccaaagtccaatggcggcggctataaccagcaccagctggccctgagatctatcgagaagggcaggtacgataaagtgatgccttatggcccatctggcatcaagcagggcgacacactgtacttccccgccgtgggctttctggtgaggaccgagttcaagtacaatgactccaactgccctatcacaaagtgtcagtattctaagccagagaattgccgcctgagcatgggcatccggcccaactctcactacatcctgagaagcggcctgctgaagtataatctgagcgacggcgagaaccctaaggtggtgtttatcgagatctccgatcagaggctgtctatcggctctcccagcaagatctacgactccctgggccagcccgtgttctaccaggcctccttttcttgggacacaatgatcaagttcggcgatgtgctgaccgtgaatccactggtggtgaactggagaaacaataccgtgatcagcaggccaggacagtcccagtgtcctcgctttaacacatgcccagagatctgttgggagggcgtgtacaatgacgccttcctgatcgatcggatcaactggatctccgccggcgtgtttctggactctaatcagaccgccgagaaccccgtgttcacagtgtttaaggataatgagatcctgtacagggcacagctggcaagcgaggacaccaacgcccagaagaccatcacaaattgcttcctgctgaagaacaagatctggtgtatctccctggtggagatctatgacaccggcgataacgtgatccggccaaagctgtttgccgtgaagatccccgagcagtgcacaggcggcggcagcggcggggatatcattaagctgctgaacgaacaggtgaacaaggagatgcagtcaagcaacctgtacatgtctatgtcctcttggtgctatacacatagtctggacggagctggcctgttcctgtttgatcacgcagccgaggaatacgaacatgcaaagaaactgatcattttcctgaatgagaacaatgtgccagtccagctgacaagtatctcagcccccgaacacaagttcgaggggctgactcagatctttcagaaagcttacgaacacgagcagcatattagcgaatccatcaacaatattgtggaccacgctatcaagtccaaagatcatgcaaccttcaactttctgcagtggtacgtggccgagcagcacgaggaagaggtcctgtttaaggacatcctggataaaatcgaactgattggcaacgagaatcatgggctgtacctggccgatcagtatgtgaagggcattgctaagtcacggaaaagcggaagctgatgaccgcgg

SEQ ID NO: 65 - DNA, 2951 bp, synthetic construct
  misc_feature 1..2951: note = recombinant nipah virus F sequence
  source 1..2951: mol_type = other DNA
tctagagccaccatgtactctatgcagctggccagctgcgtgaccctgacactggtgctgctggtgaactctcagggcatcctgcactacgagaagctgagcaagatcggcctggtgaagggcgtgaccagaaagtataagatcaagtccaacccactgacaaaggacatcgtgatcaagatgatccccaacgtgagcaatatgtctcagtgtaccggctctgtgatggagaactacaagacccgcctgaatggcatcctgacaccaatcaagggcgccctggagatctataagaacaatacacacgactgcgtgggcgatgtgcggctggcaggcgtgtgcatggcaggagtggcaatcggaatcgcaaccgcagcacagatcacagcaggagtggccctgtatgaggccatgaagaacgccgacaacatcaataagctgaagagctccatcgagagcaccaatgaggccgtggtgaagctgcaggagacagccgagaagacagtgtacgtgttcacagccctgcaggactatatcaacaccaatctggtgcccacaatcgataagatcccctgcaagcagaccgagctgtccctggacctggccctgtctaagtacctgagcgatctgctgttcgtgtttggcccaaacctgcaggaccccgtgtccaattctatgacaatccaggccatctcccaggccttcggcggcaattacgagacactgctgagaacactgggctatgccaccgaggactttgacgatctgctggagagcgattccatcacaggccagatcatctatgtggatctgtctagctactatatcatcgtgagggtgtacttccctatcctgaccgagatccagcaggcctatatccaggagctgctgccagtgagcttcaacaatgacaattccgagtggatctctatcgtgcccaactttatcctggtgcggaacaccctgatcagcaatatcgagatcggcttttgcctgatcacaaagagatccgtgatctgtaatcaggactacgccacccccatgacaaacaatatgagggagtgcctgaccggctccacagagaagtgtccccgggagctggtggtgtcctctcacgtgcctagattcgccctgtccaacggcgtgctgtttgccaattgcatctctgtgacctgccagtgtcagaccacaggaagggcaatctctcagagcggagagcagaccctgctgatgatcgataacaccacatgtcctacagccgtgctgggcaatgtgatcatcagcctgggcaagtacctgggctccgtgaactataattctgagggaatcgcaatcggaccacccgtgttcaccgacaaggtggatatcagctcccagatctctagcatgaaccagagcctgcagcagtccaaggactacatcaaggaggcccagcggctgctggataccgtgaatccttctctgaagctgatgaagcagatcgaggataagatcgaggagatcctgagcaagatctatcacatcgagaacgagatcgccaggatcaagaagctgatcggagaggcacctggatctggtggaggaggcggaggcgtgagcaatctggtgggcctgcccaacaatatctgtctgcagaagacctccaaccagatcctgaagcccaagctgatctcctatacactgcctgtggtgggccagtctggcacctgcatcacagaccctctgctggccatggatgagggctacttcgcctattctcacctggagaggatcggctcctgttctcgcggcgtgagcaagcagcggatcatcggagtgggagaggtgctggacaggggcgatgaggtgccttccctgttcatgaccaacgtgtggacaccacccaatccaaacaccgtgtaccactgctctgccgtgtataacaatgagttttactacgtgctgtgcgccgtgagcaccgtgggcgatcctatcctgaactccacatactggagcggctccctgatgatgaccagactggccgtgaagccaaagtccaatggcggcggctataaccagcaccagctggccctgagatctatcgagaagggcaggtacgataaagtgatgccttatggcccatctggcatcaagcagggcgacacactgtacttccccgccgtgggctttctggtgaggaccgagttcaagtacaatgactccaactgccctatcacaaagtgtcagtattctaagccagagaattgccgcctgagcatgggcatccggcccaactctcactacatcctgagaagcggcctgctgaagtataatctgagcgacggcgagaaccctaaggtggtgtttatcgagatctccgatcagaggctgtctatcggctctcccagcaagatctacgactccctgggccagcccgtgttctaccaggcctccttttcttgggacacaatgatcaagttcggcgatgtgctgaccgtgaatccactggtggtgaactggagaaacaataccgtgatcagcaggccaggacagtcccagtgtcctcgctttaacacatgcccagagatctgttgggagggcgtgtacaatgacgccttcctgatcgatcggatcaactggatctccgccggcgtgtttctggactctaatcagaccgccgagaaccccgtgttcacagtgtttaaggataatgagatcctgtacagggcacagctggcaagcgaggacaccaacgcccagaagaccatcacaaattgcttcctgctgaagaacaagatctggtgtatctccctggtggagatctatgacaccggcgataacgtgatccggccaaagctgtttgccgtgaagatccccgagcagtgcacaggcggcctggtgcctagaggctctcaccaccaccatcaccacagcgcctggtcccacccccagttcgagaagtgataggatcc

SEQ ID NO: 66 - DNA, 2933 bp, synthetic construct
  misc_feature 1..2933: note = recombinant nipah virus F sequence
  source 1..2933: mol_type = other DNA
tctagagccaccatgtactctatgcagctggccagctgcgtgaccctgacactggtgctgctggtgaactctcagggcatcctgcactacgagaagctgagcaagatcggcctggtgaagggcgtgaccagaaagtataagatcaagtccaacccactgacaaaggacatcgtgatcaagatgatccccaacgtgagcaatatgtctcagtgtaccggctctgtgatggagaactacaagacccgcctgaatggcatcctgacaccaatcaagggcgccctggagatctataagaacaatacacacgactgcgtgggcgatgtgcggctggcaggcgtgtgcatggcaggagtggcaatcggaatcgcaaccgcagcacagatcacagcaggagtggccctgtatgaggccatgaagaacgccgacaacatcaataagctgaagagctccatcgagagcaccaatgaggccgtggtgaagctgcaggagacagccgagaagacagtgtacgtgttcacagccctgcaggactatatcaacaccaatctggtgcccacaatcgataagatcccctgcaagcagaccgagctgtccctggacctggccctgtctaagtacctgagcgatctgctgttcgtgtttggcccaaacctgcaggaccccgtgtccaattctatgacaatccaggccatctcccaggccttcggcggcaattacgagacactgctgagaacactgggctatgccaccgaggactttgacgatctgctggagagcgattccatcacaggccagatcatctatgtggatctgtctagctactatatcatcgtgagggtgtacttccctatcctgaccgagatccagcaggcctatatccaggagctgctgccagtgagcttcaacaatgacaattccgagtggatctctatcgtgcccaactttatcctggtgcggaacaccctgatcagcaatatcgagatcggcttttgcctgatcacaaagagatccgtgatctgtaatcaggactacgccacccccatgacaaacaatatgagggagtgcctgaccggctccacagagaagtgtccccgggagctggtggtgtcctctcacgtgcctagattcgccctgtccaacggcgtgctgtttgccaattgcatctctgtgacctgccagtgtcagaccacaggaagggcaatctctcagagcggagagcagaccctgctgatgatcgataacaccacatgtcctacagccgtgctgggcaatgtgatcatcagcctgggcaagtacctgggctccgtgaactataattctgagggaatcgcaatcggaccacccgtgttcaccgacaaggtggatatcagctcccagatctctagcatgaaccagagcctgcagcagtccaaggactacatcaaggaggcccagcggctgctggataccgtgaatccttctctgggatctggttacatcccagaggctccgcgggatggacaggcctacgtgagaaaggacggcgagtgggtgctgctgagcaccttcctgggaagcggtggaggaggcggaggcgtgagcaatctggtgggcctgcccaacaatatctgtctgcagaagacctccaaccagatcctgaagcccaagctgatctcctatacactgcctgtggtgggccagtctggcacctgcatcacagaccctctgctggccatggatgagggctacttcgcctattctcacctggagaggatcggctcctgttctcgcggcgtgagcaagcagcggatcatcggagtgggagaggtgctggacaggggcgatgaggtgccttccctgttcatgaccaacgtgtggacaccacccaatccaaacaccgtgtaccactgctctgccgtgtataacaatgagttttactacgtgctgtgcgccgtgagcaccgtgggcgatcctatcctgaactccacatactggagcggctccctgatgatgaccagactggccgtgaagccaaagtccaatggcggcggctataaccagcaccagctggccctgagatctatcgagaagggcaggtacgataaagtgatgccttatggcccatctggcatcaagcagggcgacacactgtacttccccgccgtgggctttctggtgaggaccgagttcaagtacaatgactccaactgccctatcacaaagtgtcagtattctaagccagagaattgccgcctgagcatgggcatccggcccaactctcactacatcctgagaagcggcctgctgaagtataatctgagcgacggcgagaaccctaaggtggtgtttatcgagatctccgatcagaggctgtctatcggctctcccagcaagatctacgactccctgggccagcccgtgttctaccaggcctccttttcttgggacacaatgatcaagttcggcgatgtgctgaccgtgaatccactggtggtgaactggagaaacaataccgtgatcagcaggccaggacagtcccagtgtcctcgctttaacacatgcccagagatctgttgggagggcgtgtacaatgacgccttcctgatcgatcggatcaactggatctccgccggcgtgtttctggactctaatcagaccgccgagaaccccgtgttcacagtgtttaaggataatgagatcctgtacagggcacagctggcaagcgaggacaccaacgcccagaagaccatcacaaattgcttcctgctgaagaacaagatctggtgtatctccctggtggagatctatgacaccggcgataacgtgatccggccaaagctgtttgccgtgaagatccccgagcagtgcacaggcggcctggtgcctagaggctctcaccaccaccatcaccacagcgcctggtcccacccccagttcgagaagtgataggatcc

SEQ ID NO: 67 - DNA, 2831 bp, synthetic construct
  misc_feature 1..2831: note = recombinant nipah virus F sequence
  source 1..2831: mol_type = other DNA
tctagagccaccatgtactctatgcagctggccagctgcgtgaccctgacactggtgctgctggtgaactctcaggagggcgtgagcaatctggtgggcctgcccaacaatatctgtctgcagaagacctccaaccagatcctgaagcccaagctgatctcctatacactgcctgtggtgggccagtctggcacctgcatcacagaccctctgctggccatggatgagggctacttcgcctattctcacctggagaggatcggctcctgttctcgcggcgtgagcaagcagcggatcatcggagtgggagaggtgctggacaggggcgatgaggtgccttccctgttcatgaccaacgtgtggacaccacccaatccaaacaccgtgtaccactgctctgccgtgtataacaatgagttttactacgtgctgtgcgccgtgagcaccgtgggcgatcctatcctgaactccacatactggagcggctccctgatgatgaccagactggccgtgaagccaaagtccaatggcggcggctataaccagcaccagctggccctgagatctatcgagaagggcaggtacgataaagtgatgccttatggcccatctggcatcaagcagggcgacacactgtacttccccgccgtgggctttctggtgaggaccgagttcaagtacaatgactccaactgccctatcacaaagtgtcagtattctaagccagagaattgccgcctgagcatgggcatccggcccaactctcactacatcctgagaagcggcctgctgaagtataatctgagcgacggcgagaaccctaaggtggtgtttatcgagatctccgatcagaggctgtctatcggctctcccagcaagatctacgactccctgggccagcccgtgttctaccaggcctccttttcttgggacacaatgatcaagttcggcgatgtgctgaccgtgaatccactggtggtgaactggagaaacaataccgtgatcagcaggccaggacagtcccagtgtcctcgctttaacacatgcccagagatctgttgggagggcgtgtacaatgacgccttcctgatcgatcggatcaactggatctccgccggcgtgtttctggactctaatcagaccgccgagaaccccgtgttcacagtgtttaaggataatgagatcctgtacagggcacagctggcaagcgaggacaccaacgcccagaagaccatcacaaattgcttcctgctgaagaacaagatctggtgtatctccctggtggagatctatgacaccggcgataacgtgatccggccaaagctgtttgccgtgaagatccccgagcagtgcacaggcggcggatctggttacatcccagaggctccgcgggatggacaggcctacgtgagaaaggacggcgagtgggtgctgctgagcaccttcctgggaagcggtggaggaggcggaggcgtgagcaatctggtgggcctgcccaacaatatctgtctgcagaagacctccaaccagatcctgaagcccaagctgatctcctatacactgcctgtggtgggccagtctggcacctgcatcacagaccctctgctggccatggatgagggctacttcgcctattctcacctggagaggatcggctcctgttctcgcggcgtgagcaagcagcggatcatcggagtgggagaggtgctggacaggggcgatgaggtgccttccctgttcatgaccaacgtgtggacaccacccaatccaaacaccgtgtaccactgctctgccgtgtataacaatgagttttactacgtgctgtgcgccgtgagcaccgtgggcgatcctatcctgaactccacatactggagcggctccctgatgatgaccagactggccgtgaagccaaagtccaatggcggcggctataaccagcaccagctggccctgagatctatcgagaagggcaggtacgataaagtgatgccttatggcccatctggcatcaagcagggcgacacactgtacttccccgccgtgggctttctggtgaggaccgagttcaagtacaatgactccaactgccctatcacaaagtgtcagtattctaagccagagaattgccgcctgagcatgggcatccggcccaactctcactacatcctgagaagcggcctgctgaagtataatctgagcgacggcgagaaccctaaggtggtgtttatcgagatctccgatcagaggctgtctatcggctctcccagcaagatctacgactccctgggccagcccgtgttctaccaggcctccttttcttgggacacaatgatcaagttcggcgatgtgctgaccgtgaatccactggtggtgaactggagaaacaataccgtgatcagcaggccaggacagtcccagtgtcctcgctttaacacatgcccagagatctgttgggagggcgtgtacaatgacgccttcctgatcgatcggatcaactggatctccgccggcgtgtttctggactctaatcagaccgccgagaaccccgtgttcacagtgtttaaggataatgagatcctgtacagggcacagctggcaagcgaggacaccaacgcccagaagaccatcacaaattgcttcctgctgaagaacaagatctggtgtatctccctggtggagatctatgacaccggcgataacgtgatccggccaaagctgtttgccgtgaagatccccgagcagtgcacaggcggcctggtgcctagaggctctcaccaccaccatcaccacagcgcctggtcccacccccagttcgagaagtgataggatcc

SEQ ID NO: 68 - protein, 436 aa, Hendra henipavirus
REYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDITLQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 69 - protein, 1007 aa, synthetic construct
  REGION 1..1007: note = Recombinant Nipah virus F sequence
MYSMQLASCVTLTLVLLVNSQGILHYEKLSKIGLVKGVTRKYKIKSNPLTKDIVIKMIPNVSNMSQCTGSVMENYKTRLNGILTPIKGALEIYKNNTHDCVGDVRLAGVCMAGVAIGIATAAQITAGVALYEAMKNADNINKLKSSIESTNEAVVKLQETAEKTVYVFTALQDYINTNLVPTIDKIPCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLKLMKQIEDKIEEILSKIYHIENEIARIKKLIGEAPGSGYIPEAPRDGQAYVRKDGEWVLLSTFLGSGGGGGGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDITLQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSESGGLVPRGSHHHHHHSAWSHPQFEK

SEQ ID NO: 70 - protein, 1007 aa, synthetic construct; identical to SEQ ID NO: 44
  REGION 1..1007: note = Recombinant Nipah virus F sequence